SEQUENCE LISTING (6 sequences)

SEQ ID NO: 1 - protein, 176 aa, Homo Sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - DNA, 3349 bp, Homo Sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

SEQ ID NO: 3 - protein, 245 aa, Homo Sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKICLTLSPST

SEQ ID NO: 4 - DNA, 907 bp, Homo Sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccaggtaatattctgaatgtgtccattaaaatatgtctaacactgtcccctagcacctagcatgatgtctgcctatcatagtcattcagtgattgttgaataaatgaatgaatgaataaca

SEQ ID NO: 5 - protein, 290 aa, Homo Sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 6 - DNA, 3634 bp, Homo Sapeins
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

(19) United States
(12) Patent Application Publication
(10) Pub. No.: US 2020/0395097 A1
(43) Pub. Date: Dec. 17, 2020

(54) PAN-CANCER MODEL TO PREDICT THE PD-L1 STATUS OF A CANCER CELL SAMPLE USING RNA EXPRESSION DATA AND OTHER PATIENT DATA

(71) Applicant: TEMPUS LABS, INC., Chicago, IL (US)

(72) Inventors: Alan Chang, Chicago, IL (US); Denise Lau, Chicago, IL (US); Aly Azeem Khan, Chicago, IL (US)

(21) Appl. No.: 16/888,357

(22) Filed: May 29, 2020

Related U.S. Application Data
(60) Provisional application No. 62/854,400, filed on May 30, 2019.

(51) Int. Cl. G16B 30/10; G16B 40/30; G16B 5/20

(52) U.S. Cl. CPC G16B 30/10; G16B 5/20; G16B 40/30

(57) ABSTRACT

Provided herein are computer-implemented methods of identifying programmed-death ligand 1 (PD-L1) expression status of a subject's sample comprising a cancer cell. In exemplary embodiments, the method comprises receiving an unlabeled expression data set for the subject's sample; aligning the unlabeled expression data set to labeled expression data according to a trained PD-L1 predictive model, wherein the trained PD-L1 predictive model has been trained with a plurality of labeled expression data sets, each labeled expression data set comprising expression data for a sample of a labeled cancer type and a labeled PD-L1 expression status; wherein aligning the unlabeled gene expression data set to labeled expression data according to the trained PD-L1 predictive model identifies PD-L1 expression status for the subject's sample. Further provided are related methods of preparing a clinical decision support information (CDSI) report and methods of determining treatment for a subject. Additionally provided are CDSI reports and computing devices.

### CROSS-REFERENCE TO RELATED APPLICATIONS

This application claims priority to provisional U.S. Application Ser. No. 62/854,400, filed on May 30, 2019, entitled, A Pan-Cancer Model To Predict The Pd-L1 Status Of A Cancer Cell Sample Using Rna Expression Data And Other Patient Data, the entire disclosure of which is hereby expressly incorporated by reference herein.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 17,129 byte ASCII (Text) file named “53961_Seqlisting.txt”; created on May 30, 2019.

### BACKGROUND

Physicians treating cancer patients may run tests on their patients' biospecimens to predict what treatment is most likely to treat the patient's cancer. One type of test that physicians may order determines whether their patient's cancer cells create or contain certain biomarkers or another treatment-related molecule of interest. In some instances, the biomarker is programmed death ligand 1 (PD-L1), also known as CD274.

The percentage of cancer cells that express PD-L1 protein in a patient can predict whether immunotherapy treatments, especially immune checkpoint blockade treatments, are likely to successfully eliminate or reduce the number of the patient's cancer cells. Examples of checkpoint blockade treatments are antibodies that target PD-L1 or programmed death ligand 1 (PD-1), the receptor for PD-L1, in order to activate the immune system to eliminate cancer cells

Currently, immunohistochemistry (IHC) staining, fluorescence in situ hybridization (FISH), or reverse phase protein array (RPPA) may be used to detect any treatment-related molecule of interest in tumor tissue or another cancer cell sample.

For IHC staining, a thin slice of tumor tissue (approximately 5 microns thick) or a blood smear of cancer cells is affixed to glass microscope slides to create a histology slide, also known as a pathology slide. The slide is submerged in a liquid solution containing antibodies. Each antibody is designed to bind to one copy of the target biomarker molecule on the slide and is coupled with an enzyme that then converts a substrate into a visible dye. This stain allows a trained pathologist or other trained analyst to visually inspect the location of target molecules on the slide.

A portion of the cells on the slide may be normal cells, and another portion of cells are cancer cells. If a cancer cell on the slide displays IHC staining, it is considered positive for expressing the IHC target, such as PD-L1. Generally, an analyst views the slide to estimate the percentage of the total cancer cells that are positive and compares it to a threshold value. If the percentage exceeds that threshold, the cancer cell sample on the slide is designated as positive for that biomarker.

Similarly, FISH and RPPA can be used to visually detect and quantify copies of the PD-L1 protein and/or CD274 RNA in a cancer cell sample. If the results of these assays exceed a selected threshold value, the cancer cell sample can be labeled as PD-L1 positive.

There are several disadvantages of using IHC, FISH, or RPPA to determine the biomarker status of a cancer cell sample.

The process of conducting IHC staining, FISH, and RPPA requires time, trained technicians, equipment and antibodies or other reagents, all of which can be expensive.

Often, an IHC slide analyst does not have enough time to count all of the cancer cells on an IHC-stained slide and inaccurately estimates the percentage of stained cancer cells by eye. Because the estimate is subjective, any two analysts may disagree when determining whether a slide exceeds a PD-L1 threshold. There are similar challenges for FISH and RPPA.

IHC staining, FISH, and RPPA assays may require up to ten slices of tumor tissue from a biopsy or a sample of blood taken from the patient. Collecting cancer cells through biopsies or blood draws subjects the patient to discomfort and inconvenience, so the amount of cancer cells available for testing is limited. Often, the tissue is needed for other tests, including genetic sequence analysis.

Therefore, there is a need for systems and methods that predict the PD-L1 status of cancer cells beyond those which currently are used in the art.

### SUMMARY

The present disclosure provides computer-implemented methods of identifying programmed-death ligand 1 (PD-L1) expression status of a subject's sample comprising a cancer cell. In exemplary embodiments, the method comprises (a) receiving an unlabeled expression data set for the subject's sample and (b) aligning the unlabeled expression data set to labeled expression data according to a trained PD-L1 predictive model, wherein the trained PD-L1 predictive model has been trained with a plurality of labeled expression data sets, each labeled expression data set comprising expression data for a sample of a labeled cancer type and a labeled PD-L1 expression status, wherein aligning the unlabeled gene expression data set to labeled expression data according to the trained PD-L1 predictive model identifies PD-L1 expression status for the subject's sample.

The present disclosure also provides a method of preparing a clinical decision support information (CDSI) report. In exemplary embodiments, the method comprises (a) receiving a subject's sample, (b) identifying PD-L1 expression status of the subject's sample as determined by an alignment of an unlabeled gene expression data set of the subject's sample to labeled expression data according to a trained PD-L1 predictive model, (c) preparing a CDSI report for the subject based on the PD-L1 expression status identified in step (b), wherein the CDSI report comprises the subject's identity, the PD-L1 expression status identified in step (b), and, optionally, one or more of the date on which the sample was obtained from the subject, the sample type, a list of candidate drugs correlating with the PD-L1 expression status, data from images of the subject's tumor or cancer, image features, clinical data of the subject, epigenetic data of the subject, data from the subject's medical history and/or family history, subject's pharmacogenetic data, subject's metabolomics data, tumor mutational burden (TMB), microsatellite instability (MSI) status, estimates of immune infiltration, immunotherapy resistance mutations, estimates of the inflammatory status of the tumor microenvironment, and human leukocyte antigen (HLA) type.

A clinical decision support information (CDSI) report prepared by the presently disclosed method are further provided by the present disclosure.

Methods of determining treatment for a subject with cancer are further provided herein. In exemplary aspects, the method comprises consulting a clinical decision support information (CDSI) report of the present disclosure. In exemplary aspects, the treatment is an immune checkpoint blockade therapy comprising treatment with one or more of ipilimumab, nivolumab, pembrolizumab, atezolizumab, avelumab, durvalumab.

Computing devices configured to identify programmed-death ligand 1 (PD-L1) expression status of a subject's sample comprising a cancer cell, are further provided herein. In exemplary aspects, the computing device comprises one or more processors configured to: receive an unlabeled expression data set for the subject's sample; align the unlabeled expression data set to labeled expression data according to a trained PD-L1 predictive model, wherein the trained predictive model is trained with a plurality of labeled expression data sets, each labeled expression data set comprising expression data for a sample of a labeled cancer type and a labeled PD-L1 expression status; and predict PD-L1 expression status for the subject's sample from the alignment of the unlabeled gene expression data set to labeled expression data according to the trained PD-L1 predictive model.

### BRIEF DESCRIPTION OF THE DRAWINGS

FIG. 1A is a block diagram of a system configured to perform various methods and techniques herein, including identifying programmed-death ligand 1 (PD-L1) from genetic expression data, in accordance with an example.

FIG. 1B is a block diagram of an example portion of memory of the system of FIG. 1B storing test patient data stores, in accordance with an example.

FIG. 2A illustrates an example molecular and clinical data analysis workflow to generate a clinical report using the system of FIG. 1A, in accordance with an example.

FIG. 2B illustrates a schematic of a distributed diagnostic environment having processing devices for performing various methods and techniques herein, including the example molecular and clinical data analysis workflow of FIG. 2A, in accordance with an example.

FIG. 3 illustrates an example nucleic acid sequencing workflow as may be performed using the diagnostic environment of FIG. 2B, in accordance with an example.

FIG. 4A illustrates an example bioinformatics pipeline process that may be used for feature extraction in the nucleic acid sequencing workflow of FIG. 3, in accordance with an example.

FIG. 4B illustrates a workflow for jointly analyzing a liquid biopsy sample sequence data and a normal tissue sample sequence data for matches as may be performed by the bioinformatics pipeline of FIG. 4A, in accordance with an example.

FIG. 5A illustrates an example process of PD-L1 status prediction, in accordance with an example.

FIG. 5B illustrates an exemplary predicted PD-L1 status as it may appear on a generated report, in accordance with example.

FIG. 6A illustrates a method for training a PD-L1 status predictor and predicting a PD-L1 status using a PD-L1 status predictor, in accordance with an example.

FIG. 6B illustrates an exemplary method for selecting features for a PD-L1 status predictor, in accordance with an example.

FIG. 6C illustrates an analysis used to select thresholds for classifying a PD-L1 prediction probability as negative, equivocal, or positive, in accordance with an example.

FIG. 7 illustrates example labeled data that may be included in a training data set, in accordance with an example.

FIG. 8 illustrates an example PD-L1 status predictor, in accordance with an example.

FIG. 9 illustrates a genetic sequence analysis system, in accordance with an example, in accordance with an example.

### DETAILED DESCRIPTION

### Definitions

As used herein, an “effective amount” or “therapeutically effective amount” is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the therapeutic agent being administered.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the treatment determined by the methods of the present disclosure can provide any amount or any level of treatment. Furthermore, the treatment can include treatment of one or more conditions or symptoms or signs of the cancer being treated. The treatment can encompass slowing the progression of the cancer. For example, the treatment can treat cancer by virtue of enhancing the T cell activity or an immune response against the cancer, reducing tumor or cancer growth or tumor burden, reducing metastasis of tumor cells, increasing cell death of tumor or cancer cells or increasing tumor regression, and the like. In accordance with the foregoing, provided herein are methods of determining treatment for reducing tumor growth or tumor burden or increasing tumor regression in a subject. Also, provided herein are methods of determining treatment for enhancing T cell activity or an immune response against a cancer. In exemplary embodiments, the treatment is an immune checkpoint blockade therapy, e.g., a therapy comprising treatment with one or more of ipilimumab, nivolumab, pembrolizumab, atezolizumab, avelumab, durvalumab, and the subject's CDSI report indicates a positive PD-L1 expression status.

In various aspects, the treatment treats by way of delaying the onset or recurrence of the cancer by at least 1 day, 2 days, 4 days, 6 days, 8 days, 10 days, 15 days, 30 days, two months, 3 months, 4 months, 6 months, 1 year, 2 years, 3 years, 4 years, or more. In various aspects, the methods treat by way increasing the survival of the subject. In exemplary aspects, the treatment provides therapy by way of delaying the occurrence or onset of a metastasis. In various instances, the treatment provides therapy by way of delaying the occurrence or onset of a new metastasis. Accordingly, the treatment determined by the presently disclosed methods can treat by way of delaying the occurrence or onset of a metastasis in a subject with cancer.

Although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

### Digital and Laboratory Health Care Platform:

In some embodiments, the methods and systems described herein are utilized in combination with, or as part of, a digital and laboratory health care platform that is generally targeted to medical care and research. It should be understood that many uses of the methods and systems described above, in combination with such a platform, are possible. One example of such a platform is described in U.S. patent application Ser. No. 16/657,804, filed Oct. 18, 2019, which is hereby incorporated herein by reference in its entirety for all purposes.

For example, an implementation of one or more embodiments of the methods and systems as described above may include microservices constituting a digital and laboratory health care platform supporting analysis of PD-L1 status in a cancer specimen to provide clinical support for personalized cancer therapy. Embodiments may include a single microservice for executing and delivering analysis of PD-L1 status to clinical support for personalized cancer therapy or may include a plurality of microservices each having a particular role, which together implement one or more of the embodiments above. In one example, a first microservice may execute PD-L1 analysis in order to deliver PD-L1 features to a second microservice for curating clinical support for personalized cancer therapy based on the identified features. Similarly, the second microservice may execute therapeutic analysis of the curated clinical support to deliver recommended therapeutic modalities, according to various embodiments described herein.

Where embodiments above are executed in one or more microservices with or as part of a digital and laboratory health care platform, one or more of such microservices may be part of an order management system that orchestrates the sequence of events as needed at the appropriate time and in the appropriate order necessary to instantiate embodiments above. A microservices-based order management system is disclosed, for example, in U.S. Prov. Patent Application No. 62/873,693, filed Jul. 12, 2019, which is hereby incorporated herein by reference in its entirety for all purposes.

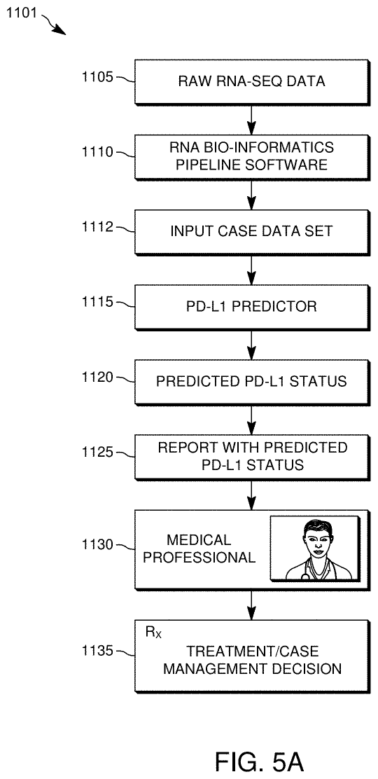

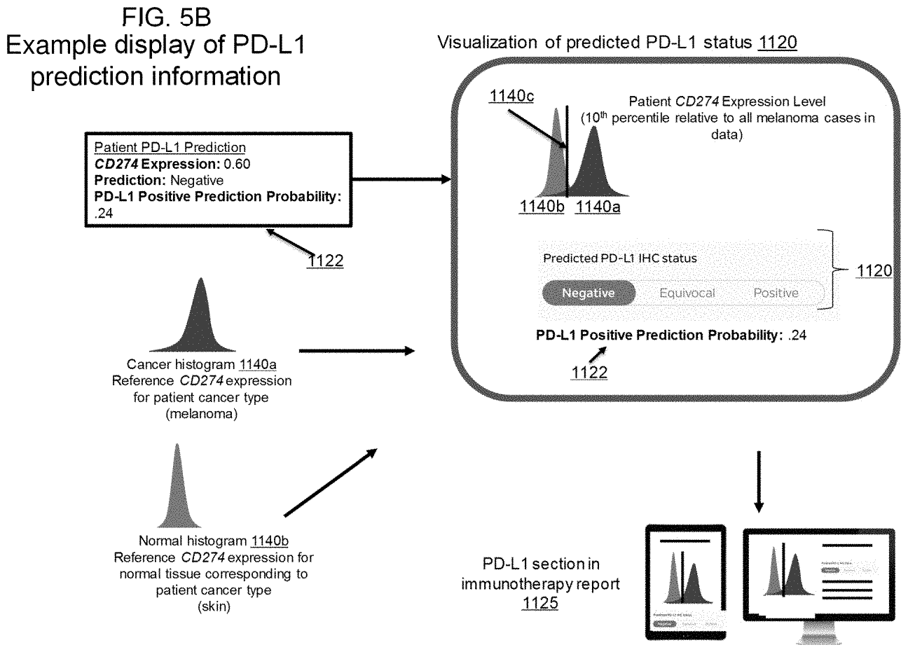

For example, continuing with the above first and second microservices, an order management system may notify the first microservice that an order for curating clinical support for personalized cancer therapy has been received and is ready for processing. The first microservice may execute and notify the order management system once the delivery of PD-L1 features for the patient is ready for the second microservice. Furthermore, the order management system may identify that execution parameters (prerequisites) for the second microservice are satisfied, including that the first microservice has completed, and notify the second microservice that it may continue processing the order to curate clinical support for personalized cancer therapy, according to various embodiments described herein.

Where the digital and laboratory health care platform further includes a genetic analyzer system, the genetic analyzer system may include targeted panels and/or sequencing probes. An example of a targeted panel is disclosed, for example, in U.S. Prov. Patent Application No. 62/902,950, filed Sep. 19, 2019, which is incorporated herein by reference and in its entirety for all purposes. In one example, targeted panels may enable the delivery of next generation sequencing results for providing clinical support for personalized cancer therapy according to various embodiments described herein. An example of the design of next-generation sequencing probes is disclosed, for example, in U.S. Prov. Patent Application No. 62/924,073, filed Oct. 21, 2019, which is incorporated herein by reference and in its entirety for all purposes.

Where the digital and laboratory health care platform further includes a bioinformatics pipeline (for example, bioinformatics pipeline 1110 in FIG. 5A), the methods and systems described above may be utilized after completion or substantial completion of the systems and methods utilized in the bioinformatics pipeline. As one example, the bioinformatics pipeline may receive next-generation genetic sequencing results and return a set of binary files, such as one or more BAM files, reflecting nucleic acid (e.g., cfDNA, DNA and/or RNA) read counts aligned to a reference genome. The methods and systems described above may be utilized, for example, to ingest the cfDNA, DNA and/or RNA read counts and produce genomic features as a result.

When the digital and laboratory health care platform further includes an RNA data normalizer, any RNA read counts may be normalized before processing embodiments as described above. An example of an RNA data normalizer is disclosed, for example, in U.S. patent application Ser. No. 16/581,706, filed Sep. 24, 2019, which is incorporated herein by reference and in its entirety for all purposes.

When the digital and laboratory health care platform further includes a genetic data deconvoluter, any system and method for deconvoluting may be utilized for analyzing genetic data associated with a specimen having two or more biological components to determine the contribution of each component to the genetic data and/or determine what genetic data would be associated with any component of the specimen if it were purified. An example of a genetic data deconvoluter is disclosed, for example, in U.S. patent application Ser. No. 16/732,229 and PCT/US19/69161, filed Dec. 31, 2019, U.S. Prov. Patent Application No. 62/924,054, filed Oct. 21, 2019, and U.S. Prov. Patent Application No. 62/944,995, filed Dec. 6, 2019, each of which is hereby incorporated herein by reference and in its entirety for all purposes.

When the digital and laboratory health care platform further includes an automated RNA expression caller, RNA expression levels may be adjusted to be expressed as a value relative to a reference expression level, which is often done in order to prepare multiple RNA expression data sets for analysis to avoid artifacts caused when the data sets have differences because they have not been generated by using the same methods, equipment, and/or reagents. An example of an automated RNA expression caller is disclosed, for example, in U.S. Prov. Patent Application No. 62/943,712, filed Dec. 4, 2019, which is incorporated herein by reference and in its entirety for all purposes.

The digital and laboratory health care platform may further include one or more insight engines to deliver information, characteristics, or determinations related to a disease state that may be based on genetic and/or clinical data associated with a patient and/or specimen. Exemplary insight engines may include a tumor of unknown origin engine, a human leukocyte antigen (HLA) loss of homozygosity (LOH) engine, a tumor mutational burden engine, a PD-L1 status engine, a homologous recombination deficiency engine, a cellular pathway activation report engine, an immune infiltration engine, a microsatellite instability engine, a pathogen infection status engine, and so forth. An example tumor of unknown origin engine is disclosed, for example, in U.S. Prov. Patent Application No. 62/855,750, filed May 31, 2019, which is incorporated herein by reference and in its entirety for all purposes. An example of an HLA LOH engine is disclosed, for example, in U.S. Prov. Patent Application No. 62/889,510, filed Aug. 20, 2019, which is incorporated herein by reference and in its entirety for all purposes. An example of a tumor mutational burden (TMB) engine is disclosed, for example, in U.S. Prov. Patent Application No. 62/804,458, filed Feb. 12, 2019, which is incorporated herein by reference and in its entirety for all purposes. An example of a PD-L1 status engine is disclosed, for example, in U.S. Prov. Patent Application No. 62/854,400, filed May 30, 2019, which is incorporated herein by reference and in its entirety for all purposes. An additional example of a PD-L1 status engine is disclosed, for example, in U.S. Prov. Patent Application No. 62/824,039, filed Mar. 26, 2019, which is incorporated herein by reference and in its entirety for all purposes. An example of a homologous recombination deficiency engine is disclosed, for example, in U.S. Prov. Patent Application No. 62/804,730, filed Feb. 12, 2019, which is incorporated herein by reference and in its entirety for all purposes. An example of a cellular pathway activation report engine is disclosed, for example, in U.S. Prov. Patent Application No. 62/888,163, filed Aug. 16, 2019, which is incorporated herein by reference and in its entirety for all purposes. An example of an immune infiltration engine is disclosed, for example, in U.S. patent application Ser. No. 16/533,676, filed Aug. 6, 2019, which is incorporated herein by reference and in its entirety for all purposes. An additional example of an immune infiltration engine is disclosed, for example, in U.S. Patent Application No. 62/804,509, filed Feb. 12, 2019, which is incorporated herein by reference and in its entirety for all purposes. An example of an MSI engine is disclosed, for example, in U.S. patent application Ser. No. 16/653,868, filed Oct. 15, 2019, which is incorporated herein by reference and in its entirety for all purposes. An additional example of an MSI engine is disclosed, for example, in U.S. Prov. Patent Application No. 62/931,600, filed Nov. 6, 2019, which is incorporated herein by reference and in its entirety for all purposes.

When the digital and laboratory health care platform further includes a report generation engine, the methods and systems described above may be utilized to create a summary report of a patient's genetic profile and the results of one or more insight engines for presentation to a physician. For instance, the report may provide to the physician information about the extent to which the specimen that was sequenced contained tumor or normal tissue from a first organ, a second organ, a third organ, and so forth. For example, the report may provide a genetic profile for each of the tissue types, tumors, or organs in the specimen. The genetic profile may represent genetic sequences present in the tissue type, tumor, or organ and may include variants, expression levels, information about gene products, or other information that could be derived from genetic analysis of a tissue, tumor, or organ. The report may include therapies and/or clinical trials matched based on a portion or all of the genetic profile or insight engine findings and summaries. For example, the therapies may be matched according to the systems and methods disclosed in U.S. Prov. Patent Application No. 62/804,724, filed Feb. 12, 2019, which is incorporated herein by reference and in its entirety for all purposes. For example, the clinical trials may be matched according to the systems and methods disclosed in U.S. Prov. Patent Application No. 62/855,913, filed May 31, 2019, which is incorporated herein by reference and in its entirety for all purposes.

The report may include a comparison of the results to a database of results from many specimens. An example of methods and systems for comparing results to a database of results are disclosed in U.S. Prov. Patent Application No. 62/786,739, filed Dec. 31, 2018, which is incorporated herein by reference and in its entirety for all purposes. The information may be used, sometimes in conjunction with similar information from additional specimens and/or clinical response information, to discover biomarkers or design a clinical trial.

When the digital and laboratory health care platform further includes application of one or more of the embodiments herein to organoids developed in connection with the platform, the methods and systems may be used to further evaluate genetic sequencing data derived from an organoid to provide information about the extent to which the organoid that was sequenced contained a first cell type, a second cell type, a third cell type, and so forth. For example, the report may provide a genetic profile for each of the cell types in the specimen. The genetic profile may represent genetic sequences present in a given cell type and may include variants, expression levels, information about gene products, or other information that could be derived from genetic analysis of a cell. The report may include therapies matched based on a portion or all of the deconvoluted information. These therapies may be tested on the organoid, derivatives of that organoid, and/or similar organoids to determine an organoid's sensitivity to those therapies. For example, organoids may be cultured and tested according to the systems and methods disclosed in U.S. patent application Ser. No. 16/693,117, filed Nov. 22, 2019; U.S. Prov. Patent Application No. 62/924,621, filed Oct. 22, 2019; and U.S. Prov. Patent Application No. 62/944,292, filed Dec. 5, 2019, each of which is incorporated herein by reference and in its entirety for all purposes.

When the digital and laboratory health care platform further includes application of one or more of the above in combination with or as part of a medical device or a laboratory developed test that is generally targeted to medical care and research, such laboratory developed test or medical device results may be enhanced and personalized through the use of artificial intelligence. An example of laboratory developed tests, especially those that may be enhanced by artificial intelligence, is disclosed, for example, in U.S. Provisional Patent Application No. 62/924,515, filed Oct. 22, 2019, which is incorporated herein by reference and in its entirety for all purposes.

It should be understood that the examples given above are illustrative and do not limit the uses of the systems and methods described herein in combination with a digital and laboratory health care platform.

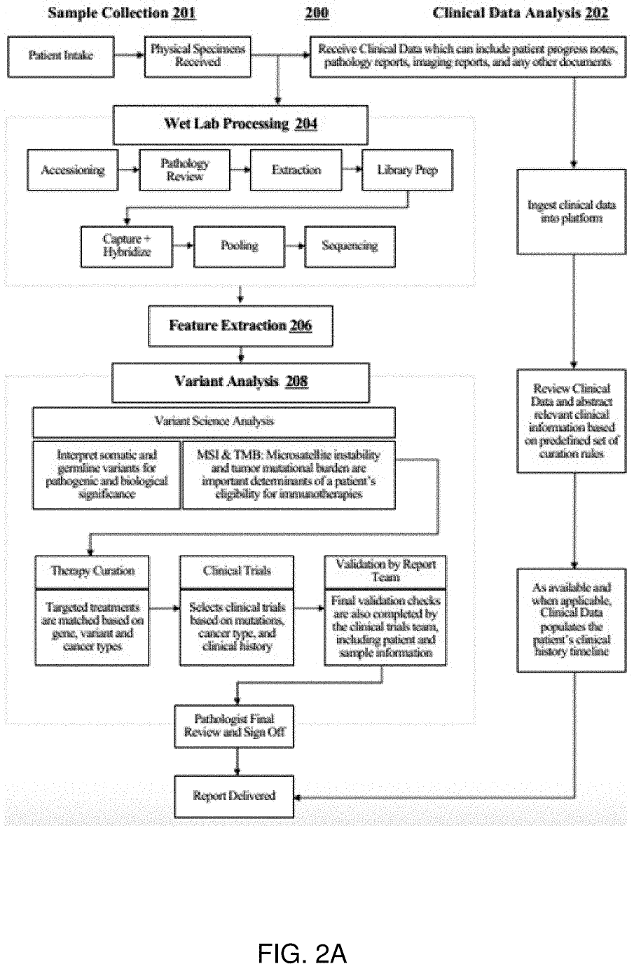

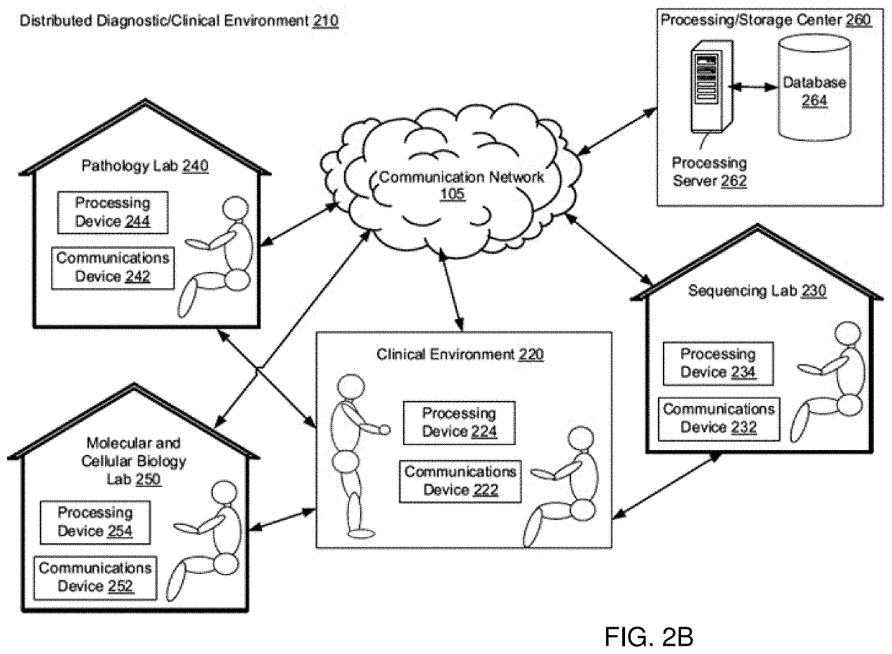

The results of the bioinformatics pipeline may be provided for report generation (e.g., variant analysis 208 described in reference to FIG. 2A). Report generation may comprise variant science analysis, including the interpretation of variants (including somatic and germline variants as applicable) for pathogenic and biological significance. The variant science analysis may also estimate microsatellite instability (MSI) or tumor mutational burden. Targeted treatments may be identified based on gene, variant, and cancer type, for further consideration and review by the ordering physician. In some aspects, clinical trials may be identified for which the patient may be eligible, based on mutations, cancer type, and/or clinical history. A validation step may occur, after which the report may be finalized for sign-out and delivery. In some embodiments, a first or second report may include additional data provided through a clinical dataflow 202, such as patient progress notes, pathology reports, imaging reports, and other relevant documents. Such clinical data is ingested, reviewed, and abstracted based on a predefined set of curation rules. The clinical data is then populated into the patient's clinical history timeline for report generation.

Further details on clinical report generation are disclosed in U.S. patent application Ser. No. 16/789,363 (PCT/US20/180002), filed Feb. 12, 2020, which is hereby incorporated herein by reference in its entirety.

### System Overview

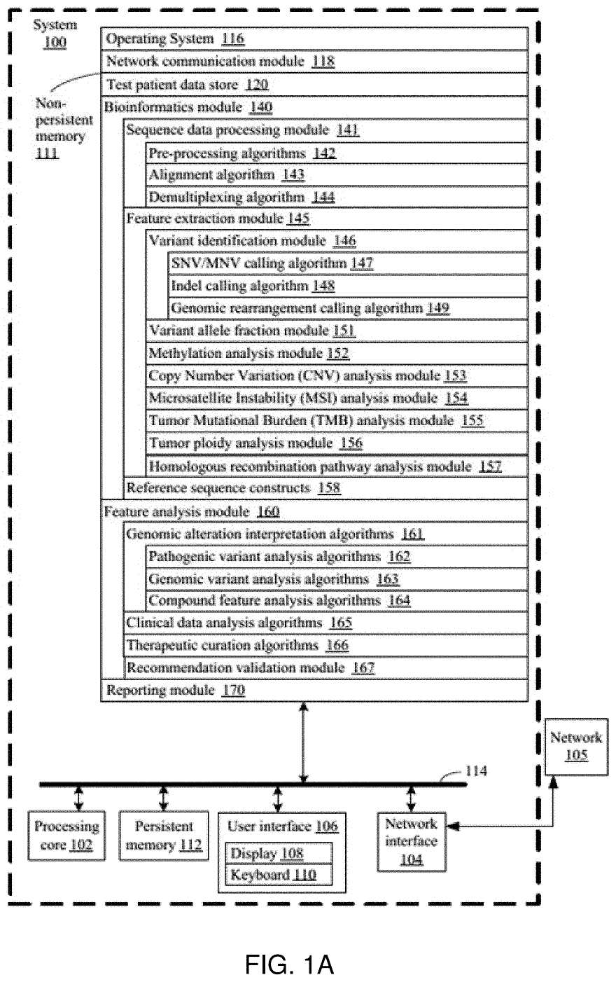

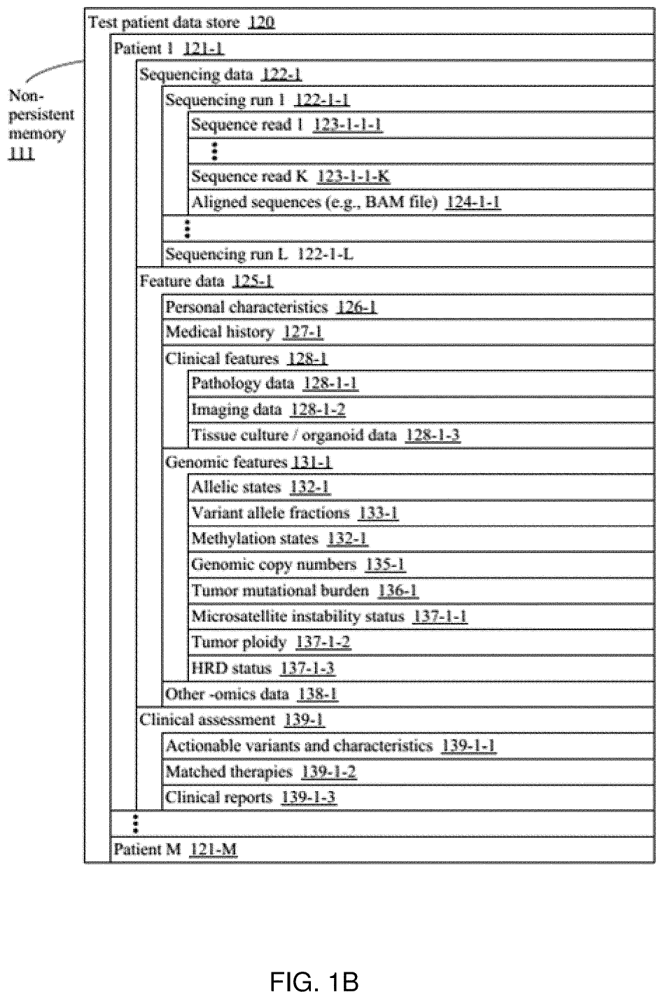

FIG. 1A is a block diagram illustrating a system in accordance with some implementations. A system 100, in some implementations, includes one or more processing units CPU(s) 102 (also referred to as processors), one or more network interfaces 104, a user interface 106, for example, including a display 108 and/or an input 110 (for example, a mouse, touchpad, keyboard, etc.), a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112: an operating system 116, which includes procedures for handling various basic system services and for performing hardware dependent tasks; a network communication module (or instructions) 118 for connecting the system 100 with other devices and/or a communication network 105; a test patient data store 120 for storing one or more collections of features from patients (for example, subjects); a bioinformatics module 140 for processing sequencing data and extracting features from sequencing data, for example, from liquid biopsy, solid tumor, or other sequencing assays, including next generation sequencing assays; a feature analysis module 160 for evaluating patient features, for example, genomic alterations, compound genomic features, and clinical features; and a reporting module 180 for generating and transmitting reports that provide clinical support for personalized cancer therapy.

Although FIGS. 1A and 1B depict a “system 100,” the figures are intended more as a functional description of the various features that may be present in computer systems than as a structural schematic of the implementations described herein. In practice, and as recognized by those of ordinary skill in the art, items shown separately could be combined and some items could be separated. Moreover, although FIGS. 1A and 1B depict certain data and modules in non-persistent memory 111, some or all of these data and modules may be in persistent memory 112. For example, in various implementations, one or more of the above identified elements are stored in one or more of the previously mentioned memory devices, and correspond to a set of instructions for performing a function described above. The above identified modules, data, or programs (for example, sets of instructions) need not be implemented as separate software programs, procedures, datasets, or modules, and thus various subsets of these modules and data may be combined or otherwise re-arranged in various implementations.

In some implementations, the non-persistent memory 111 optionally stores a subset of the modules and data structures identified above. Furthermore, in some embodiments, the memory stores additional modules and data structures not described above. In some embodiments, one or more of the above-identified elements is stored in a computer system, other than that of system 100, that is addressable by system 100 so that system 100 may retrieve all or a portion of such data when needed.

For purposes of illustration in FIG. 1A, system 100 is represented as a single computer that includes all of the functionality for providing clinical support for personalized cancer therapy. However, while a single machine is illustrated, the term “system” shall also be taken to include any collection of machines that individually or jointly execute a set (or multiple sets) of instructions to perform any one or more of the methodologies discussed herein.

For example, in some embodiments, system 100 includes one or more computers. In some embodiments, the functionality for providing clinical support for personalized cancer or other disease therapy is spread across any number of networked computers and/or resides on each of several networked computers and/or is hosted on one or more virtual machines at a remote location accessible across the communications network 105. For example, different portions of the various modules and data stores illustrated in FIGS. 1A and 1B can be stored and/or executed on the various instances of a processing device and/or processing server/database in the distributed diagnostic environment 210 illustrated in FIG. 2B (for example, processing devices 224, 234, 244, and 254, processing server 262, and database 264).

The system may operate in the capacity of a server or a client machine in client-server network environment, as a peer machine in a peer-to-peer (or distributed) network environment, or as a server or a client machine in a cloud computing infrastructure or environment. The system may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine.

In another implementation, the system comprises a virtual machine that includes a module for executing instructions for performing any one or more of the methodologies disclosed herein. In computing, a virtual machine (VM) is an emulation of a computer system that is based on computer architectures and provides functionality of a physical computer. Some such implementations may involve specialized hardware, software, or a combination of hardware and software.

One of skill in the art will appreciate that any of a wide array of different computer topologies are used for the application and all such topologies are within the scope of the present disclosure.

Referring to FIG. 1B, in some embodiments, the system (for example, system 100) includes a patient data store 120 that stores data for patients 121-1 to 121-M (for example, cancer patients or patients being tested for cancer) including one or more sequencing data 122, feature data 125, and clinical assessments 139. These data are used and/or generated by the various processes stored in the bioinformatics module 140 and feature analysis module 160 of system 100, to ultimately generate a report providing clinical support for personalized cancer therapy of a patient. While the feature scope of patient data 121 across all patients may be informationally dense, an individual patient's feature set may be sparsely populated across the entirety of the collective feature scope of all features across all patients. That is to say, the data stored for one patient may include a different set of features that the data stored for another patient. Further, while illustrated as a single data construct in FIG. 1B, different sets of patient data may be stored in different databases or modules spread across one or more system memories.

In some embodiments, sequencing data 122 from one or more sequencing reactions 122-i, including a plurality of sequence reads 123-i−1 to 123-i−K, is stored in the test patient data store 120. The data store may include different sets of sequencing data from a single subject, corresponding to different samples from the patient, for example, a tumor sample, liquid biopsy sample, tumor organoid derived from a patient tumor, and/or a normal sample, and/or to samples acquired at different times, for example, while monitoring the progression, regression, remission, and/or recurrence of a cancer in a subject. The sequence reads may be in any suitable file format, for example, BCL, FASTA, FASTQ, etc. In some embodiments, sequencing data 122 is accessed by a sequencing data processing module 141, which performs various pre-processing, genome alignment, and demultiplexing operations, as described in detail below with reference to bioinformatics module 140. In some embodiments, sequence data that has been aligned to a reference construct, for example, BAM file 124, is stored in test patient data store 120.

In some embodiments, the test patient data store 120 includes feature data 125, for example, that is useful for identifying clinical support for personalized cancer therapy. In some embodiments, the feature data 125 includes personal characteristics 126 of the patient, such as patient name, date of birth, gender, ethnicity, physical address, smoking status, alcohol consumption characteristic, anthropomorphic data, etc.

In some embodiments, the feature data 125 includes medical history data 127 for the patient, such as cancer diagnosis information (for example, date of initial diagnosis, date of metastatic diagnosis, cancer staging, tumor characterization, tissue of origin, previous treatments and outcomes, adverse effects of therapy, therapy group history, clinical trial history, previous and current medications, surgical history, etc.), previous or current symptoms, previous or current therapies, previous treatment outcomes, previous disease diagnoses, diabetes status, diagnoses of depression, diagnoses of other physical or mental maladies, and family medical history. In some embodiments, the feature data 125 includes clinical features 128, such as pathology data 128-1, medical imaging data 128-2, and tissue culture and/or tissue organoid culture data 128-3.

In some embodiments, yet other clinical features, such as previous laboratory testing results, are stored in the test patient data store 120. Medical history data 127 and clinical features may be collected from various sources, including at intake directly from the patient, from an electronic medical record (EMR) or electronic health record (EHR) for the patient, or curated from other sources, such as fields from various testing records (for example, genetic sequencing reports).

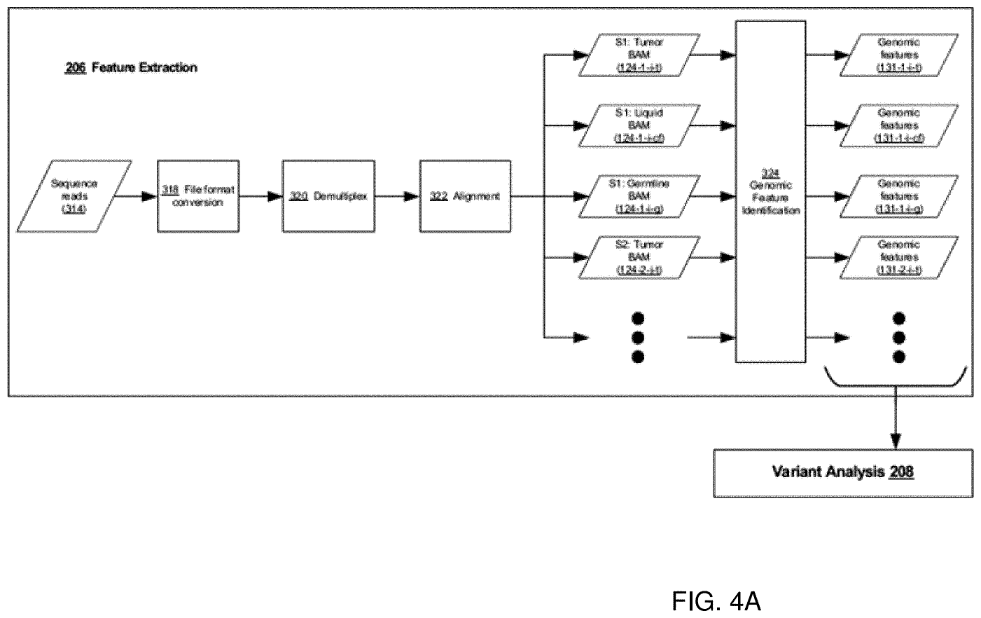

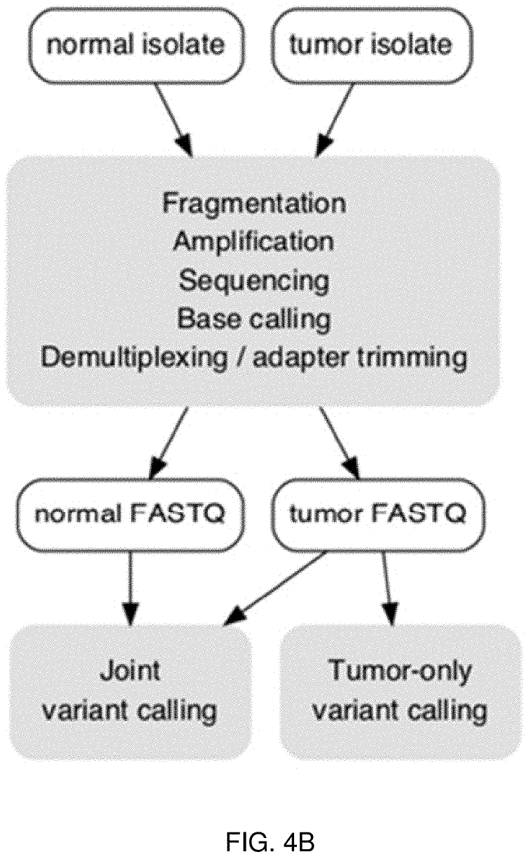

In some embodiments, the feature data 125 includes genomic features 131 for the patient. Non-limiting examples of genomic features include allelic states 132 (for example, the identity of alleles at one or more loci, support for wild type or variant alleles at one or more loci, support for SNVs/MNVs at one or more loci, support for indels at one or more loci, and/or support for gene rearrangements at one or more loci), allelic fractions 133 (for example, ratios of variant to reference alleles (or vice versa), methylation states 132 (for example, a distribution of methylation patterns at one or more loci and/or support for aberrant methylation patterns at one or more loci), genomic copy numbers 135 (for example, a copy number value at one or more loci and/or support for an aberrant (increased or decreased) copy number at one or more loci), tumor mutational burden 136 (for example, a measure of the number of mutations in the cancer genome of the subject), and microsatellite instability status 137 (for example, a measure of the repeated unit length at one or more microsatellite loci and/or a classification of the MSI status for the patient's cancer). In some embodiments, one or more of the genomic features 131 are determined by a nucleic acid bioinformatics pipeline, for example, as described in detail below with reference to FIGS. 4A & 4B. In particular, in some embodiments, the feature data 125 include fusion data, as determined using the improved methods for fusion candidate ranking, as described in further detail below. In some embodiments, one or more of the genomic features 131 are obtained from an external testing source, for example, not connected to the bioinformatics pipeline as described below.

In some embodiments, the feature data 125 further includes data 138 from other -omics fields of study. Non-limiting examples of -omics fields of study that may yield feature data useful for providing clinical support for personalized cancer therapy include transcriptomics, epigenomics, proteomics, metabolomics, metabonomics, microbiomics, lipodomics, glycomics, cellomics, and organoidomics.

In some embodiments, yet other features may include features derived from machine learning approaches, for example, based at least in part on evaluation of any relevant molecular or clinical features, considered alone or in combination, not limited to those listed above. For instance, in some embodiments, one or more latent features learned from evaluation of cancer patient training datasets improve the diagnostic and prognostic power of the various analysis algorithms in the feature analysis module 160.

The skilled artisan will know of other types of features useful for providing clinical support for personalized cancer therapy. The listing of features above is merely representative and should not be construed to be limiting.

In some embodiments, a test patient data store 120 includes clinical assessment data 139 for patients, for example, based off the feature data 125 collected for the subject. In some embodiments, the clinical assessment data 139 includes a catalogue of actionable variants and characteristics 139-1 (for example, genomic alterations and compound metrics based on genomic features known or believed to be targetable by one or more specific cancer therapies), matched therapies 139-2 (for example, the therapies known or believed to be particularly beneficial for treatment of subjects having actionable variants), and/or clinical reports 139-3 generated for the subject, for example, based on identified actionable variants and characteristics 139-1 and/or matched therapies 139-2.

In some embodiments, clinical assessment data 139 is generated by analysis of feature data 125 using the various algorithms of feature analysis module 160, as described in further detail below. In some embodiments, clinical assessment data 139 is generated, modified, and/or validated by evaluation of feature data 125 by a clinician, for example, an oncologist. For instance, in some embodiments, a clinician (for example, at clinical environment 220) uses feature analysis module 160, or accesses test patient data store 120 directly, to evaluate feature data 125 to make recommendations for personalized cancer treatment of a patient. Similarly, in some embodiments, a clinician (for example, at clinical environment 220) reviews recommendations determined using feature analysis module 160 and approves, rejects, or modifies the recommendations, for example, prior to the recommendations being sent to a medical professional treating the cancer patient.

### Genetic Sequence Data Generation

### Specimen Information

The tumor sample may be a blood sample or tissue sample containing cancer cells or circulating tumor DNA (ctDNA).

For example, a physician may perform a tumor biopsy of a patient by removing a small amount of tumor tissue/specimen from the patient and sending this specimen to a laboratory. The lab may prepare slides from the specimen using slide preparation techniques such as freezing the specimen and slicing layers, setting the specimen in paraffin and slicing layers, smearing the specimen on a slide, or other methods known to those of ordinary skill.

In some embodiments, two or more samples, slices, and/or slides are obtained from a subject—for example, two or more tissue slices can be taken that are contiguous or substantially contiguous to each other. In some cases, the tissue slices are obtained such that some of the pathology slides prepared from the respective slices are imaged (for example, histopathology slides, hematoxylin and eosin stained slides, immunohistochemistry stained slides, etc.), whereas some of the pathology slides are used for obtaining sequencing information.

In some instances, a tumor organoid sample may be processed instead of a patient tumor sample.

In more detail, germline (“normal”, non-cancerous) DNA may be extracted from either blood (for example, if a patient has cancer that is not a blood cancer) or saliva (for example, if a patient has blood cancer). Normal blood samples may be collected from patients (for example, in PAXgene Blood DNA Tubes) and saliva samples may be collected from patients (for example, in Oragene DNA Saliva Kits).

Blood cancer samples may be collected from patients (for example, in EDTA collection tubes). Macrodissected or microdissected FFPE tissue sections (which may be mounted on a histopathology slide) from solid tumor samples may be analyzed by pathologists to determine overall tumor amount in the sample and percent tumor cellularity as a ratio of tumor to normal nuclei. For each section, background tissue may be excluded or removed such that the section meets a tumor purity threshold (in one example, at least 20% of the cell nuclei in the section are tumor cell nuclei).

FIG. 2A illustrates an example molecular and clinical data analysis workflow to generate a clinical report. Briefly, the workflow begins with patient intake and sample collection 201, where one or more liquid biopsy samples, one or more tumor biopsy, and one or more normal and/or control tissue samples are collected from the patient (for example, at a clinical environment 220 or home healthcare environment). In some embodiments, personal data 126 corresponding to the patient and a record of the one or more biological samples obtained (for example, patient identifiers, patient clinical data, sample type, sample identifiers, cancer conditions, etc.) are entered into a data analysis platform, for example, test patient data store 120. Accordingly, in some embodiments, the methods disclosed herein include obtaining one or more biological samples from one or more subjects, for example, cancer patients. In some embodiments, the subject is a human, for example, a human cancer patient.

In some embodiments, one or more of the biological samples obtained from the patient are a biological liquid sample, also referred to as a liquid biopsy sample. In some embodiments, one or more of the biological samples obtained from the patient are selected from blood, plasma, serum, urine, vaginal fluid, fluid from a hydrocele (for example, of the testis), vaginal flushing fluids, pleural fluid, ascitic fluid, cerebrospinal fluid, saliva, sweat, tears, sputum, bronchioalveolar lavage fluid, discharge fluid from the nipple, aspiration fluid from different parts of the body (for example, thyroid, breast), etc. In some embodiments, the liquid biopsy sample includes blood and/or saliva. In some embodiments, the liquid biopsy sample is peripheral blood.

In some embodiments, a liquid biopsy sample is separated into two different samples. For example in some embodiments, a blood sample is separated into a blood plasma sample, containing cf DNA, and a buffy coat preparation, containing white blood cells. In some embodiments, a plurality of liquid biopsy samples is obtained from a respective subject at intervals over a period of time (for example, using serial testing).

In some embodiments, one or more biological samples collected from the patient is a solid tissue sample, for example, a solid tumor sample or a solid normal tissue sample. Methods for obtaining solid tissue samples, for example, of cancerous and/or normal tissue are known in the art, and are dependent upon the type of tissue being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (for example, fine-needle aspiration, core needle aspiration, vacuum-assisted biopsy, and image-guided biopsy, can be used to obtain samples of subdermal tumors, skin biopsies, for example, shave biopsy, punch biopsy, incisional biopsy, and excisional biopsy, can be used to obtain samples of dermal cancers, and surgical biopsies can be used to obtain samples of cancers affecting internal organs of a patient. In some embodiments, a solid tissue sample is a formalin-fixed tissue (FFT). In some embodiments, a solid tissue sample is a macro-dissected formalin fixed paraffin embedded (FFPE) tissue. In some embodiments, a solid tissue sample is a fresh frozen tissue sample.

In some embodiments, a dedicated normal sample is collected from the patient, for co-processing with a liquid biopsy or solid tissue sample. Generally, the normal sample is of a non-cancerous tissue, and can be collected using any tissue collection means described above. In some embodiments, buccal cells collected from the inside of a patient's cheeks are used as a normal sample. Buccal cells can be collected by placing an absorbent material, for example, a swab, in the subjects mouth and rubbing it against their cheek, for example, for at least 15 second or for at least 30 seconds. The swab is then removed from the patient's mouth and inserted into a tube, such that the tip of the tube is submerged into a liquid that serves to extract the buccal cells off of the absorbent material. An example of buccal cell recovery and collection devices is provided in U.S. Pat. No. 9,138,205, the content of which is hereby incorporated by reference, in its entirety, for all purposes. In some embodiments, the buccal swab DNA is used as a source of normal DNA in circulating heme malignancies.

### Probe Overview

In some embodiments, a plurality of nucleic acid probes (for example, a probe set) is used to enrich one or more target sequences in a nucleic acid sample (for example, an isolated nucleic acid sample or a nucleic acid sequencing library). Probes may be designed and created in accordance with methods known in the art. In some embodiments, the probe set includes probes targeting one or more gene loci, for example, exon or intron loci. In some embodiments, the probe set includes probes targeting one or more loci not encoding a protein, for example, regulatory loci, miRNA loci, and other non-coding loci, for example, that have been found to be associated with cancer. In some embodiments, the plurality of loci include at least 25, 50, 100, 150, 200, 250, 300, 350, 400, 500, 750, 1000, 2500, 5000, or more human genomic loci.

Generally, probes for enrichment of nucleic acids (for example, complementary DNA, cDNA, generated from nucleic acids extracted or isolated from a biological specimen, including extracted or isolated RNA) include DNA, RNA, or a modified nucleic acid structure with a base sequence that is complementary to a loci of interest. For instance, a probe designed to hybridize to a loci in a cDNA molecule can contain a sequence that is complementary to either strand, because the cDNA molecules may be double stranded. In some embodiments, each probe in the plurality of probes includes a nucleic acid sequence that is identical or complementary to at least 10, at least 11, at least 12, at least 13, at least 14, or at least 15 consecutive bases of a loci of interest. In some embodiments, each probe in the plurality of probes includes a nucleic acid sequence that is identical or complementary to at least 20, 25, 30, 40, 50, 75, 100, 150, 200, or more consecutive bases of a locus of interest.

Probes may be created in accordance with the methods set forth in FastPCR Software for PCR Primer and Probe Design and Repeat Search (Kalendar et al., 2009 Genes, Genomes, and Genomics, 3 (Special Issue 1), pp. 1-14) which is incorporated by reference herein.

Targeted-panels provide several benefits for nucleic acid sequencing. In one example, panels targeting genes with high variability among individual subjects, humans, or even cells within subjects or humans may facilitate bioinformatics processing to determine the sequences of those genes. For example, if a “whole exome” or targeted sequencing panel is not generating a sufficient number of sequencing reads mapping to the high-variable genes, probes targeting the high-variable genes may be added to the whole exome or targeted sequence panel probes to increase the number reads mapping to high-variable genes.

In some embodiments, the gene panel is a whole-exome panel that analyzes the exomes of a biological sample. In some embodiments, the gene panel is a whole-genome panel that analyzes the genome of a specimen. In some preferred embodiments, the gene panel is optimized for use with specific cells or cell types of interest. For instance, the gene panel may be optimized for use in a cancer gene panel (for example, to provide clinical decision support related to cancer treatment).

In some embodiments, the probes include additional nucleic acid sequences that do not share any homology to the loci of interest. For example, in some embodiments, the probes also include nucleic acid sequences containing an identifier sequence, for example, a unique molecular identifier (UMI), for example, that is unique to a particular sample or subject. Examples of identifier sequences are described, for example, in Kivioja et al., 2011, Nat. Methods 9(1), pp. 72-74 and Islam et al., 2014, Nat. Methods 11(2), pp. 163-66, which are incorporated by reference herein. Similarly, in some embodiments, the probes also include primer nucleic acid sequences useful for amplifying the nucleic acid molecule of interest, for example, using PCR. In some embodiments, the probes also include a capture sequence designed to hybridize to an anti-capture sequence for recovering the nucleic acid molecule of interest from the sample.

Likewise, in some embodiments, the probes each include a non-nucleic acid affinity moiety covalently attached to nucleic acid molecule that is complementary to the loci of interest, for recovering the nucleic acid molecule of interest. Non-limited examples of non-nucleic acid affinity moieties include biotin, digoxigenin, and dinitrophenol. In some embodiments, the probe is attached to a solid-state surface or particle, for example, a dip-stick or magnetic bead, for recovering the nucleic acid of interest. In some embodiments, the methods described herein include amplifying the nucleic acids that bound to the probe set prior to further analysis, for example, sequencing. Methods for amplifying nucleic acids, for example, by PCR, are well known in the art.

### Probe Concentration.

In some embodiments, probes may be included as part of a comprehensive genomic profiling panel. Examples include a whole exome RNAseq panel, a targeted enrichment sequencing panel, a whole-exome panel, a whole genome panel, etc.

Probes may be separated into various pools. The concentration of each group (pool) of probes may be adjusted to achieve desired coverage. The concentration of each pool may be adjusted in accordance with, for example, the systems and methods disclosed in U.S. Prov. Patent App. No. 62/924,073, titled “Systems and Methods for Next Generation Sequencing Probe Design”, filed Oct. 21, 2019 and incorporated by reference herein in its entirety.

### DNA Profiling

In various embodiments, each DNA data set may be generated by processing a cancer sample and a non-cancer sample from the same patient, or only a cancer sample through DNA next generation sequencing (NGS), designed to sequence either the whole exome or a targeted panel of cancer-related genes, to generate DNA sequencing data. The cancer sample may be tissue, blood, or cell-free, circulating tumor DNA. The DNA sequencing data may be processed by a bioinformatics pipeline to generate a DNA variant call file (among other outputs) for each sample.

The biological samples collected from the patient are, optionally, sent to various analytical environments (for example, sequencing lab 230, pathology lab 240, and/or molecular biology lab 250) for processing (for example, data collection) and/or analysis (for example, feature extraction). Wet lab processing 204 may include the steps of cataloguing samples (for example, accessioning), examining clinical features of one or more samples (for example, pathology review), and nucleic acid sequence analysis (for example, extraction, library prep, capture+hybridize, pooling, and sequencing). In some embodiments, the workflow includes clinical analysis of one or more biological samples collected from the subject, for example, at a pathology lab 240 and/or a molecular and cellular biology lab 250, to generate clinical features such as pathology features 128-3, imaging data 128-3, and/or tissue culture/organoid data 128-3.

In some embodiments, the pathology data 128-1 collected during clinical evaluation includes visual features identified by a pathologist's inspection of a specimen (for example, a solid tumor biopsy), for example, of stained H&E or IHC slides. In some embodiments, the sample is a solid tissue biopsy sample. In some embodiments, the tissue biopsy sample is a formalin-fixed tissue (FFT), for example, a formalin-fixed paraffin-embedded (FFPE) tissue. In some embodiments, the tissue biopsy sample is an FFPE or FFT block. In some embodiments, the tissue biopsy sample is a fresh-frozen tissue biopsy. The tissue biopsy sample can be prepared in thin sections (for example, by cutting and/or affixing to a slide), to facilitate pathology review (for example, by staining with immunohistochemistry stain for IHC review and/or with hematoxylin and eosin stain for H&E pathology review). For instance, analysis of slides for H&E staining or IHC staining may reveal features such as tumor infiltration, programmed death-ligand 1 (PD-L1) status, human leukocyte antigen (HLA) status, or other immunological features.

In some embodiments, a liquid sample (for example, blood) collected from the patient (for example, in EDTA-containing collection tubes) is prepared on a slide (for example, by smearing) for pathology review. In some embodiments, macrodissected FFPE tissue sections, which may be mounted on a histopathology slide, from solid tissue samples (for example, tumor or normal tissue) are analyzed by pathologists. In some embodiments, tumor samples are evaluated to determine, for example, the tumor purity of the sample, the percent tumor cellularity as a ratio of tumor to normal nuclei, etc. For each section, background tissue may be excluded or removed such that the section meets a tumor purity threshold, for example, where at least 20% of the nuclei in the section are tumor nuclei, or where at least 25%, 30%, 40%, 50%, 60%, 70%, 80%, 90%, or more of the nuclei in the section are tumor nuclei.

In some embodiments, pathology data 128-1 is extracted, in addition to or instead of visual inspection, using computational approaches to digital pathology, for example, providing morphometric features extracted from digital images of stained tissue samples. A review of digital pathology methods is provided in Bera, K. et al., Nat. Rev. Clin. Oncol., 16:703-15 (2019), the content of which is hereby incorporated by reference, in its entirety, for all purposes. In some embodiments, pathology data 128-1 includes features determined using machine learning algorithms to evaluate pathology data collected as described above.

In some embodiments, imaging data 128-2 collected during clinical evaluation includes features identified by review of in-vitro and/or in-vivo imaging results (for example, of a tumor site), for example a size of a tumor, tumor size differentials over time (such as during treatment or during other periods of change). In some embodiments, imaging data 128-2 includes features determined using machine learning algorithms to evaluate imaging data collected as described above.

In some embodiments, tissue culture/organoid data 128-3 collected during clinical evaluation includes features identified by evaluation of cultured tissue from the subject. For instance, in some embodiments, tissue samples obtained from the patients (for example, tumor tissue, normal tissue, or both) are cultured (for example, in liquid culture, solid-phase culture, and/or organoid culture) and various features, such as cell morphology, growth characteristics, genomic alterations, and/or drug sensitivity, are evaluated. In some embodiments, tissue culture/organoid data 128-3 includes features determined using machine learning algorithms to evaluate tissue culture/organoid data collected as described above. Examples of tissue organoid (for example, personal tumor organoid) culturing and feature extractions thereof are described in U.S. Provisional Application Ser. No. 62/924,621, filed on Oct. 22, 2019, and U.S. patent application Ser. No. 16/693,117, filed on Nov. 22, 2019, the contents of which are hereby incorporated by reference, in their entireties, for all purposes.

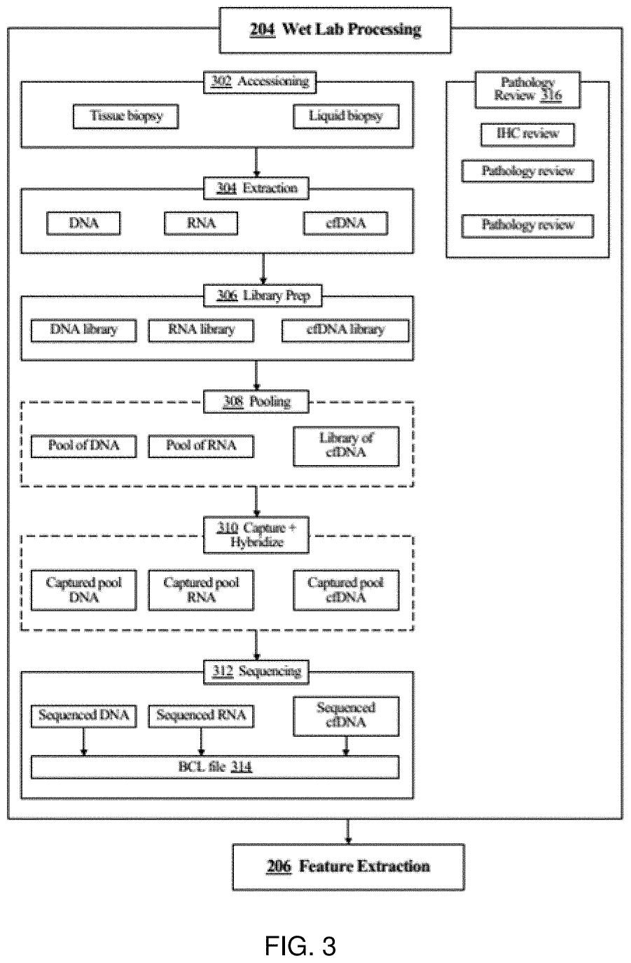

Nucleic acid sequencing of one or more samples collected from the subject is performed, for example, at sequencing lab 230, during wet lab processing 204. An example workflow for nucleic acid sequencing is illustrated in FIG. 3. In some embodiments, the one or more biological samples obtained at the sequencing lab 230 are accessioned (see, 302 in FIG. 3), to track the sample and data through the sequencing process.

Next, nucleic acids, for example, RNA and/or DNA are extracted (see, 304 in FIG. 3) from the one or more biological samples. Methods for isolating nucleic acids from biological samples are known in the art, and are dependent upon the type of nucleic acid being isolated (for example, cfDNA, DNA, and/or RNA) and the type of sample from which the nucleic acids are being isolated (for example, liquid biopsy samples, white blood cell buffy coat preparations, formalin-fixed paraffin-embedded (FFPE) solid tissue samples, and fresh frozen solid tissue samples). The selection of any particular nucleic acid isolation technique for use in conjunction with the embodiments described herein is well within the skill of the person having ordinary skill in the art, who will consider the sample type, the state of the sample, the type of nucleic acid being sequenced and the sequencing technology being used.

For instance, many techniques for DNA isolation, for example, genomic DNA isolation, from a tissue sample are known in the art, such as organic extraction, silica adsorption, and anion exchange chromatography. Likewise, many techniques for RNA isolation, for example, mRNA isolation, from a tissue sample are known in the art. For example, acid guanidinium thiocyanate-phenol-chloroform extraction (see, for example, Chomczynski and Sacchi, 2006, Nat Protoc, 1(2):581-85, which is hereby incorporated by reference herein), and silica bead/glass fiber adsorption (see, for example, Poeckh, T. et al., 2008, Anal Biochem., 373(2):253-62, which is hereby incorporated by reference herein). The selection of any particular DNA or RNA isolation technique for use in conjunction with the embodiments described herein is well within the skill of the person having ordinary skill in the art, who will consider the tissue type, the state of the tissue, for example, fresh, frozen, formalin-fixed, paraffin-embedded (FFPE), and the type of nucleic acid analysis that is to be performed.

In some embodiments where the biological sample is a liquid biopsy sample, for example, a blood or blood plasma sample, cfDNA is isolated from blood samples using commercially available reagents, including proteinase K, to generate a liquid solution of cf DNA.

Additionally, DNA may be isolated from cells in blood samples, saliva samples, and tissue sections by lysing cells and using commercially available reagents, including proteinase K to generate a liquid solution of DNA.

In some embodiments, isolated DNA or cf DNA molecules are mechanically sheared to an average length using an ultrasonicator (for example, a Covaris ultrasonicator). In some embodiments, isolated nucleic acid molecules are analyzed to determine their fragment size, for example, through gel electrophoresis techniques and/or the use of a device such as a LabChip GX Touch.

In some embodiments, quality control testing is performed on the extracted nucleic acids (for example, DNA and/or RNA), for example, to assess the nucleic acid concentration and/or fragment size. For example, sizing of DNA fragments provides valuable information used for downstream processing, such as determining whether DNA fragments require additional shearing prior to sequencing.

Wet lab processing then includes preparing a nucleic acid library from the isolated nucleic acids (for example, cfDNA, DNA, and/or RNA, see, e.g., 306 in FIG. 3). For example, in some embodiments, DNA libraries (for example, gDNA and/or cf DNA libraries) are prepared from isolated DNA from the one or more biological samples. In some embodiments, the DNA libraries are prepared using a commercial library preparation kit, for example, the KAPA Hyper Prep Kit, a New England Biolabs (NEB) kit, or a similar kit.

In some embodiments, during library preparation, adapters (for example, UDI adapters, such as Roche SeqCap dual end adapters, or UMI adapters such as full length or stubby Y adapters) are ligated onto the nucleic acid molecules. In some embodiments, the adapters include unique molecular identifiers (UMIs), which are short nucleic acid sequences (for example, 4-10 base pairs) that are added to ends of DNA fragments during adapter ligation. In some embodiments, UMIs are degenerate base pairs that serve as a unique tag that can be used to identify sequence reads originating from a specific DNA fragment. In some embodiments, for example, when multiplex sequencing will be used to sequence DNA from a plurality of samples (for example, from the same or different subjects) in a single sequencing reaction, a patient-specific index is also added to the nucleic acid molecules. In some embodiments, the patient specific index is a short nucleic acid sequence (for example, 3-20 nucleotides) that are added to ends of DNA fragments during library construction, that serve as a unique tag that can be used to identify sequence reads originating from a specific patient sample. Examples of identifier sequences are described, for example, in Kivioja et al., Nat. Methods 9(1):72-74 (2011) and Islam et al., Nat. Methods 11(2):163-66 (2014), the contents of which are hereby incorporated by reference, in their entireties, for all purposes.

In some embodiments, an adapter includes a PCR primer landing site, designed for efficient binding of a PCR or second-strand synthesis primer used during the sequencing reaction. In some embodiments, an adapter includes an anchor binding site, to facilitate binding of the DNA molecule to anchor oligonucleotide molecules on a sequencer flow cell, serving as a seed for the sequencing process by providing a starting point for the sequencing reaction. During PCR amplification following adapter ligation, the UMIs, patient indexes, and binding sites are replicated along with the attached DNA fragment. This provides a way to identify sequence reads that came from the same original fragment in downstream analysis.

In some embodiments, DNA libraries are amplified and purified using commercial reagents, (for example, Axygen MAG PCR clean up beads). In some such embodiments, the concentration and/or quantity of the DNA molecules are then quantified using a fluorescent dye and a fluorescence microplate reader, standard spectrofluorometer, or filter fluorometer. In some embodiments, library amplification is performed on a device (for example, an Illumina C-Bot2) and the resulting flow cell containing amplified target-captured DNA libraries is sequenced on a next generation sequencer (for example, an Illumina HiSeq 4000 or an Illumina NovaSeq 6000) to a unique on-target depth selected by the user. In some embodiments, DNA library preparation is performed with an automated system, using a liquid handling robot (for example, a SciClone NGSx).

In some embodiments, wet lab processing 204 includes pooling (see, 308 in FIG. 3) DNA molecules from a plurality of libraries, corresponding to different samples from the same and/or different patients, to forming a sequencing pool of DNA libraries. When the pool of DNA libraries is sequenced, the resulting sequence reads correspond to nucleic acids isolated from multiple samples. The sequence reads can be separated into different sequence read files, corresponding to the various samples represented in the sequencing read based on the unique identifiers present in the added nucleic acid fragments. In this fashion, a single sequencing reaction can generate sequence reads from multiple samples. Advantageously, this allows for the processing of more samples per sequencing reaction.

In some embodiments, wet lab processing 204 includes enriching (see, 310 in FIG. 3) a sequencing library, or pool of sequencing libraries, for target nucleic acids, for example, nucleic acids encompassing loci that are informative for precision oncology and/or used as internal controls for the sequencing or bioinformatics processes. In some embodiments, enrichment is achieved by hybridizing target nucleic acids in the sequencing library to probes that hybridize to the target sequences, and then isolating the captured nucleic acids away from off-target nucleic acids that are not bound by the capture probes.

Advantageously, enriching for target sequences prior to sequencing nucleic acids significantly reduces the costs and time associated with sequencing, facilitates multiplex sequencing by allowing multiple samples to be mixed together for a single sequencing reaction, and significantly reduces the computation burden of aligning the resulting sequence reads, as a result of significantly reducing the total amount of nucleic acids analyzed from each sample.

In some embodiments, the enrichment is performed prior to pooling multiple nucleic acid sequencing libraries. However, in other embodiments, the enrichment is performed after pooling nucleic acid sequencing libraries, which has the advantage of reducing the number of enrichment assays that have to be performed.

In some embodiments, the enrichment is performed prior to generating a nucleic acid sequencing library. This has the advantage that fewer reagents are needed to perform both the enrichment (because there are fewer target sequences at this point, prior to library amplification) and the library production (because there are fewer nucleic acid molecules to tag and amplify after the enrichment). However, this raises the possibility of pull-down bias and/or that small variations in the enrichment protocol will result in less consistent results.

In some embodiments, nucleic acid libraries are pooled (two or more DNA libraries may be mixed to create a pool) and treated with reagents to reduce off-target capture, for example Human COT-1 and/or IDT xGen Universal Blockers. Pools may be dried in a vacufuge and resuspended. DNA libraries or pools may be hybridized to a probe set (for example, a probe set specific to a panel that includes loci from at least 100, 600, 1,000, 10,000, etc. of the 19,000 known human genes) and amplified with commercially available reagents (for example, the KAPA HiFi HotStart ReadyMix). For example, in some embodiments, a pool is incubated in an incubator, PCR machine, water bath, or other temperature-modulating device to allow probes to hybridize. Pools may then be mixed with Streptavidin-coated beads or another means for capturing hybridized DNA-probe molecules, such as DNA molecules representing exons of the human genome and/or genes selected for a genetic panel.

Pools may be amplified and purified more than once using commercially available reagents, for example, the KAPA HiFi Library Amplification kit and Axygen MAG PCR clean up beads, respectively. The pools or DNA libraries may be analyzed to determine the concentration or quantity of DNA molecules, for example by using a fluorescent dye (for example, PicoGreen pool quantification) and a fluorescence microplate reader, standard spectrofluorometer, or filter fluorometer. In one example, the DNA library preparation and/or capture steps may be performed with an automated system, using a liquid handling robot (for example, a SciClone NGSx).

Sequence reads are then generated (see, 312 in FIG. 3) from the sequencing library or pool of sequencing libraries. Sequencing data may be acquired by any methodology known in the art. For example, next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLiD sequencing), nanopore sequencing (Oxford Nanopore Technologies), or paired-end sequencing. In some embodiments, massively parallel sequencing is performed using sequencing-by-synthesis with reversible dye terminators. In some embodiments, sequencing is performed using next generation sequencing technologies, such as short-read technologies. In other embodiments, long-read sequencing or another sequencing method known in the art is used.

Next-generation sequencing produces millions of short reads (for example, sequence reads) for each biological sample. Accordingly, in some embodiments, the plurality of sequence reads obtained by next-generation sequencing of cfDNA molecules are DNA sequence reads. In some embodiments, the sequence reads have an average length of at least fifty nucleotides. In other embodiments, the sequence reads have an average length of at least 50, 60, 70, 80, 90, 100, 150, 200, 250, 300, or more nucleotides.

In some embodiments, sequencing is performed after enriching for nucleic acids (for example, cf DNA, gDNA, and/or RNA) encompassing a plurality of predetermined target sequences, for example, human genes and/or non-coding sequences associated with cancer. Advantageously, sequencing a nucleic acid sample that has been enriched for target nucleic acids, rather than all nucleic acids isolated from a biological sample, significantly reduces the average time and cost of the sequencing reaction. Accordingly, in some preferred embodiments, the methods described herein include obtaining a plurality of sequence reads of nucleic acids that have been hybridized to a probe set for hybrid-capture enrichment.

In some embodiments, panel-targeting sequencing is performed to an average on-target depth of at least 500×, at least 750×, at least 1000×, at least 2500×, at least 500×, at least 10,000×, or greater depth. In some embodiments, samples are further assessed for uniformity above a sequencing depth threshold (for example, 95% of all targeted base pairs at 300× sequencing depth). In some embodiments, the sequencing depth threshold is a minimum depth selected by a user or practitioner.

In some embodiments, the sequence reads are obtained by a whole genome or whole exome sequencing methodology. In some such embodiments, whole exome capture steps may be performed with an automated system, using a liquid handling robot (for example, a SciClone NGSx).

In some embodiments, the raw sequence reads resulting from the sequencing reaction are output from the sequencer in a native file format, for example, a BCL file (see, 314 in FIG. 3). In some embodiments, the native file is passed directly to a bioinformatics pipeline (for example, variant analysis 206), components of which are described in detail below. In other embodiments, one or more pre-processing steps are performed prior to passing the sequences to the bioinformatics platform. For instance, in some embodiments, the format of the sequence read file is converted from the native file format (for example, BCL) to a file format compatible with one or more algorithms used in the bioinformatics pipeline (for example, FASTQ or FASTA). In some embodiments, the raw sequence reads are filtered to remove sequences that do not meet one or more quality thresholds. In some embodiments, raw sequence reads generated from the same unique nucleic acid molecule in the sequencing read are collapsed into a single sequence read representing the molecule, for example, using UMIs as described above. In some embodiments, one or more of these pre-processing steps are performed within the bioinformatics pipeline itself.

In one example, a sequencer may generate a BCL file. A BCL file may include raw image data of a plurality of patient specimens which are sequenced. BCL image data is an image of the flow cell across each cycle during sequencing. A cycle may be implemented by illuminating a patient specimen with a specific wavelength of electromagnetic radiation, generating a plurality of images which may be processed into base calls via BCL to FASTQ processing algorithms which identify which base pairs are present at each cycle. The resulting FASTQ file includes the entirety of reads for each patient specimen paired with a quality metric, for example, in a range from 0 to 64 where a 64 is the best quality and a 0 is the worst quality. In embodiments where both a liquid biopsy sample and a normal tissue sample are sequenced, sequence reads in the corresponding FASTQ files may be matched, such that a liquid biopsy-normal analysis may be performed.

FASTQ format is a text-based format for storing both a biological sequence, such as nucleotide sequence, and its corresponding quality scores. These FASTQ files are analyzed to determine what genetic variants or copy number changes are present in the sample. Each FASTQ file contains reads that may be paired-end or single reads, and may be short-reads or long-reads, where each read represents one detected sequence of nucleotides in a nucleic acid molecule that was isolated from the patient sample or a copy of the nucleic acid molecule, detected by the sequencer. Each read in the FASTQ file is also associated with a quality rating. The quality rating may reflect the likelihood that an error occurred during the sequencing procedure that affected the associated read. In some embodiments, the results of paired-end sequencing of each isolated nucleic acid sample are contained in a split pair of FASTQ files, for efficiency. Thus, in some embodiments, forward (Read 1) and reverse (Read 2) sequences of each isolated nucleic acid sample are stored separately but in the same order and under the same identifier.

RNA Profiling. RNA Profiling May be Achieved, for Instance, Using the Following Methods.

RNA extraction. Transcriptome analysis, the study of the complete set of RNA transcripts that are produced by a cell (the transcriptome), offers a promising means to identify genetic variants that are correlated with disease state and disease progression. For example, to identify genetic variants that are associated with cancer, transcriptome analysis may be performed on a sample collected from a patient that contains cancer cells. Suitable patient samples include tissue samples, tumors (for example, a solid tumor), biopsies, and bodily fluids (for example, blood, serum, plasma, sputum, lavage fluid, cerebrospinal fluid, urine, semen, sweat, tears, saliva). Alternatively, transcriptome analysis may be performed on an organoid that was generated from a human cancer specimen (a “tumor organoid”). Sequencing may be performed on a single cell specimen or on a multi-cell specimen. While RNA sequencing (RNA-seq) can be performed on any patient sample that contains RNA, those of skill in the art will appreciate that the sequencing protocol should be tailored to the particular sample in use. For instance, RNA tends to be highly degraded in tissue samples that have been processed for histology (for example, formalin fixed, paraffin embedded (FFPE) tissue sections). Accordingly, investigators will modify several key steps in the RNA-seq protocol to mitigate sequencing artifacts (see, for example, BMC Medical Genomics 12, 195 (2019)). Today, transcriptome analysis is predominantly performed using high-throughput RNA sequencing (RNA-Seq), which detects the RNA transcripts in a sample using a next-generation sequencer. The first step in performing RNA-seq is to extract RNA from the sample.

Cell Lysis. The first step in extracting RNA from a sample is often to lyse the cells present in that sample. Several physical disruption methods are commonly used to lyse cells, including, for example, mechanical disruption (for example, using a blender or tissue homogenizer), liquid homogenization (for example, using a dounce or French press), high frequency sound waves (for example, using a sonicator), freeze/thaw cycles, heating, manual grinding (for example, using a mortar and pestle), and bead-beating (for example, using a Mini-beadbeater-96 from BioSpec). Cells are also commonly lysed using reagents that contain a detergent, many of which are commercially available (for example, QIAzol Lysis Reagent from QIAGEN, FastBreak™ Cell Lysis Reagent from Promega). Often, physical disruption methods are performed in a “homogenization buffer” that contains, for example, lysis reagents such as detergents or proteases (for example, proteinase K) that increase the efficiency of lysis. Homogenization buffers may also include anti-foaming agents and/or RNase inhibitors to protect RNA from degradation. Those of skill in the art will appreciate that different cell lysis techniques may be required to obtain the best possible yield from different tissues. Techniques that minimize the degradation of the released RNA and that avoid the release of nuclear chromatin are preferred.

RNA isolation. After the cells have been lysed, RNA can be separated from other cellular components. Total RNA is commonly isolated using guanidinium thiocyanate-phenol-chloroform extraction (for example, using TRIzol) or by performing trichloroacetic acid/acetone precipitation followed by phenol extraction. However, there are also many commercially available column-based systems for extracting RNA (for example, PureLink RNA Mini Kit by Invitrogen and Direct-zol Miniprep kit by Zymo Research). Ideally, the isolated RNA will contain very little DNA and enzymatic contamination. To this end, the isolation method may utilize agents that eliminate DNA (for example, TURBO DNase-I), and/or remove enzymatic proteins from the sample (for example, Agencourt® RNAClean® XP beads from Beckman Coulter). In some cases, whole transcriptome sequencing is used to analyze all of the transcripts present in a cell, including messenger RNA (mRNA) as well as all non-coding RNAs. By looking at the whole transcriptome, researchers are able to map exons and introns and to identify splicing variants. Notably, most whole transcription library preparation protocols include a step to remove ribosomal RNA (rRNA), which would otherwise take up the majority of the sequencing reads. Depletion of rRNA is commonly accomplished using a kit, for example, Ribo-Zero Plus rRNA Depletion Kit from Illumina and Seq RiboFree Total RNA Library Kit from Zymo. In other cases, a more targeted RNA-Seq protocol is used to look at a specific type of RNA. For example, mRNA-seq is commonly used to selectively study the “coding” part of the genome, which accounts for only 1-2% of the entire transcriptome. Enriching a sample for mRNA increases the sequencing depth achieved for coding genes, enabling identification of rare transcripts and variants. Polyadenylated mRNAs are commonly enriched for using oligo dT beads (for example, Dynabeads™ from Invitrogen). This enrichment step can be performed either on isolated total RNA or on crude cellular lysate. Targeted approaches have also been developed for the analysis of microRNAs (miRNAs) and small interfering RNAs (siRNAs). These RNAs are commonly isolated using kits that have been designed to efficiently recover small RNAs (for example, mirVana™ miRNA Isolation Kit from Invitrogen).

Library preparation. After RNA has been extracted from the sample, the next major step is to convert the RNA into a form that is suitable for next-generation sequencing (NGS). Through a series of steps, the RNA is converted into a collection of DNA fragments known as a “sequencing library.” After the library has been sequenced, the resulting sequencing “reads” are aligned to a reference genome or transcriptome to determine the expression profile of the analyzed cells. In some cases, library preparation is automated to enable higher sample throughput, minimize errors, and reduce hands-on time. Fully automated library preparation can be performed, for example, using a liquid handling robot (for example, SciClone® NGSx from PerkinElmer).

Reverse transcription. For sequencing, RNA is converted to more stable, double-stranded complementary DNA (cDNA) using reverse transcription (RT). In some cases reverse transcription is performed directly on a sample lysate, prior to RNA isolation. In other cases, reverse transcription is performed on isolated RNA. Reverse transcription is catalyzed by reverse transcriptase, an enzyme that uses an RNA template and a short primer complementary to the 3′ end of the RNA to synthesize a complementary strand of cDNA. This first strand of cDNA is then made double-stranded, either by subjecting it to PCR or using a combination of DNA Polymerase I and DNA Ligase. In the latter method, an RNase (for example, RNase H) is commonly used to digest the RNA strand, allowing the first cDNA strand to serve as a template for synthesis of the second cDNA strand. Many reverse transcriptases are commercially available, including Avian Myeloblastosis Virus (AMV) reverse transcriptases (for example, AMV Reverse Transcriptase from New England BioLabs) and Moloney Murine Leukemia Virus (M-MuLV, MMLV) reverse transcriptases (for example, SMARTscribe™ from Clontech, SuperScript II™ from Life Technologies, and Maxima H Minus™ from Thermo Scientific). Notably, many of the available reverse transcriptases have been engineered for improved thermostability or efficiency (for example, by eliminating 3′→5′ exonuclease activity or reducing RNase H activity).

The primers, which serve as a starting point for synthesis of the new strand, may be random primers (for example, for RT of any RNA), oligo dT primers (for example, for RT of mRNA), or gene-specific primers (for example, for RT of specific target RNAs). Following reverse transcription, an exonuclease (for example, Exonuclease I) may be added to the samples to degrade any primers that remain from the reaction, preventing them from interfering in subsequent amplification steps.

Fragmentation and size selection. Because most sequencing technologies cannot readily analyze long DNA strands, DNA is commonly fragmented into uniform pieces prior to sequencing. The optimal fragment length depends on both the sample type and the sequencing platform to be used. For example, whole genome sequencing typically works best with fragments of DNA that are ˜350 bp long, while targeted sequencing using hybridization capture works best with fragments of DNA that are ˜200 bp long. In some cases, fragmentation is performed after reverse transcription (for example, on cDNA). Suitable methods for fragmenting DNA include physical methods (for example, using sonication, acoustics, nebulization, centrifugal force, needles, or hydrodynamics), enzymatic methods (for example, using NEBNext dsDNA Fragmentase from New England BioLabs), and tagmentation (for example, using the Nextera™ system from Illumina). In other cases, fragmentation is performed prior to reverse transcription (for example, on RNA). In addition to the fragmentation methods that are suitable to DNA, RNA may also be fragmented using heat and magnesium (for example, using the KAPA Hyper Prep Kit from Roche).

A size selection step may subsequently be performed to enrich the library for fragments of an optimal length or range of lengths. Traditionally, size selection was accomplished by separating differentially sized fragments using agarose gel electrophoresis, cutting out the fragments of the desired sizes, and performing a gel extraction (for example, using a MinElute Gel Extraction Kit™ from Qiagen). However, size selection is now commonly accomplished using magnetic bead-based systems (for example, AMPure XP™ from Beckman Coulter, ProNex® Size-Selective Purification System from Promega).

Adapter ligation. Prior to sequencing, the cDNA fragments are ligated to sequencing adapters. Sequencing adapters are short DNA oligonucleotides that contain (1) sequences needed to amplify the cDNA fragment during the sequencing reaction, and (2) sequences that interact with the NGS platform (for example, the surface of the Illumina flow-cell or Ion Torrent beads). Accordingly, adapters must be selected based on the sequencing platform that is to be used.

Libraries from multiple samples are commonly pooled and analyzed in a single sequencing run. To track the source of each cDNA in a pooled sample, a unique molecular barcode (or combination of multiple barcodes) is included in the adapters that are ligated to the cDNA fragments in each library. During the sequencing reaction, the sequencer reads this barcode sequence in addition to the cDNA's biological base sequence. The barcodes are then used to assign each cDNA to its sample of origin during data analysis, a process termed “demultiplexing”. The indexing strategy used for a sequencing reaction should be selected based on the number of pooled samples and the level of accuracy desired. For example, unique dual indexing, in which unique identifiers are added to both ends of the cDNA fragments, is commonly used to ensure that libraries will demultiplex with high accuracy. Adapters may also include unique molecular identifiers (UMIs), short sequences, often with degenerate bases, that incorporate a unique barcode onto each molecule within a given sample library. UMIs reduce the rate of false-positive variant calls and increase sensitivity of variant detection by allowing true variants to be distinguished from errors introduced during library preparation, target enrichment, or sequencing. Many index sequences and adapter sets are commercially available including, for example, SeqCap Dual End Adapters from Roche, xGen Dual Index UMI Adapters from IDT, and TruSeq UD Indexes from Illumina.

Amplification. While it may not be required for some sequencing applications, library preparation typically includes at least one amplification step to enrich for sequencing-competent DNA fragments (for example, fragments with adapter ligated ends) and to generate a sufficient amount of library material for downstream processing. Amplification may be performed using a standard polymerase chain reaction (PCR) technique. However, when possible, care should be taken to minimize amplification bias and limit the introduction of sequencing artifacts. This is accomplished through selection of an appropriate enzyme and protocol parameters. To this end, several companies offer high-fidelity DNA polymerases (for example, KAPA HiFi DNA Polymerase from Roche), which have been shown to produce more accurate sequencing data. Often these DNA polymerases are purchased as part of a PCR master mix (for example, NEBNext® High-Fidelity 2×PCR Master Mix from New England BioLabs) or as part of a kit (for example, KAPA HiFi Library Amplification kit by Roche). Those of skill in the art will appreciate that PCR conditions must be fine-tuned for each sequencing experiment, even when a highly-optimized PCR protocol is used. For example, depending on the initial concentration of DNA in the library and on the input requirement of the sequencer to be used, it may be desirable to subject the library to anywhere from 4-14 cycles of PCR. In some cases, library preparation protocols include multiple rounds of library amplification. For example, in some cases, an additional round of amplification followed by PCR clean-up is performed after the libraries have been pooled.

Clean-up. Following PCR, the amplified DNA is typically purified to remove enzymes, nucleotides, primers, and buffer components that remain from the reaction. Purification is commonly accomplished using phenol-chloroform extraction followed by ethanol precipitation or using a spin column that contains a silica matrix to which DNA selectively binds in the presence of chaotropic salts. Many column-based PCR cleanup kits are commercially available including, for example, those from Qiagen (for example, MinElute PCR Purification Kit), Zymo Research™ (DNA Clean & Concentrator™-5), and Invitrogen (for example, PureLink™ PCR Purification Kit). Alternatively, purification may be accomplished using paramagnetic beads (for example, Axygen™ AxyPrep Mag™ PCR Clean-up Kit).

Pooling. To keep sequencing cost-effective, researchers often pool together multiple libraries, each with a unique barcode, to be sequenced in a single run. The sequencer to be used and the desired sequencing depth should dictate the number of samples that are pooled. For example, for some applications it is advantageous to pool fewer than 12 libraries to achieve greater sequencing depth, whereas for other applications it may be advisable to pool more than 100 libraries. Importantly, if multiple libraries are sequenced in a single run, care should be taken to ensure that the sequencing coverage is roughly equal for each library. To this end, an equal amount of each library (based on molarity) should be pooled. Further, the total molarity of the pooled libraries must be compatible with the sequencer. Thus, it is important to accurately quantify the DNA in the libraries (for example, using the methods discussed in herein) and to perform the necessary calculations before pooling the libraries. In some cases, to achieve a suitable total molarity, it may be necessary to concentrate the pooled libraries, for example, using a vacufuge.

Enrichment. For some applications, it is not necessary to sequence the entire transcriptome of a sample. Instead, “targeted sequencing” may be used to study a select set of genes or specific genomic elements. Libraries that are enriched for target sequences are commonly prepared using hybridization based methods (for example, hybridization capture-based target enrichment). Hybridization may be performed either on a solid surface (microarray) or in solution. In the solution based method, a pool of biotinylated oligonucleotide probes that specifically hybridize with the genes or genomic elements of interest is added to the library. The probes are then captured and purified using streptavidin-coated magnetic beads, and the sequences that hybridized to these probes are subsequently amplified and sequenced. Many probe panels for library enrichment are commercially available, including those from IDT (for example, xGen Exome Research Panel v1.0 probes) and Roche (for example, SeqCap® probes). Importantly, many available probe panels can be customized, allowing investigators to design sets of capture probes that are precisely tailored to a particular application. In addition, many kits (for example, SeqCap EZ MedExome Target Enrichment Kit from Roche) and hybridization mixes (for example, xGen Lockdown from IDT) that facilitate target enrichment are available for purchase. In some cases, it may be advantageous to treat the libraries with reagents that reduce off-target capture prior to performing target enrichment. For example, libraries are commonly treated with oligonucleotides that bind to adapter sequences (for example, xGen Blocking Oligos) or to repetitive sequences (for example, human Cot DNA) to reduce non-specific binding to the capture probes.

In one example, a sequencer may generate a BCL file. A BCL file may include raw image data of a plurality of patient specimens which are sequenced. BCL image data is an image of the flow cell across each cycle during sequencing. A cycle may be implemented by illuminating a patient specimen with a specific wavelength of electromagnetic radiation, generating a plurality of images which may be processed into base calls via BCL to FASTQ processing algorithms which identify which base pairs are present at each cycle. The resulting FASTQ may then comprise the entirety of reads for each patient specimen paired with a quality metric in a range from 0 to 64 where a 64 is the best quality and a 0 is the worst quality. A patient's tumor specimen and a patient's normal specimen may be matched after sequencing such that a tumor-normal analysis may be performed.

Each FASTQ file contains reads that may be paired-end or single reads, and may be short-reads or long-reads, where each read represents one detected sequence of nucleotides in a DNA molecule that was isolated from the patient sample, a copy of the DNA molecule or a cDNA molecule or copy of the cDNA molecule, where the cDNA molecule was derived from an RNA molecule isolated from the patient sample, detected by the sequencer. Each read in the FASTQ file is also associated with a quality rating. The quality rating may reflect the likelihood that an error occurred during the sequencing procedure that affected the associated read.

Differential gene expression analysis. One use of RNA-seq data is to identify genes that are differentially expressed between two or more experimental groups. For example, RNA sequencing data can be used to identify genes that are expressed at significantly higher or lower levels in patients (for example, patients having cancer, autoimmune disease(s), an infection, and/or transplantation requirement) as compared to healthy individuals. This may be accomplished by performing a statistical analysis to compare the normalized read count of each gene across the different experimental groups. The aim of this analysis is to determine whether any observed difference in read count is significant, i.e., whether it is greater than what would be expected just due to natural random variation.

Several data processing steps may be performed to prepare the raw sequencing data for analysis. Sequencing data is typically supplied in FASTQ format, in which each sequencing read is associated with a quality score. First, the data is processed to remove sequencing artifacts, e.g., adaptor sequences and low-complexity reads. Sequencing errors are identified based on the read quality score and are removed or corrected. Publicly available tools, such as Tag Dust, SeqTrim, and Quake, can be used to perform these “data grooming” steps.

During the next stage of data processing, the reads are aligned to a reference genome using an alignment tool. Several publicly available tools can be used for this step including, for example, Kallisto or other pseudo alignment tools, or alignment tools including TopHat, Cufflinks, and Scripture. These programs can be used to reconstruct transcripts, identify variants, and quantitate expression levels for each transcript and gene.

After the reads have been aligned and quantitated, a differential expression analysis may be performed. Statistical methods that are commonly used for differential expression analysis include those based on negative binomial distributions (e.g., edgeR and DESeq) and Bayesian approaches based on a negative binomial model (e.g., baySeq and EBSeq).

Bioinformatics pipeline. In certain aspects, the bioinformatics pipeline includes the systems and methods disclosed in this document. The bioinformatics methods may include filtering NGS reads (for example, according to quality scores or other characteristics associated with each read), aligning reads to a reference genome, detecting fusions having a 5′ partner sequence and a 3′ partner sequence, analyzing read depths or other potentially relevant coverage factors and the status of a partner sequence as in-frame or out-of-frame, labeling fusions, and storing the labeled fusion data in a database. In one example, in-frame means that the number of nucleotides between the final nucleotide of the 3′ partner sequence and the starting nucleotide of the 5′ partner sequence is a number divisible by three. If that condition is not met, the fusion sequence is classified as out-of-frame.

In various embodiments, the bioinformatics pipeline may filter FASTQ data from the corresponding sequence data file for each respective biological sample. Such filtering may include correcting or masking sequencer errors and removing (trimming) low quality sequences or bases, adapter sequences, contaminations, chimeric reads, overrepresented sequences, biases caused by library preparation, amplification, or capture, and other errors.

While workflow 200 illustrates steps for obtaining a biological sample, extracting nucleic acids from the biological sample, and sequencing the isolated nucleic acids, in some embodiments, sequencing data used in the improved systems and methods described herein (for example, which include improved methods for fusion pathogenicity scoring) is obtained by receiving previously generated sequence reads, in electronic form.

Referring again to FIG. 2A, nucleic acid sequencing data 122 generated from the one or more patient samples is then evaluated (for example, via variant analysis 206) in a bioinformatics pipeline, for example, using bioinformatics module 140 of system 100, to identify genomic alterations and other metrics in the cancer genome of the patient. An example overview for a bioinformatics pipeline is described below. Advantageously, in some embodiments, the present disclosure improves bioinformatics pipelines, like pipeline 206, by improving fusion pathogenicity.

FIG. 4A illustrates an example bioinformatics pipeline 206 (for example, as used for feature extraction in the workflows illustrated in FIGS. 2A and 3) for providing clinical support for precision oncology. As shown in FIG. 4A, sequencing data 122 obtained from the wet lab processing 204 (for example, sequence reads 314) is input into the pipeline.

In some embodiments, the sequencing data is processed (for example, using sequence data processing module 141) to prepare it for genomic feature identification 385. For instance, in some embodiments as described above, the sequencing data is present in a native file format provided by the sequencer. Accordingly, in some embodiments, the system (for example, system 100) applies a pre-processing algorithm 142 to convert the file format (318) to one that is recognized by one or more upstream processing algorithms. For example, BCL file outputs from a sequencer can be converted to a FASTQ file format using the bcl2fastq or bcl2fastq2 conversion software (Illumina®). FASTQ format is a text-based format for storing both a biological sequence, such as nucleotide sequence, and its corresponding quality scores. These FASTQ files are analyzed to determine what genetic variants, copy number changes, etc., are present in the sample.

FASTQ and filtering. In some embodiments, other preprocessing steps are performed, for example, filtering sequence reads 122 based on a desired quality, for example, size and/or quality of the base calling. In some embodiments, quality control checks are performed to ensure the data is sufficient for variant calling. For instance, entire reads, individual nucleotides, or multiple nucleotides that are likely to have errors may be discarded based on the quality rating associated with the read in the FASTQ file, the known error rate of the sequencer, and/or a comparison between each nucleotide in the read and one or more nucleotides in other reads that has been aligned to the same location in the reference genome. Filtering may be done in part or in its entirety by various software tools, for example, a software tool such as Skewer. See, Jiang, H. et al., BMC Bioinformatics 15(182):1-12 (2014). FASTQ files may be analyzed for rapid assessment of quality control and reads, for example, by a sequencing data QC software such as AfterQC, Kraken, RNA-SeQC, FastQC, or another similar software program. For paired-end reads, reads may be merged.

In some embodiments, when both a liquid biopsy sample and a normal tissue sample from the patient are sequenced, two FASTQ output files are generated, one for the liquid biopsy sample and one for the normal tissue sample. A ‘matched’ (for example, panel-specific) workflow is run to jointly analyze the liquid biopsy-normal matched FASTQ files. When a matched normal sample is not available from the patient, FASTQ files from the liquid biopsy sample are analyzed in the ‘tumor-only’ mode. See, for example, FIG. 4B. If two or more patient samples are processed simultaneously on the same sequencer flow cell, for example, a liquid biopsy sample and a normal tissue sample, a difference in the sequence of the adapters used for each patient sample barcodes nucleic acids extracted from both samples, to associating each read with the correct patient sample and facilitate assignment to the correct FASTQ file.

For efficiency, in some embodiments, the results of paired-end sequencing of each isolate are contained in a split pair of FASTQ files. Forward (Read 1) and reverse (Read 2) sequences of each tumor and normal isolate are stored separately but in the same order and under the same identifier. In various embodiments, the bioinformatics pipeline may filter FASTQ data from each isolate. Such filtering may include correcting or masking sequencer errors and removing (trimming) low quality sequences or bases, adapter sequences, contaminations, chimeric reads, overrepresented sequences, biases caused by library preparation, amplification, or capture, and other errors.

Similarly, in some embodiments, sequencing (312) is performed on a pool of nucleic acid sequencing libraries prepared from different biological samples, for example, from the same or different patients. Accordingly, in some embodiments, the system demultiplexes (320) the data (for example, using demultiplexing algorithm 144) to separate sequence reads into separate files for each sequencing library included in the sequencing pool, for example, based on UMI or patient identifier sequences added to the nucleic acid fragments during sequencing library preparation, as described above. In some embodiments, the demultiplexing algorithm is part of the same software package as one or more pre-processing algorithms 142. For instance, the bcl2fastq or bcl2fastq2 conversion software (Illumina®) include instructions for both converting the native file format output from the sequencer and demultiplexing sequence reads 122 output from the reaction.

FASTQ and alignment. The sequence reads are then aligned (322), for example, using an alignment algorithm 143, to a reference sequence construct 158, for example, a reference genome, reference exome, or other reference construct prepared for a particular targeted-panel sequencing reaction. For example, in some embodiments, individual sequence reads 123, in electronic form (for example, in FASTQ files), are aligned against a reference sequence construct for the species of the subject (for example, a reference human genome) by identifying a sequence in a region of the reference sequence construct that best matches the sequence of nucleotides in the sequence read. In some embodiments, the sequence reads are aligned to a reference exome or reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. Any of a variety of alignment tools can be used for this task.

For instance, local sequence alignment algorithms compare subsequences of different lengths in the query sequence (for example, sequence read) to subsequences in the subject sequence (for example, reference construct) to create the best alignment for each portion of the query sequence. In contrast, global sequence alignment algorithms align the entirety of the sequences, for example, end to end. Examples of local sequence alignment algorithms include the Smith-Waterman algorithm (see, for example, Smith and Waterman, J Mol. Biol., 147(1):195-97 (1981), which is incorporated herein by reference), Lalign (see, for example, Huang and Miller, Adv. Appl. Math, 12:337-57 (1991), which is incorporated by reference herein), and PatternHunter (see, for example, Ma B. et al., Bioinformatics, 18(3):440-45 (2002), which is incorporated by reference herein).

In some embodiments, the read mapping process starts by building an index of either the reference genome or the reads, which is then used to retrieve the set of positions in the reference sequence where the reads are more likely to align. Once this subset of possible mapping locations has been identified, alignment is performed in these candidate regions with slower and more sensitive algorithms. See, for example, Hatem et al., 2013, “Benchmarking short sequence mapping tools,” BMC Bioinformatics 14: p. 184; and Flicek and Birney, 2009, “Sense from sequence reads: methods for alignment and assembly,” Nat Methods 6(Suppl. 11), S6-S12, each of which is hereby incorporated by reference. In some embodiments, the mapping tools methodology makes use of a hash table or a Burrows-Wheeler transform (BWT). See, for example, Li and Homer, 2010, “A survey of sequence alignment algorithms for next-generation sequencing,” Brief Bioinformatics 11, pp. 473-483, which is hereby incorporated by reference.

Other software programs designed to align reads include, for example, Novoalign (Novocraft, Inc.), Bowtie, Burrows Wheeler Aligner (BWA), and/or programs that use a Smith-Waterman algorithm. Candidate reference genomes include, for example, hg19, GRCh38, hg38, GRCh37, and/or other reference genomes developed by the Genome Reference Consortium. In some embodiments, the alignment generates a SAM file, which stores the locations of the start and end of each read according to coordinates in the reference genome and the coverage (number of reads) for each nucleotide in the reference genome.

For example, in some embodiments, each read of a FASTQ file is aligned to a location in the human genome having a sequence that best matches the sequence of nucleotides in the read. There are many software programs designed to align reads, for example, Novoalign (Novocraft, Inc.), Bowtie, Burrows Wheeler Aligner (BWA), programs that use a Smith-Waterman algorithm, etc. Alignment may be directed using a reference genome (for example, hg19, GRCh38, hg38, GRCh37, other reference genomes developed by the Genome Reference Consortium, etc.) by comparing the nucleotide sequences in each read with portions of the nucleotide sequence in the reference genome to determine the portion of the reference genome sequence that is most likely to correspond to the sequence in the read. In some embodiments, one or more SAM files are generated for the alignment, which store the locations of the start and end of each read according to coordinates in the reference genome and the coverage (number of reads) for each nucleotide in the reference genome. The SAM files may be converted to BAM files. In some embodiments, the BAM files are sorted and duplicate reads are marked for deletion, resulting in de-duplicated BAM files.

This process produces a tumor BAM file, and a normal BAM file (when available). In some embodiments, where both a liquid biopsy sample and a normal tissue sample are analyzed, this process produces a liquid biopsy BAM file (for example, Liquid BAM 124-1-i-cf) and a normal BAM file (for example, Germline BAM 124-1-i-g), as illustrated in FIG. 4A. In various embodiments, BAM files may be analyzed to detect genetic variants and other genetic features, including single nucleotide variants (SNVs), copy number variants (CNVs), gene rearrangements, etc.

In some embodiments, the sequencing data is normalized, for example, to account for pull-down, amplification, and/or sequencing bias (for example, mappability, GC bias etc.). See, for example, Schwartz et al., PLoS ONE 6(1):e16685 (2011) and Benjamini and Speed, Nucleic Acids Research 40(10):e72 (2012), the contents of which are hereby incorporated by reference, in their entireties, for all purposes.

In some embodiments, SAM files generated after alignment are converted to BAM files 124. Thus, after preprocessing sequencing data generated for a pooled sequencing reaction, BAM files are generated for each of the sequencing libraries present in the master sequencing pools. For example, as illustrated in FIG. 4A, separate BAM files are generated for each of three samples acquired from subject 1 at time i (for example, tumor BAM 124-1-i-t corresponding to alignments of sequence reads of nucleic acids isolated from a solid tumor sample from subject 1, Liquid BAM 124-1-i-cf corresponding to alignments of sequence reads of nucleic acids isolated from a liquid biopsy sample from subject 1, and Germline BAM 124-1-i-g corresponding to alignments of sequence reads of nucleic acids isolated from a normal tissue sample from subject 1), and one or more samples acquired from one or more additional subjects at time j (for example, Tumor BAM 124-2-j-t corresponding to alignments of sequence reads of nucleic acids isolated from a solid tumor sample from subject 2). In some embodiments, BAM files are sorted, and duplicate reads are marked for deletion, resulting in de-duplicated BAM files. For example, tools like SamBAMBA mark and filter duplicate alignments in the sorted BAM files.

Many of the embodiments described below, in conjunction with FIG. 4A, relate to analyses performed using sequencing data from cfDNA of a cancer patient, for example, obtained from a liquid biopsy sample of the patient. Generally, these embodiments are independent and, thus, not reliant upon any particular sequencing data generation methods, for example, sample preparation, sequencing, and/or data pre-processing methodologies. However, in some embodiments, the methods described below include one or more steps 204 of generating sequencing data, as illustrated in FIGS. 2A and 3.

Alignment files prepared as described above (for example, BAM files 124) are then passed to a feature extraction module 145, where the sequences are analyzed (324) to identify genomic alterations (for example, SNVs/MNVs, indels, genomic rearrangements, copy number variations, etc.) and/or determine various characteristics of the patient's cancer (for example, MSI status, TMB, tumor ploidy, HRD status, tumor fraction, tumor purity, methylation patterns, etc.). Many software packages for identifying genomic alterations are known in the art, for example, freebayes, PolyBayse, samtools, GATK, pindel, SAMtools, Breakdancer, Cortex, Crest, Delly, Gridss, Hydra, Lumpy, Manta, and Socrates. For a review of many of these variant calling packages see, for example, Cameron, D. L. et al., Nat. Commun., 10(3240):1-11 (2019), the content of which is hereby incorporated by reference, in its entirety, for all purposes. Generally, these software packages identify variants in sorted SAM or BAM files 124, relative to one or more reference sequence constructs 158. The software packages then output a file for example, a raw VCF (variant call format), listing the variants (for example, genomic features 131) called and identifying their location relevant to the reference sequence construct (for example, where the sequence of the sample nucleic acids differ from the corresponding sequence in the reference construct). In some embodiments, system 100 digests the contents of the native output file to populate feature data 125 in test patient data store 120. In other embodiments, the native output file serves as the record of these genomic features 131 in test patient data store 120.

Generally, the systems described herein can employ any combination of available variant calling software packages and internally developed variant identification algorithm. In some embodiments, the output of a particular algorithm of a variant calling software is further evaluated, for example, to improve variant identification. Accordingly, in some embodiments, system 100 employs an available variant calling software package to perform some of all of the functionality of one or more of the algorithms shown in feature extraction module 145.

In some embodiments, as illustrated in FIG. 1A, separate algorithms (or the same algorithm implemented using different parameters) are applied to identify variants unique to the cancer genome of the patient and variants existing in the germline of the subject. In other embodiments, variants are identified indiscriminately and later classified as either germline or somatic, for example, based on sequencing data, population data, or a combination thereof. In some embodiments, variants are classified as germline variants, and/or non-actionable variants, when they are represented in the population above a threshold level, for example, as determined using a population database such as ExAC or gnomAD. For instance, in some embodiments, variants that are represented in at least 1% of the alleles in a population are annotated as germline and/or non-actionable. In other embodiments, variants that are represented in at least 2%, at least 3%, at least 4%, at least 5%, at least 7.5%, at least 10%, or more of the alleles in a population are annotated as germline and/or non-actionable. In some embodiments, sequencing data from a matched sample from the patient, for example, a normal tissue sample, is used to annotate variants identified in a cancerous sample from the subject. That is, variants that are present in both the cancerous sample and the normal sample represent those variants that were in the germline prior to the patient developing cancer, and can be annotated as germline variants.

In various aspects, the detected genetic variants and genetic features are analyzed as a form of quality control. For example, a pattern of detected genetic variants or features may indicate an issue related to the sample, sequencing procedure, and/or bioinformatics pipeline (for example, example, contamination of the sample, mislabeling of the sample, a change in reagents, a change in the sequencing procedure and/or bioinformatics pipeline, etc.).

This particular workflow is only an example of one possible collection and arrangement of algorithms for feature extraction from sequencing data 124. Generally, any combination of the modules and algorithms of feature extraction module 145, for example, illustrated in FIG. 1A, can be used for a bioinformatics pipeline. For instance, in some embodiments, an architecture useful for the methods and systems described herein includes at least one of the modules or variant calling algorithms shown in feature extraction module 145. In some embodiments, an architecture includes at least 2, 3, 4, 5, 6, 7, 8, 9, 10, or more of the modules or variant calling algorithms shown in feature extraction module 145. Further, in some embodiments, feature extraction modules and/or algorithms not illustrated in FIG. 1A find use in the methods and systems described herein.

Variant Detection. In some embodiments, variant analysis of aligned sequence reads, for example, in SAM or BAM format, includes identification of single nucleotide variants (SNVs), multiple nucleotide variants (MNVs), indels (for example, nucleotide additions and deletions), and/or genomic rearrangements (for example, inversions, translocations, and gene fusions) using variant identification module 146, for example, which includes a SNV/MNV calling algorithm (for example, SNV/MNV calling algorithm 147), an indel calling algorithm (for example, indel calling algorithm 148), and/or one or more genomic rearrangement calling algorithms (for example, genomic rearrangement calling algorithm 149). Essentially, the module first identifies a difference between the sequence of an aligned sequence read 124 and the reference sequence to which the sequence read is aligned (for example, an SNV/MNV, an indel, or a genomic rearrangement) and makes a record of the variant, for example, in a variant call format (VCF) file. For instance, software packages such as freebayes and pindel are used to call variants using sorted BAM files and reference BED files as the input. For a review of variant calling packages see, for example, Cameron, D. L. et al., Nat. Commun., 10(3240):1-11 (2019). A raw VCF file (variant call format) file is output, showing the locations where the nucleotide base in the sample is not the same as the nucleotide base in that position in the reference sequence construct.

In some embodiments, raw VCF data is then normalized, for example, by parsimony and left alignment. For example, software packages such as vcfbreakmulti and vt are used to normalize multi-nucleotide polymorphic variants in the raw VCF file and a variant normalized VCF file is output. See, for example, E. Garrison, “Vcflib: A C++ library for parsing and manipulating VCF files, GitHub https://github.com/ekg/vcflib (2012), the content of which is hereby incorporated by reference, in its entirety, for all purposes. In some embodiments, a normalization algorithm is included within the architecture of a broader variant identification software package.

An algorithm is then used to annotate the variants in the (for example, normalized) VCF file, for example, determines the source of the variation, for example, whether the variant is from the germline of the subject (for example, a germline variant), a cancerous tissue (for example, a somatic variant), a sequencing error, or of an undeterminable source. In some embodiments, an annotation algorithm is included within the architecture of a broader variant identification software package. However, in some embodiments, an external annotation algorithm is applied to (for example, normalized) VCF data obtained from a conventional variant identification software package. The choice to use a particular annotation algorithm is well within the purview of the skilled artisan, and in some embodiments is based upon the data being annotated.

For example, in some embodiments, where both a liquid biopsy sample and a normal tissue sample of the patient are analyzed, variants identified in the normal tissue sample inform annotation of the variants in the liquid biopsy sample. In some embodiments, where a particular variant is identified in the normal tissue sample, that variant is annotated as a germline variant in the liquid biopsy sample. Similarly, in some embodiments, where a particular variant identified in the liquid biopsy sample is not identified in the normal tissue sample, the variant is annotated as a somatic variant when the variant otherwise satisfies any additional criteria placed on somatic variant calling, for example, a threshold variant allele frequency (VAF) in the sample.

By contrast, in some embodiments, where only a liquid biopsy sample is being analyzed, the annotation algorithm relies on other characteristics of the variant in order to annotate the origin of the variant. For instance, in some embodiments, the annotation algorithm evaluates the VAF of the variant in the sample, for example, alone or in combination with additional characteristics of the sample, for example, tumor fraction. Accordingly, in some embodiments, where the VAF is within a first range encompassing a value that corresponds to a 1:1 distribution of variant and reference alleles in the sample, the algorithm annotates the variant as a germline variant, because it is presumably represented in cfDNA originating from both normal and cancer tissues. Similarly, in some embodiments, where the VAF is below a baseline variant threshold, the algorithm annotates the variant as undeterminable, because there is not sufficient evidence to distinguish between the possibility that the variant arose as a result of an amplification or sequencing error and the possibility that the variant originated from a cancerous tissue. Similarly, in some embodiments, where the VAF falls between the first range and the baseline variant threshold, the algorithm annotates the variant as a somatic variant.

In some embodiments, the baseline variant threshold is a value from 0.01% VAF to 0.5% VAF. In some embodiments, the baseline variant threshold is a value from 0.05% VAF to 0.35% VAF. In some embodiments, the baseline variant threshold is a value from 0.1% VAF to 0.25% VAF. In some embodiments, the baseline variant threshold is about 0.01% VAF, 0.015% VAF, 0.02% VAF, 0.025% VAF, 0.03% VAF, 0.035% VAF, 0.04% VAF, 0.045% VAF, 0.05% VAF, 0.06% VAF, 0.07% VAF, 0.075% VAF, 0.08% VAF, 0.09% VAF, 0.1% VAF, 0.15% VAF, 0.2% VAF, 0.25% VAF, 0.3% VAF, 0.35% VAF, 0.4% VAF, 0.45% VAF, 0.5% VAF, or greater. In some embodiments, the baseline variant threshold is different for variants located in a first region, for example, a region identified as a mutational hotspot and/or having high genomic complexity, than for variants located in a second region, for example, a region that is not identified as a mutational hotspot and/or having average genomic complexity. For example, in some embodiments, the baseline variant threshold is a value from 0.01% to 0.25% for variants located in the first region and is a value from 0.1% to 0.5% for variants located in the second region. In some embodiments, a baseline variant threshold is influenced by the sequencing depth of the reaction, for example, a locus-specific sequencing depth and/or an average sequencing depth (for example, across a targeted panel and/or complete reference sequence construct). In some embodiments, the baseline variant threshold is dependent upon the type of variant being detected. For example, in some embodiments, different baseline variant thresholds are set for SNPs/MNVs than for indels and/or genomic rearrangements. For instance, while an apparent SNP may be introduced by amplification and/or sequencing errors, it is much less likely that a genomic rearrangement is introduced this way and, thus, a lower baseline variant threshold may be appropriate for genomic rearrangements than for SNPs/MNVs.

In some embodiments, one or more additional criteria are required to be satisfied before a variant can be annotated as a somatic variant. For instance, in some embodiments, a threshold number of unique sequence reads encompassing the variant must be present to annotate the variant as somatic. In some embodiments, the threshold number of unique sequence reads is only applied when certain conditions are met, for example, when the variant allele is located in a region of average genomic complexity. In some embodiments, a threshold sequencing coverage, for example, a locus-specific and/or an average sequencing depth (for example, across a targeted panel and/or complete reference sequence construct) must be satisfied to annotate the variant as somatic. In some embodiments, bases contributing to the variant must satisfy a threshold mapping quality to annotate the variant as somatic. In some embodiments, alignments contributing to the variant must satisfy a threshold alignment quality to annotate the variant as somatic. In some embodiments, one or more genomic regions is blacklisted, preventing somatic variant annotation for variants falling within the region. In various embodiments, any combination of the additional criteria, as well as additional criteria not listed above, may be applied to the variant calling process. Again, in some embodiments, different criteria are applied to the annotation of different types of variants.

Fusion Detection. In some embodiments, genomic rearrangements (for example, inversions, translocations, and gene fusions) are detected following de-multiplexing by aligning tumor FASTQ files against a human reference genome using a local alignment algorithm, such as BWA. In some embodiments, DNA reads are sorted and duplicates may be marked with a software, for example, SAMBlaster. Discordant and split reads may be further identified and separated. These data may be read into a software, for example, LUMPY, for structural variant detection, including candidate fusion detection, as part of a fusion detection pipeline. Examples of fusion detection software packages include Pizzly, STAR, MOJO, etc. (see https://github.com/pmelsted/pizzly, https://github.com/STAR-Fusion/STAR-Fusion/wiki, https://github.com/cband/MOJO) In various embodiments, the fusion detection pipeline may be hosted on one or more docker images. In some embodiments, structural alterations are grouped by type, recurrence, and presence and stored within a database and displayed through a fusion viewer software tool. The fusion viewer software tool may reference a database, for example, Ensembl, to determine the gene and proximal exons surrounding the breakpoint for any possible transcript generated across the breakpoint. The fusion viewer tool may then place the breakpoint 5′ or 3′ to the subsequent exon in the direction of transcription. For inversions, this orientation may be reversed for the inverted gene. After positioning of the breakpoint, the translated amino acid sequences may be generated for both genes in the chimeric protein, and a plot may be generated containing the remaining functional domains for each protein, as returned from a database, for example, Uniprot.

For instance, in an example implementation, gene rearrangements are detected using the SpeedSeq analysis pipeline. Chiang et al., 2015, “SpeedSeq: ultra-fast personal genome analysis and interpretation,” Nat Methods, (12), pg. 966. Briefly, FASTQ files are aligned to hg19 using BWA. Split reads mapped to multiple positions and read pairs mapped to discordant positions are identified and separated, then utilized to detect gene rearrangements by LUMPY. Layer et al., 2014, “I. M. LUMPY: a probabilistic framework for structural variant discovery,” Genome Biol, (15), pg. 84. Fusions can then be filtered according to the number of supporting reads.

In various embodiments, fusion detection includes analyzing aligned reads to detect reads having two portions where the portions align to non-contiguous regions of a reference genome. Fusion detection may further include localizing breakpoints in data based on misalignments, ascribing and quantifying the technical, supporting reads spanning the breakpoint, and estimating where breakpoints are located in the genome. If the number of split reads and discordant reads detected for a given breakpoint exceeds a threshold value, the group of reads associated with each breakpoint are grouped as a fusion candidate.

Report. In some embodiments, the methods described herein include a step of generating a clinical report (for example, a patient report) providing clinical support for personalized cancer therapy, using the information curated from sequencing of a liquid biopsy sample, as described above. In some embodiments, the report is provided to a patient, physician, medical personnel, or researcher in a digital copy (for example, a JSON object, a pdf file, or an image on a website or portal), a hard copy (for example, printed on paper or another tangible medium). A report object, such as a JSON object, can be used for further processing and/or display. For example, information from the report object can be used to prepare a clinical laboratory report for return to an ordering physician. In some embodiments, the report is presented as text, as audio (for example, recorded or streaming), as images, or in another format and/or any combination thereof.

The report includes information related to the specific characteristics of the patient's cancer, for example, cancer type, detected genetic variants, epigenetic abnormalities, associated oncogenic pathogenic infections, and/or pathology abnormalities. The report may include information related to detected gene fusions, especially gene fusions ranked likely to be pathogenic by the systems and methods, and other characteristics of a patient's sample and/or clinical records.

In some embodiments, other characteristics of a patient's sample and/or clinical records are also included in the report. For example, in some embodiments, the clinical report includes information on clinical variants, for example, one or more of copy number variants (for example, for actionable genes CCNE1, CD274(PD-L1), EGFR, ERBB2(HER2), MET, MYC, BRCA1, and/or BRCA2), fusions, translocations, and/or rearrangements (for example, in actionable genes ALK, ROS1, RET, NTRK1, FGFR2, FGFR3, NTRK2 and/or NTRK3), pathogenic single nucleotide polymorphisms, insertion-deletions (for example, somatic/tumor and/or germline/normal), therapy biomarkers, gene expression calls (for example, over- or under-expression of a gene compared to the expression level of that gene in normal tissue), microsatellite instability status, and/or tumor mutational burden.

In some embodiments, identified clinical variants are labeled as “potentially actionable”, “biologically relevant”, “variants of unknown significance (VUSs)”, or “benign”. Potentially actionable alterations are protein-altering variants with an associated therapy based on evidence from the medical literature. Biologically relevant alterations are protein-altering variants that may have functional significance or have been observed in the medical literature but are not associated with a specific therapy. Variants of unknown significance (VUSs) are protein-altering variants exhibiting an unclear effect on function and/or without sufficient evidence to determine their pathogenicity. In some embodiments, benign variants are not reported. In some embodiments, variants are identified through aligning the patient's DNA sequence to the human genome reference sequence version hg19 (GRCh37) or RNA sequence to the human genome reference sequence version GRCh38. In some embodiments, actionable and biologically relevant somatic variants are provided in a clinical summary during report generation.

For instance, in some embodiments, variant classification and reporting is performed, where detected variants are investigated following criteria from known evolutionary models, functional data, clinical data, literature, and other research endeavors, including tumor organoid experiments. In some embodiments, variants are prioritized and classified based on known gene-disease relationships, hotspot regions within genes, internal and external somatic databases, primary literature, and other features of somatic drivers. Variants can be added to a patient (or sample, for example, organoid sample) report based on recommendations from the Association for Molecular Pathology (AMP), American Society of Clinical Oncology (ASCO), or College of American Pathologists (CAP) guidelines. Additional guidelines may be followed (for example, National Comprehensive Cancer Network (NCCN) or Food and Drug Administration (FDA). Briefly, pathogenic variants with therapeutic, diagnostic, or prognostic significance may be prioritized in the report. Non-actionable pathogenic variants may be included as biologically relevant, followed by variants of uncertain significance. Translocations may be reported based on outputs from classifier 300, features of known gene fusions, relevant breakpoints, and biological relevance. Evidence may be curated from public and private databases or research and presented as 1) consensus guidelines 2) clinical research, or 3) case studies, with a link to the supporting literature. Germline alterations may be reported as secondary findings in a subset of genes for consenting patients. These may include genes recommended by the ACMG and additional genes associated with cancer predisposition or drug resistance.

In some embodiments, a clinical report includes information about clinical trials for which the patient is eligible, matched therapies that are specific to the patient's cancer, and/or possible therapeutic adverse effects associated with the specific characteristics of the patient's cancer, for example, the patient's genetic variations, epigenetic abnormalities, associated oncogenic pathogenic infections, and/or pathology abnormalities, or other characteristics of the patient's sample and/or clinical records. For example, in some embodiments, the clinical report includes such patient information and analysis metrics, including cancer type and/or diagnosis, variant allele fraction, patient demographic and/or institution, matched therapies (for example, FDA approved and/or investigational), matched clinical trials, variants of unknown significance (VUS), genes with low coverage, panel information, specimen information, details on reported variants, patient clinical history, status and/or availability of previous test results, and/or version of bioinformatics pipeline.

Matched therapies. In exemplary aspects, matched therapies included on the report may be determined based on clinical and scientific guidelines (for example, U.S. Food and Drug Administration or National Cancer Care Network guidelines and/or clinical trial results, etc.) and data associated with the patient and/or specimen, including predicted PD-L1 status 1120 (which may include an indication of the percentage of tumor cells predicted to stain PD-L1 positive, the percentage of a tumor expected to contain PD-L1 positive stained tumor infiltrating lymphocytes, and/or the percentage of tumor infiltrating lymphocytes predicted to stain PD-L1 positive), and characteristics of any data type, including genetic data 1330 (for example, epidermal growth factor receptor/EGFR or anaplastic lymphoma kinase/ALK genomic aberations), cancer type, clinical data 1325 (including patient age, previously prescribed therapies, patient response to first-line therapies, second-line therapies, etc.), and any data relevant to clinical and scientific guidelines (for example, exclusion or inclusion criteria). As clinical and scientific guidelines are updated, matched therapies may change in accordance with changes to guidelines.

In exemplary aspects, the treatment is an immune checkpoint blockade therapy. In various embodiments, immune checkpoint blockade therapy comprises treatment with one or more of ipilimumab, nivolumab, pembrolizumab, atezolizumab, avelumab, durvalumab.

Each therapy may be a monotherapy or a combination of multiple therapies (for example, a combination that includes one or more immune checkpoint blockade therapies or other immunotherapies and may further include one or more non-immunotherapy). In one embodiment, combinations of therapies may include Carboplatin with Paclitaxel; Carboplatin with Pemetrexed; Carboplatin with Paclitaxel and Bevacizumab; Pembrolizumab with Platinum-based chemotherapy (for example, cisplatin or carboplatin) and Pemetrexed; Atezolizumab with Carboplatin, Paclitaxel, and Bevacizumab; Atezolizumab with Carboplatin and Paclitaxel (Protein Bound); Nivolumab with Ipilimumab; Nivolumab with Ipilimumab and platinum-based doublet chemotherapy. In one example, these combinations may be first-line therapies matched for patients having advanced or metastatic non-small cell lung cancer (NSCLC). In other examples, therapies are matched for a variety of cancer types.

In one example, nivolumab with ipilimumab combination therapy may be matched as a first-line therapy, second-line therapy, third-line therapy, etc. for a patient having metastatic NSCLC, no EGFR or ALK genomic tumor aberrations, and a tumor having at least 1% of its cells predicted to stain positive for PD-L1. In another example, nivolumab with ipilimumab and 2 cycles of platinum-doublet chemotherapy combination therapy may be matched as a first-line therapy, second-line therapy, third-line therapy, etc. for a patient having metastatic or recurrent NSCLC and no EGFR or ALK genomic tumor aberrations. In yet another example, atezolizumab may be matched as a first-line therapy, second-line therapy, third-line therapy, etc. for an adult patient having metastatic NSCLC, no EGFR or ALK genomic tumor aberrations, and a tumor having at least 50% of its cells predicted to stain positive for PD-L1 or PD-L1 positive tumor infiltration cells covering at least 10% of the tumor area.

In some other examples, therapies are matched in accordance with U.S. Patent App. Pub. Nos. US 2015/0125463 A1 and/or US 2018/0319892 A1, the contents of which are incorporated herein in their entireties by reference.

In some embodiments, the results included in the report, and/or any additional results (for example, from the bioinformatics pipeline), are used to query a database of clinical data, for example, to determine whether there is a trend showing that a particular therapy was effective or ineffective in treating (for example, slowing or halting cancer progression), and/or adverse effects of such treatments in other patients having the same or similar characteristics.

In some embodiments, the results are used to design cell-based studies of the patient's biology, for example, tumor organoid experiments. For example, an organoid may be genetically engineered to have the same characteristics as the specimen and may be observed after exposure to a therapy to determine whether the therapy can reduce the growth rate of the organoid, and thus may be likely to reduce the growth rate of the cancer in the patient associated with the specimen. Similarly, in some embodiments, the results are used to direct studies on tumor organoids derived directly from the patient. An example of such experimentation is described in U.S. Provisional Patent Application No. 62/944,292, filed Dec. 5, 2019, the content of which is hereby incorporated by reference, in its entirety, for all purposes.

In some embodiments, a clinical report is checked for final validation, review, and sign-off by a medical practitioner (for example, a pathologist). The clinical report is then sent for action (for example, for precision oncology applications).

Human PD-L1, also known as CD274, B7-H, B7H1, PDL1, PD-L1, PDCD1L1, PDCD1LG1, comprises the amino acid sequence of SEQ ID NO: 1 and in some aspects may be encoded by the mRNA sequence SEQ ID NO: 2. These sequences are publicly available at the National Center of Biotechnology Information website as Accession Nos. NP001254635.1 (SEQ ID NO: 1) and NM_001267706.1 (SEQ ID NO: 2). Additional isoforms are known in the art and include the amino acid sequences of SEQ ID NOs: 3 and 5 encoded by SEQ ID NOs: 4 and 6. The gene encoding PD-L1 is located in the human genome at chromosome 9. PD-L1 is known as an immune inhibitory receptor ligand expressed by antigen-presenting cells, macrophages, T cells, and B cells in addition to various types of tumor cells. The interaction of PD-L1 with its receptor, PD-1, leads to inhibition of T cell activation and cytokine production. Thus, it is part of the immune checkpoint pathway and is relevant for preventing autoimmune responses. In the tumor setting, however, interaction of PD-L1 to PD-1 leads to immune escape for the tumor cells. Inhibiting the PD-L1:PD-1 interaction and other checkpoint molecule interactions has become a large focus of cancer research. The development of several therapeutic agents for immune checkpoint blockade therapy has led to the Food and Drug Administration (FDA)-approval of ipilimumab, nivolumab, pembrolizumab, atezolizumab, avelumab, durvalumab, and combinations thereof for the treatment of melanoma, non-small cell lung cancer, renal cell carcinoma, Hodgkin lymphoma, urothelial carcinoma, head and neck squamous cell carcinoma, Merkel cell carcinoma, hepatocellular carcinoma, gastric and gastroesophageal carcinoma, colorectal cancer, and solid tumors (Wei et al., Cancer Discovery (2018); doi: 10.1158/2159-8290.CD-18-0367). In addition to PD-L1, other actors involved in the immune checkpoint pathway include the inhibitory receptors CTLA-4, PD-1, PD-L2, B7-H3, B7-H4, CEACAM-1, TIGIT, LAGS, CD112, CD112R, CD96, TIM3, BTLA, VISTA, and the co-stimulatory receptors ICOS, OX40, 4-1BB, CD27, CD40, and GITR. See, e.g., Wei et al., 2018, supra.

As discussed above, expression of PD-L1 can predict whether immunotherapy treatments, especially immune checkpoint blockade treatments, are likely to successfully eliminate or reduce the number of the patient's cancer cells. While methods of determining PD-L1 status exist, these methods are time-consuming and require relatively large amounts of patient sample (biopsied tissue) which often leads to patient discomfort and inconvenience. In the context of nucleic acid sequencing of patient tumor tissue, additional tissue may not be available for IHC but the sequencing data (RNA sequencing data in particular) represent a source of information that can be used to infer the patient's PD-L1 status. Furthermore, depending on the cancer type, PD-L1 IHC tests are not always ordered, but it may be clinically important to determine whether PD-L1 IHC is a reasonable test to perform as part of clinical decision-making.

Thus, the present disclosure provides a computer-implemented method of identifying programmed-death ligand 1 (PD-L1) expression status of a subject's sample comprising cancer cells. In exemplary aspects, the method comprises (a) receiving an unlabeled expression data set for the subject's sample; (b) aligning the unlabeled expression data set to labeled expression data according to a trained PD-L1 predictive model, wherein the trained PD-L1 predictive model has been trained with a plurality of labeled expression data sets, each labeled expression data set comprising expression data for a sample of a labeled cancer type and a labeled PD-L1 expression status; wherein aligning the unlabeled gene expression data set to labeled expression data according to the trained PD-L1 predictive model identifies PD-L1 expression status for the subject's sample.

As used herein, the term “subject's sample” refers to a biological sample obtained from a subject. In exemplary aspects, the subject's sample comprises a cancer cell. In some embodiments, the sample comprises a bodily fluid, including, but not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of the foregoing samples. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis).

In some embodiments, the subject is a human. In some embodiments, the subject (e.g., human) has cancer.

The cancer referenced herein may be any cancer, e.g., any malignant growth or tumor caused by abnormal and uncontrolled cell division that may spread to other parts of the body through the lymphatic system or the bloodstream. In exemplary aspects, the cancer is one selected from the following cancer types: acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma.

With regard to the presently disclosed computer-implemented method, the plurality of labeled expression data sets comprises expression data for samples of a single cancer type. In this manner, the trained PD-L1 predictive model is considered to be tailored to or specific for one cancer type. In alternative embodiments, the plurality of labeled expression data sets comprises expression data for samples of 2 or more (e.g., 3, 4, 5, 6, 7, 8, 9, 10 or more) cancer types. In such cases, the trained PD-L1 predictive model is considered a “pan-cancer” model. In exemplary aspects, wherein the plurality of labeled expression data sets comprises expression data for breast cancer, prostate cancer, colorectal cancer, lung cancer, skin cancer, kidney cancer, pancreatic cancer, stomach cancer, or a combination thereof. In various instances, the plurality of labeled expression data sets comprises expression data for a subtype of one or more of the labeled cancer type(s), optionally, a subtype of breast cancer. For example, the subtype for breast cancer is, in some aspects, luminal breast cancer, triple negative breast, or a combination thereof. Optionally, the plurality of labeled expression data sets comprises expression data for lung adenocarcinoma, melanoma, renal cell carcinoma, bladder cancer, mesothelioma, and lung small cell cancer.

In exemplary embodiments, each labeled expression data set further comprises data from images, image features, clinical data, epigenetic data, pharmacogenetic data, metabolomics data, or a combination thereof. In exemplary embodiments the labeled expression data comprises RNA expression data, optionally, mRNA expression data. In some aspects, the mRNA expression data is RNA-seq data, optionally, normalized RNA-seq data.

In exemplary instances, the labeled PD-L1 expression status is based on an reverse phase protein array (RPPA) data, fluorescence in situ hybridization (FISH) data, immunohistochemistry (IHC) data, or a combination thereof, optionally, wherein the trained PD-L1 predictive model correlates the labeled PD-L1 expression status with select labeled expression data and/or labeled features.

In exemplary embodiments, the unlabeled expression data set is similar to the labeled expression data set, except that the unlabeled expression data set does not comprise PD-L1 expression status of the subject's sample. In exemplary embodiments, the unlabeled expression data set comprises data from images, image features, clinical data, epigenetic data, pharmacogenetic data, metabolomics data, or a combination thereof, of the subject. In some aspects, the unlabeled expression data set for the sample comprises RNA expression data, optionally, mRNA expression data. In some aspects, the mRNA expression data is RNA-seq data, optionally, normalized RNA-seq data.

In exemplary aspects, the trained PD-L1 predictive model has been trained by (i) inputting a plurality of labeled expression data sets, wherein each labeled expression data set comprises a labeled cancer type and a labeled PD-L1 expression status, and, optionally, one or more labeled features. In exemplary embodiments, the trained predictive model has been trained with a plurality of labeled expression data sets, each labeled expression data set comprises one or more labeled features, and the trained PD-L1 predictive model has been trained according to select labeled features pre-determined to have an association with a phenotype of biological relevance. In exemplar aspects, the trained PD-L1 predictive model was trained using a clustering algorithm to determine which labeled features associate with the phenotype of biological relevance. In some instances, the phenotype of biological relevance is PD-L1 expression status. In alternative or additional aspects, the at least one or more of the select labeled features comprises expression data for at least one gene selected from the group consisting of CD274, TIGIT, CXCL13, IL21, FASLG, TFPI2, GAGE12C, POMC, PAX6, NPHS1, HLA-DPB1, PDCD1, PDCD1LG2, and other genes obtained from the feature selection process. In other aspects, the gene list includes IFNG, GZMB, CXCL9, TGFB1, VIM, STX2, ZEB2, and other genes found via literature search. Optionally, the trained PD-L1 predictive model is a logistic regression model, a random forest model, or a support vector machine (SVM) model, optionally, wherein the logistic regression model is a single-gene or multi-gene logistic regression model.

With regard to the methods of the present disclosure, the methods may include additional steps. For example, the method may include repeating one or more of the recited step(s) of the method. Accordingly, in exemplary aspects, the method comprises re-determining a ratio RS. In exemplary aspects, the method comprises aligning the unlabeled expression data set to labeled expression data according to a trained PD-L1 predictive model every 2, 3, 6, or 12 months, as needed. The method in some aspects further comprises one or more of: obtaining the sample from a subject, isolating mRNA from cells of the sample, fragmenting the mRNA, producing double-stranded cDNA based on the mRNA fragments, carrying out high throughput, short-read sequencing on the cDNA, aligning the sequences to a reference genome, and normalizing raw RNA-seq data.

In some aspects, the method further comprises generating a clinical decision support information (CDSI) report including at least the subject's identity and the identified PD-L1 expression status, and, optionally, providing the CDSI report to a healthcare provider for use in selecting a candidate therapy based on the identified PD-L1 expression status for the subject's sample. Optionally, the high throughput, short-read sequencing is next generation sequencing (NGS), optionally, wherein the NGS comprises hybrid capture. In various instances, the hybrid capture comprises use of biotinylated probes which bind to specific target nucleotide sequences. In exemplary aspects, at least one of the target nucleotide sequences encodes PD-L1, PD-1, or a combination thereof. Alternatively or additionally, at least one of the target nucleotide sequences encodes 4-1BB, TIM-3, or other immune checkpoint molecules.

Any and all possible combinations of the steps described herein are contemplated for purposes of the presently disclosed methods.

The following discussion is given merely to illustrate the present disclosure and not in any way to limit its scope.

FIG. 5A illustrates a process 1101 of PD-L1 prediction using an exemplary PD-L1 predictor 1115. As illustrated in FIG. 5A, a genetic sequence analysis technique may be used to detect RNA molecule copies of a plurality of genes, known as transcripts, in a sample of cancer cells collected from a patient. RNA sequencing (RNA-seq), also known as whole transcriptome shotgun sequencing (WTSS), is a powerful technique that utilizes next-generation sequencing to identify the presence and quantity of RNA in a biological sample at a particular timepoint. RNA-seq is useful for determining and analyzing the ever-changing transcriptome of a cell or tissue. This technique can identify alternatively spliced transcripts, post-transcriptional modifications, gene fusions, mutations or single nucleotide polymorphisms (SNPs) and changes in gene expression over a given time period (e.g., over disease progression or disease regression) and/or upon different treatments (e.g., treatment with one therapeutic agent vs. another vs. no treatment). RNA-seq also allows for the determination of exon/intron boundaries and annotated 5′ and 3′ gene boundaries. In an exemplary embodiment of RNA-seq, messenger RNA (mRNA), produced in vivo by an organism, is extracted from the organism, fragmented, and in vitro copied into double-stranded complementary DNA (ds-cDNA), which is then sequenced using high-throughput, short-read sequencing methods. The sequences are then aligned to a reference genome, obtained from public reference databases, in silico to identify the regions of the organism's genome that were transcribed at the time the mRNA was extracted from the organism.

Still with reference to FIG. 5A, the count for each gene, which is the number of detected transcripts, is stored as RNA transcriptome sequencing (RNA-seq) data. In the example shown, these data are referred to as raw RNA-seq data 1105.

The genetic sequence analysis technique may be biased in a way that causes counts for certain genes to be higher than others, depending on factors which include the length of the gene, the depth setting of the sequence analyzer used in the sequence analysis technique, and the percentage of the gene that contains guanine (G) or cytosine (C), compared to adenine (A) or thymine (T). These biases may cause artifacts, which means that counts for a certain gene would be an inaccurate reflection of the number of transcripts of that genes that actually exist in a sample. An RNA bioinformatics pipeline software 1110 generates normalized RNA-seq data by aligning and adjusting the counts for each gene in raw RNA-seq data 1105 to counteract any artifacts caused by the genetic sequence analysis technique.

In the illustrated example, the PD-L1 status predictor 1115 that receives an input case data set 1112 associated with a cancer cell sample and predicts the PD-L1 status 1120 of the cancer cell sample. In an example, the PD-L1 status predictor 1115 is pan-cancer, meaning that the cancer cell sample receiving a predicted PD-L1 status 1120 may have been collected from a patient with any type or subtype of cancer. For example, cancer types may include brain, lung, breast, colorectal, pancreatic, liver, stomach, skin, etc. and cancer subtypes may include any sub-group within each cancer type, including luminal breast, triple negative breast and other subtypes known in the art. In this example, an input case data set 1112 includes normalized RNA-seq data. The predicted PD-L1 status 1120 may be included in a report 1125. The report 1125 may include a printed report on paper, an electronic document, or a tab or page accessed through an online portal.

The patient or a medical professional 1130, including a physician, nurse, or other trained medical professional, may access the report 1125 and make a case management decision 1135, based in part on the predicted PD-L1 status 1120. A case management decision 1135 may include ordering a test of the cancer cells (for example, IHC, FISH, RPPA, etc.), prescribing a treatment, or another medical action (for example, an action that aims to eliminate or slow the progression of a patient's cancer). For instance, if the predicted PD-L1 status 1120 is indeterminate or equivocal, a physician may order a follow-up test to determine the actual PD-L1 status of the patient's cancer (for example, IHC, FISH, RPPA, etc.). PD-L1 status is used in treatment determinations in accordance with clinical and scientific guidelines (for example, U.S. Food and Drug Administration or National Cancer Care Network guidelines). (See, https://www.cancernetwork.com/nccn/nccn-updated-nscic-guidelines-hone-pd-l1-testing and http://theoncologypharmacist.com/top-issues/2019-issues/may-2019-vol-12-no-2/17752-immunotherapy-strategies-in-the-updated-nccn-guideline-for-nscic-hinge-on-pd-l1-testing) For example, if the predicted PD-L1 status 120 is positive, the physician may decide to make treatment decisions as if the patient sample received a positive PD-L1 result from a PD-L1 test (for example, IHC, FISH, RPPA, etc.). Thus, in various embodiments, a physician may prescribe checkpoint blockade therapies depending on PD-L1 thresholds. For instance, if the PD-L1 biomarker threshold is 50% of cells staining positive for PD-L1 and the patient specimen exceeds this threshold, the physician may prescribe a checkpoint blockade as a patient's first line therapy. If more than 1% of the patient's cells have stained positive for PD-L1 and the patient has failed first line therapy, the physician may prescribe a checkpoint blockade as a second line of therapy.

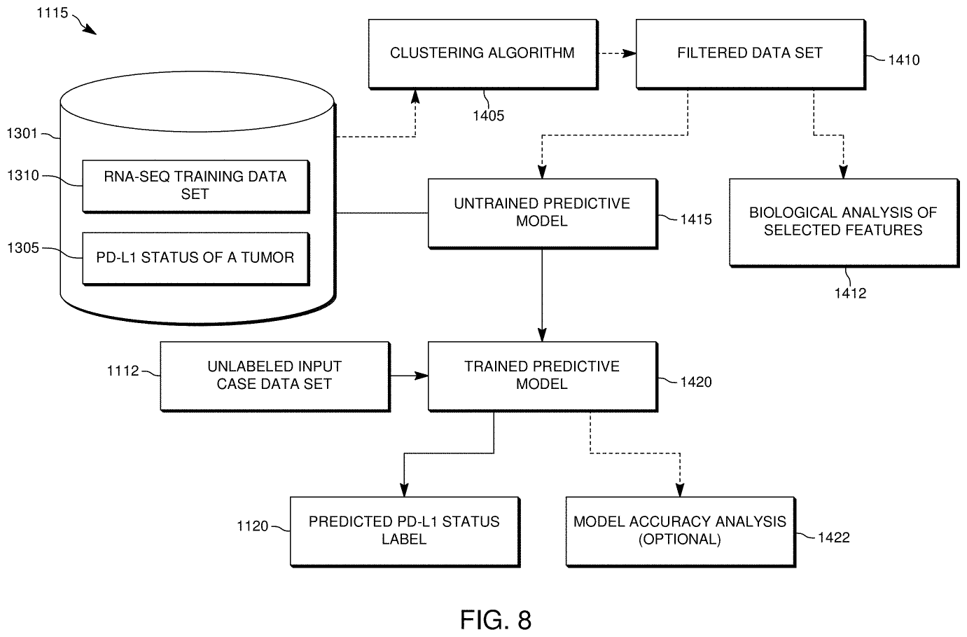

The PD-L1 status predictor 1115 may be a predictive model and/or may be a machine learning algorithm, including random forest, support vector machine (SVM), or logistic regression models. In one example, the PD-L1 status predictor 1115 is a single gene or multi-gene logistic regression model (see FIG. 8). The model software of the PD-L1 status predictor 1115 may be encoded in a docker container such that the software may be run on any platform or operating system.

In this example, the genetic sequence analysis technique may be a next generation sequencing (NGS) assay. The NGS assay may require the preparatory steps of isolating RNA molecules from a patient sample of cells to create a liquid solution containing RNA molecules, measuring the concentration of the RNA molecules in the liquid solution, measuring the average length of the RNA molecules, shortening the RNA molecules if necessary, and creating and collecting DNA copies of the RNA molecules with hybrid capture. Then, the NGS device receives the DNA copies, then detects and reports short-read nucleotide sequences within the DNA copies. In another example, the NGS device may detect and report long-read sequences. Hybrid capture may utilize biotinylated probes to bind to specific target nucleotide sequences within the nucleic acid molecules (DNA copies or RNA) to amplify and collect nucleic acid molecules containing those targeted nucleotide sequences.

Each detected, reported sequence is called a read. The NGS device reports each read that it detects and the number of times (counts) that it detects each read. This report is referred to as raw RNA-seq data 1105. The detected sequence of each DNA molecule copy corresponds to a sequence in an RNA molecule from which that DNA molecule was copied.

The counts in the raw RNA-seq data 1105 for each detected sequence may not reflect the actual number of nucleic acids in the patient sample that contain that sequence, due to artifacts that may be caused by steps in the genetic sequence analysis technique. For example, hybrid capture and amplification may be more likely to create copies of sequences that contain a certain percentage of guanine (G) and cytosine (C) nucleotides, versus adenine (A) and thymine (T) nucleotides. These detected sequence counts may be higher than is expected for the actual number of molecules in the sample that contain these sequences. Other factors that may cause artifacts include the length of a gene from which the sequence is copied and sequencing depth.

The RNA bioinformatics pipeline software 1110 receives the raw RNA-seq data 1105 and determines the most likely location of each read within the entire human genome by comparing the read to a reference genome. The RNA bioinformatics pipeline software 1110 also adjusts the count of each sequence to counteract the effect of any artifacts or biases introduced by the sequence analysis method. This process may be referred to as normalization. Methods of normalizing gene expression data are disclosed in U.S. Provisional Patent Application No. 62/735,349, which is incorporated by reference in its entirety.

Although the systems and methods disclosed herein have been described with specificity for PD-L1, it should be understood that the status of other proteins may be predicted using similar analysis. Other proteins include, but are not limited to, 4-1BB, T-cell immunoglobulin and mucin-domain containing-3 (TIM-3), other immune checkpoint molecules, human epidermal growth factor receptor 2 (HER2), estrogen receptor (ER), and progesterone receptor (PR or PgR). Additionally, this system may be used to predict whether a patient will respond to immune checkpoint blockade therapy and/or another type of cancer treatment.

FIG. 5B illustrates an exemplary predicted PD-L1 status 1120 as it may appear on a report 1125. In this example, the report 1125 is associated with a cancer sample, which is further associated with a predicted PD-L1 status 1120 presented in the report. In this example, the predicted PD-L1 status 1120 is negative, versus equivocal or positive. The report 1125 may further include the CD274 expression level value detected in the associated cancer cell sample. In this example, the cancer sample CD274 expression level value is 0.60.

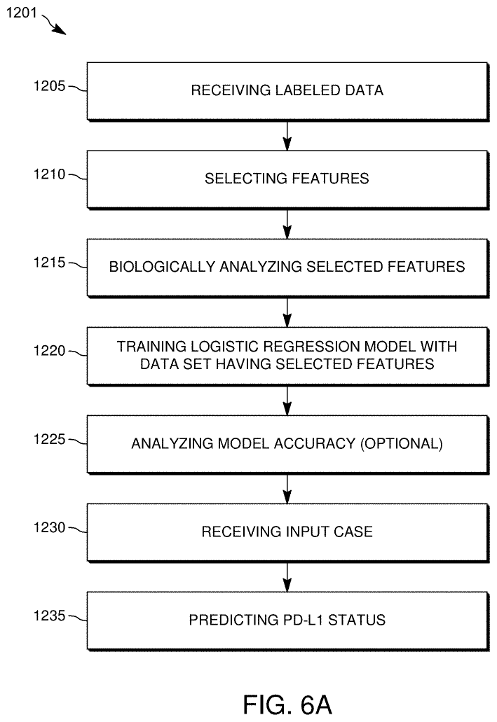

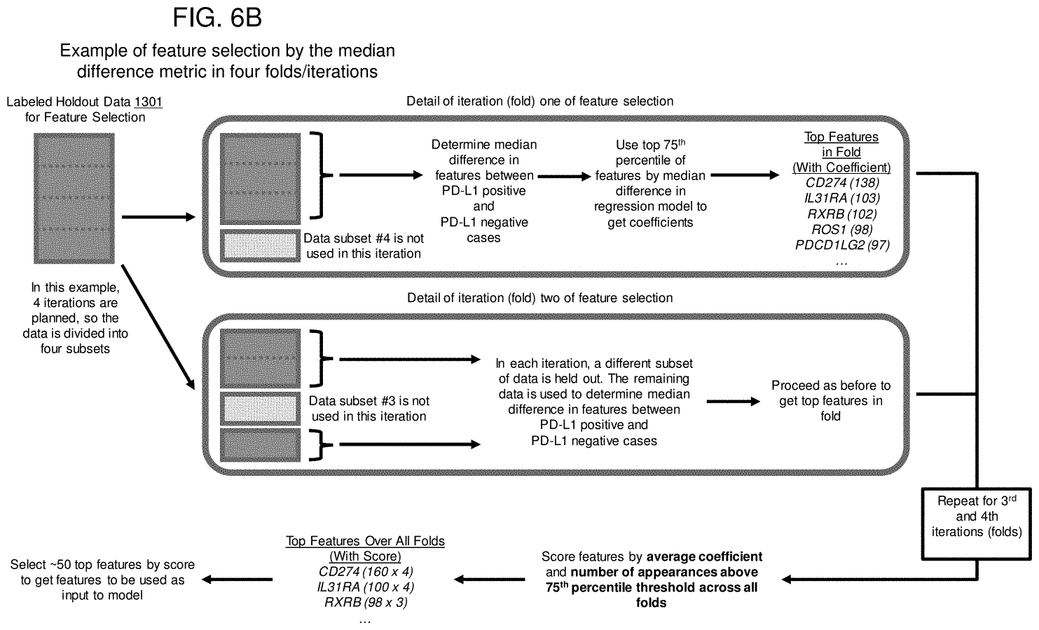

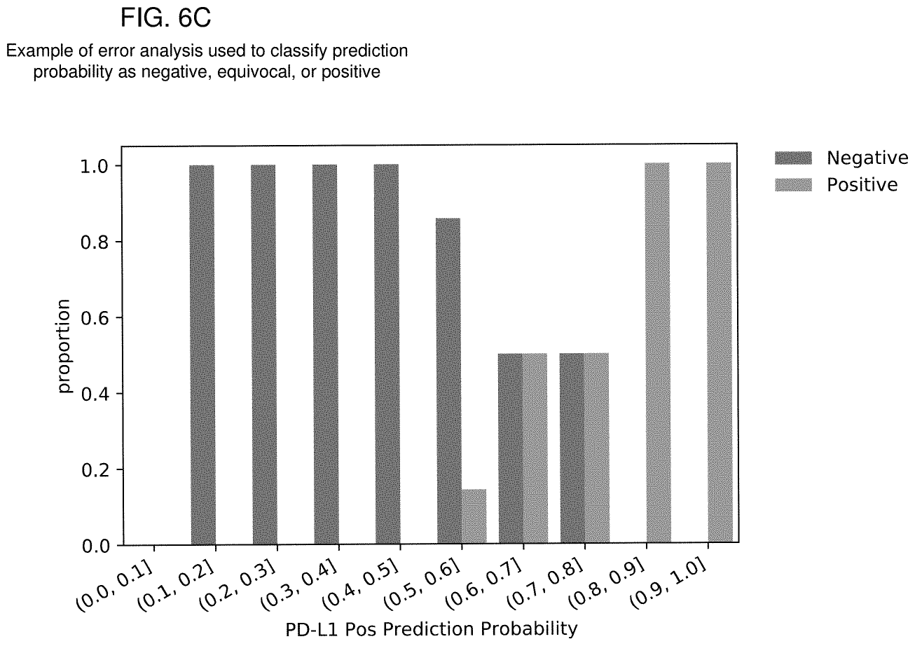

Predicted PD-L1 status 1120 may appear as an additional predicted label associated with a prediction probability 1122 alongside text describing the predictor and implications of the prediction probability. In this example, the PD-L1 positive prediction probability value 1122, which is 0.24, is a numerical output of the PD-L1 predictor 1115, and is a value in a range of approximately 0 through 1 that indicates the probability that the cancer sample is positive for PD-L1. An error analysis may be used to determine the correlation between the PD-L1 positive prediction probability value 1122 and qualitative predicted PD-L1 status 1120, including negative, equivocal, or positive. (See FIG. 6A)

The report 1125 may further include a cancer CD274 histogram 1140a depicting the distribution of CD274 expression levels detected in cancer samples having the same cancer type as the cancer sample associated with the report 1125. The report 1125 may further include a normal CD274 histogram 1140b depicting the distribution of CD274 expression levels detected in a plurality of normal tissue samples. The normal tissue may be of a tissue type that specifically corresponds to the cancer type associated with the cancer sample.

For example, a reference set of non-cancerous skin samples may be used as a reference for a patient with melanoma. These may be visualized together using histograms or other plots. The cancer CD274 histogram 1140a and the normal CD274 histogram 1140b may be located in such a way that represents the relationship between the ranges of expression level values represented by the two histograms. In this example, the majority of the cancer CD274 histogram 1140a is located to the right of the majority of the normal CD274 histogram 1140b with some overlapping to indicate that the cancer CD274 histogram 1140a represents a higher range of values than the normal CD274 histogram 1140b.

The report 1125 may further include a patient CD274 expression level indicator 1140c demonstrating the approximate location of the cancer sample CD274 expression level within the range of expression level values represented by the histograms 1140a and/or 1140b. In this example, the patient CD274 expression level indicator 1140c is located near the right edge of the normal CD274 histogram 1140b and the left edge of the cancer CD274 histogram 1140a. This location of patient CD274 expression level indicator 1140c represents that the cancer sample CD274 expression level value is greater than the majority of the CD274 expression level values represented by the normal CD274 histogram 1140b, and less than the majority of the CD274 expression level values represented by the cancer CD274 histogram 1140a. Alternatively, a report 1125 may include percentile values indicating the percentile of normal tissue or cancer sample CD274 expression level value ranges in which the detected CD274 expression level value lies.

In another example, the report 1125 further includes treatment recommendations based on predicted PD-L1 status 1120 and information from the patient's RNA-seq data 1310, genetic data 1330 and medical record 1325, including treatments that the patient has previously received and any recorded response or change in the health of the patient after the treatment was received. These treatments may include immune checkpoint blockade therapy.

In another example, the histograms 1140a and 1140b, indicator 140c, and the numerical expression level value shown may indicate the distribution of expression levels for another gene, especially if PD-L1 is not the treatment-related molecule of interest for which a presence status is predicted by predictor 1115.

In another example, the predicted PD-L1 status label 1120 may be “positive for PD-L1”, “negative for PD-L1”, or “uncertain/equivocal/testing recommended” and may include a recommendation that the cancer cell sample be tested by IHC, FISH, and/or RPPA to detect PD-L1 proteins.

In one example, the predicted PD-L1 status label 1120 may include the percentage of cancer cells in the cancer cell sample that are predicted to stain positive for PD-L1. If the predicted percentage of cancer cells that stain positive for PD-L1 is less than a selected threshold value, the predicted PD-L1 status is negative. If the predicted percentage of cancer cells that stain positive for PD-L1 is greater than a selected threshold value, the predicted PD-L1 status is positive. In one example, if the predicted percentage of cancer cells that stain positive for PD-L1 is approximately equal to a selected threshold value, or within a selected range of values, the predicted PD-L1 status is uncertain. In one example, the selected threshold value is 1% and the selected range is 1-5%. In another example, the selected threshold value is between 0.1% and 1%. In another example, the selected threshold value is between 0.01% and 0.1%.

FIG. 6A illustrates a method 1201 for training a PD-L1 status predictor 1115 and predicting a PD-L1 status label using a PD-L1 status predictor 1115.

The PD-L1 status Predictor 1115 may include gradient boosting models, random forest models, neural networks (NN), regression models, Naive Bayes models, or machine learning algorithms (MLA). A MLA or a NN may be trained from a training data set such as a plurality of matrices having a feature vector for each patient or images and features. In an exemplary prediction profile, a training data set may include imaging, pathology, clinical, and/or molecular reports and details of a patient, such as those curated from an EHR or genetic sequencing reports. The training data may be based upon features such as the objective specific sets disclosed in FIGS. 6A and 6B. MLAs include supervised algorithms (such as algorithms where the features/classifications in the data set are annotated) using linear regression, logistic regression, decision trees, classification and regression trees, Naïve Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines. NNs include conditional random fields, convolutional neural networks, attention based neural networks, deep learning, long short term memory networks, or other neural models where the training data set includes a plurality of tumor samples, RNA expression data for each sample, and pathology reports covering imaging data for each sample. While MLA and neural networks identify distinct approaches to machine learning, the terms may be used interchangeably herein. Thus, a mention of MLA may include a corresponding NN or a mention of NN may include a corresponding MLA unless explicitly stated otherwise.

An example training data set may be constructed as follows:

| | | | Expression | (Optional) |
|---|---|---|---|---|
| Sample | (optional) | PD-L1 | Level Value | Expression Level |
| ID | Cancer Type | Status | for Gene 1 | Value for Gene 2 |
| 1 | lung | positive | 1.28046675 | . . . |
| 2 | breast | negative | 0.715290684 | . . . |
| 3 | brain | positive | 1.543239819 | . . . |
| . . . | . . . | . . . | . . . | . . . |

In various embodiments, the PD-L1 status is binarized. For example, a positive status is recorded as a 1 and a negative status is recorded as a 0.

In various embodiments, the PD-L1 status reflects the result of a PD-L1 test. In various embodiments, the PD-L1 status is positive if 50% or more of the cancer cells in a histopathology slide stain positive for PD-L1. In various embodiments, the PD-L1 status is positive if 1% or more of the cancer cells in a histopathology slide stain positive for PD-L1.

Training may include providing optimized datasets as a matrix of feature vectors for each patient, labeling these traits as they occur in patient records as supervisory signals (for example, PD-L1 positive or PD-L1 negative), and training the MLA to predict an objective/target pairing. Artificial NNs are powerful computing models which have shown their strengths in solving hard problems in artificial intelligence. They have also been shown to be universal approximators (can represent a wide variety of functions when given appropriate parameters). Some MLA may identify features of importance and identify a coefficient, or weight, to them. The coefficient may be multiplied with the occurrence frequency of the feature to generate a score, and once the scores of one or more features exceed a threshold, certain classifications may be predicted by the MLA. A coefficient schema may be combined with a rule based schema to generate more complicated predictions, such as predictions based upon multiple features. For example, ten (10) key features may be identified across different classifications. A list of coefficients may exist for the key features, and a rule set may exist for the classification. A rule set may be based upon the number of occurrences of the feature, the scaled weights of the features, or other qualitative and quantitative assessments of features encoded in logic known to those of ordinary skill in the art. In other MLA, features may be organized in a binary tree structure. For example, key features which distinguish between the most classifications may exist as the root of the binary tree and each subsequent branch in the tree until a classification may be awarded based upon reaching a terminal node of the tree. For example, a binary tree may have a root node which tests for a first feature. The occurrence or non-occurrence of this feature must exist (the binary decision), and the logic may traverse the branch which is true for the item being classified. Additional rules may be based upon thresholds, ranges, or other qualitative and quantitative tests. While supervised methods are useful when the training dataset has many known values or annotations, the nature of EMR/EHR documents is that there may not be many annotations provided. When exploring large amounts of unlabeled data, unsupervised methods are useful for binning/bucketing instances in the data set. A single instance of the above models, or two or more such instances in combination, may constitute a model.

### Features

A subset of features may comprise molecular data features, such as genomic features derived from RNA features (for example, gene expression levels) or DNA features.

Another subset of features, imaging features may comprise features identified through review of a specimen by pathologist, such as, e.g., a review of stained H&E or IHC slides. As another example, a subset of features may comprise derivative features obtained from the analysis of the individual and combined results of such feature sets. Features derived from DNA and RNA sequencing may include genetic variants, which can be identified in a sequenced sample. Further analysis of the genetic variants may include steps such as identifying single or multiple nucleotide polymorphisms, identifying whether a variation is an insertion or deletion event, identifying loss or gain of function, identifying fusions, calculating copy number variation, calculating microsatellite instability, calculating tumor mutational burden, or other structural variations within the DNA and RNA. Analysis of slides for H&E staining or IHC staining may reveal features such as tumor infiltration, programmed death-ligand 1 (PD-L1) status, human leukocyte antigen (HLA) status, or other immunology-related features.

Features derived from structured, curated, and/or electronic medical or health records may include clinical features such as diagnosis, symptoms, therapies, outcomes, patient demographics such as patient name, date of birth, gender, ethnicity, date of death, address, smoking status, diagnosis dates for cancer, illness, disease, diabetes, depression, other physical or mental maladies, personal medical history, family medical history, clinical diagnoses such as date of initial diagnosis, date of metastatic diagnosis, cancer staging, tumor characterization, tissue of origin, treatments and outcomes such as line of therapy, therapy groups, clinical trials, medications prescribed or taken, surgeries, radiotherapy, imaging, adverse effects, associated outcomes, genetic testing and laboratory information such as performance scores, lab tests, pathology results, prognostic indicators, date of genetic testing, testing provider used, testing method used, such as genetic sequencing method or gene panel, gene results, such as included genes, variants, expression levels/statuses, or corresponding dates associated with any of the above.

Features may be derived from information from additional medical or research based Omics fields including proteome, transcriptome, epigenome, metabolome, microbiome, and other multi-omic fields. Features derived from an organoid modeling lab may include the DNA and RNA sequencing information germane to each organoid and results from treatments applied to those organoids. Features derived from imaging data may further include reports associated with a stained slide, size of tumor, tumor size differentials over time including treatments during the period of change, as well as machine learning approaches for classifying PDL1 status, HLA status, or other characteristics from imaging data. Other features may include additional derivative features sets derived using other machine learning approaches based at least in part on combinations of any new features and/or those listed above. For example, imaging results may need to be combined with MSI calculations derived from RNA expressions to determine additional further imaging features. Other features that may be extracted from medical information may also be used. There are many thousands of features, and the above-described types of features are merely representative and should not be construed as a complete listing of features.

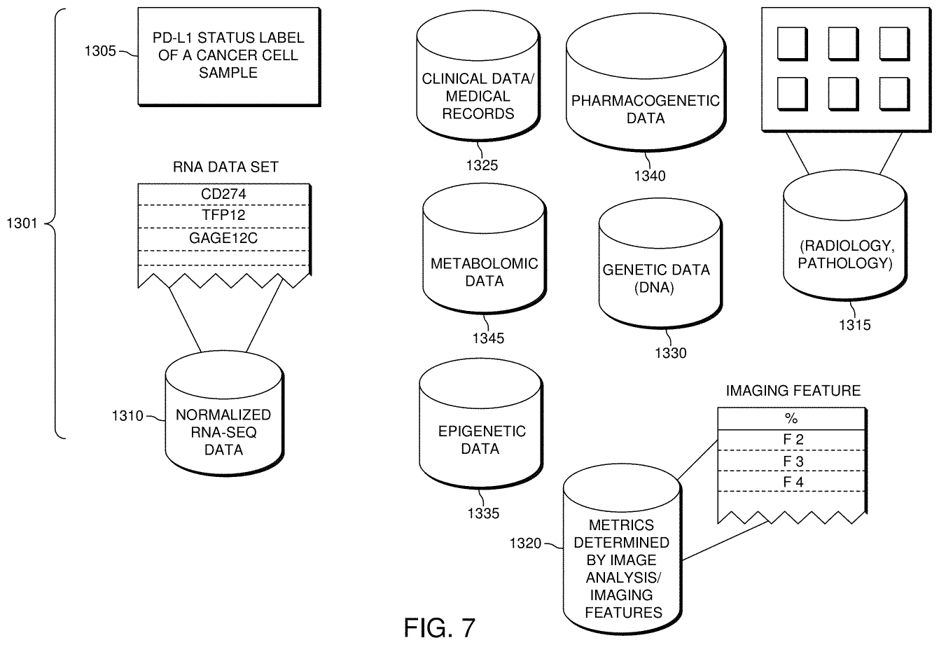

Returning to FIG. 6A, Step 1205 is the step of receiving a labeled data set. The labeled data set 1301 may be associated with multiple cancer cell samples, wherein each cancer cell sample is associated with a positive or negative PD-L1 status label 1305, as determined by IHC, FISH, and/or RPPA (See FIG. 7). The labeled data set 1301 may be a pan-cancer data set, meaning that each cancer cell sample may be collected from a patient with any type or subtype of cancer, and many cancer types and subtypes may be represented by a single labeled data set 1301. The labeled data set 1301 may be further associated with an RNA expression level data set 1310, which may be a normalized RNA-seq data set, images 1315, including radiology and pathology images; imaging features 1320, which include patterns in images 1315 or metrics determined by analyzing images 1315; clinical data 1325, including data extracted from medical records; genetic data 1330 related to DNA molecules contained in the cancer cell sample; epigenetic data 1335; pharmacogenetic data 1340; and metabolomic data 1345.

The curation and assembly of this labeled dataset 1301 can pose obstacles. For example, the ideal RNA expression dataset 1310 associated with the PD-L1 status labels 1305 may involve obtaining IHC tissue from the same tissue sample that is used for nucleic acid isolation and RNA-seq for maximum concordance between the dataset 1310 and the PD-L1 IHC label 1305. Furthermore, a large input RNA dataset needs to be appropriately normalized to allow internal comparisons within a sample and among different samples in terms of gene expression levels. Other obstacles include collecting a large enough specimen through biopsy or blood draw that contains enough cancer cells to create a strong assay signal, successfully genetically sequencing the specimen without failing the standard quality checks associated with NGS and other sequence analysis techniques, and processing the raw data through a bioinformatics pipeline before normalization.

Step 1210 is the optional step of selecting features. A feature is any type of data in the labeled data set that may be correlated with a positive or negative PD-L1 status label 1305. Selected features have been ranked by a metric as being potentially more informative for predicting PD-L1 status than other features from the entire feature set. (See FIG. 6B)

A clustering algorithm 1405 (FIG. 8) may be used to analyze a labeled data set 1301 to select features and create a filtered data set of only the data associated with those features. The labeled data set 1301 may be adjusted by a variety of calculations before feature selection. A clustering algorithm may include Elastic Net, countclust, Cancer Integration via Multikernel Learning (CIMLR), k-means clustering, principal component analysis (PCA), etc. Elastic Net is available from Scikit-Learn (https://scikit-learn.org/stable/modules/generated/sklearn.linear_model.ElasticNetCV.html), countclust is available from University of Chicago (http://bioconductor.org/packages/release/bioc/html/CountClust.html), and CIMLR is available from Stanford University (https://omictools.com/cimlr-tool).

In one example, features may be selected from one or more published lists of data types observed to be related to PD-L1 protein levels, wherein the data types may include the expression levels of specific genes. Selected features may also be a combination of published data types and data types selected by a clustering algorithm or other method.

In the illustrated example, step 1215 is the optional step of biologically analyzing selected features. This step may determine whether any of the selected features measures a biological phenomenon that affects or is affected by the amount of PD-L1 protein that a cell and/or a cell in proximity to the biological phenomenon will produce. This step may include a gene enrichment analysis, which determines whether a list of genes has a disproportionately high number of genes that are involved in a biological pathway that interacts with PD-L1 protein.

For example, the optional biological analysis 1412 (FIG. 8) may determine whether the protein product of each gene on the selected features list interacts with the PD-L1 protein, affects the expression level values of PD-L1 (for example, by behaving as a transcription factor for the CD274 gene), or is involved in a biological pathway that includes PD-L1 or interacts with the biological pathway that includes PD-L1. The analysis may be performed manually or with the assistance of a computer. In one example of this biological analysis, computer-assisted methods including gene set enrichment analysis (GSEA) and related methods are used to determine the enrichment of the feature list with gene sets of interest, for example a set of genes involved in interferon gamma signaling.

In one example, the cancer type or subtype of each cancer cell sample may affect the expected range of expression levels of the CD274 (PD-L1) gene in that cancer cell sample. Therefore, the cancer type or subtype may affect the CD274 expression level in a cancer cell sample that would correspond to a given IHC-stained cancer cell percentage, for example 1% or 50% of cancer cells on a slide staining positively for PD-L1 protein, or a threshold for declaring a positive PD-L1 status 1305 determined by FISH or RPPA. In this example, the biological analysis of selected features may reveal that a feature or a gene is correlated with a cancer type and/or subtype, and may act as an adjustment factor that serves to scale a CD274 expression level that is specific to one cancer type or subtype to convert it to a universal CD274 expression level that serves to rank CD274 expression levels independently of the cancer cell sample cancer type or subtype.

Step 1220 is the step of training a predictive model with a labeled data set or a filtered data set having only data associated with selected features. For each selected feature, the model may receive data values or data patterns and associates each value or pattern with a probability of being associated with a positive or a negative PD-L1 status label 1305. After this probability association, the trained predictive model can receive an unlabeled input case 1112 associated with a cancer cell sample and assign a predicted PD-L1 status label 1120 to the associated cancer cell sample based on the data values or patterns included in unlabeled input case 1112 and the probabilities associated with each data value or data pattern.

In one example, the data values are mean centered and rescaled before model training. To mean center the expression level values in the labeled data set 1301 for one gene, a mean for that gene may be calculated by averaging the expression level values of all cancer cell samples for that gene, and adjusting the expression level value for each cancer cell sample by subtracting that gene's mean from the expression level value of that gene in each cancer cell sample. Then, the adjusted expression level values may be rescaled by multiplying each adjusted value by a factor k, wherein k is selected so that the standard of deviation of the adjusted, rescaled expression level values from all cancer cell samples, for that gene, equals a selected value. In one example, the selected standard of deviation value is set to one. In one example, these calculations are done using Scikit-learn tools, including as the StandardScaler (https://scikit-learn.org/stable/modules/generated/sklearn.preprocessing.StandardScaler.html).

The predictive model may be a logistic regression model that includes a logistic regression function and logistic regression model coefficients. The logistic regression function may be calculated based on the filtered data set or the labeled data set. In one example, the logistic regression model coefficients are determined by minimizing a loss function, which can be done using stochastic gradient descent.

A brief sketch of the logistic regression function is detailed here. The binary logistic regression problem uses the sigmoid function to map an input to an interval of (0, 1). In logistic regression and other machine learning applications, this mapping corresponds to prediction probabilities 1122.

Given an intercept x₀, n features x₁, x₂, . . . , xₙ and n feature weights (also known as coefficients) W₁, W₂, . . . , Wₙ logistic regression first calculates a linear combination of weighted features, here denoted z. This result is then used as input into the sigmoid function resulting in a prediction probability 122 for the class. See the embodiments below for examples of feature weight values.

Mathematically, this is written as z=x₀+W₁x₁+W₂x₂+ . . . +Wₙxₙ

The sigmoid function sigmoid: R→(0, 1) then operates on z as follows:

$$sigmoid ( z ) = \frac{1}{1 + {e}^{- z}}$$

The standard definition in binary logistic regression is to use 0.5 as a decision threshold, such that a calculated sigmoid value, which is the predicted probability 1122, of greater than or equal to 0.5 is classified as positive, and a predicted probability 1122 of less than 0.5 is classified as negative.

In one example, classifying the predicted probability 1122 as a type of predicted PD-L1 status label 1120 (negative, equivocal, or positive), includes selecting a first threshold value and a second threshold value, wherein the second threshold value is greater than the first threshold value. Both threshold values may be equal to a value in the range of 0 through 1. If the predicted probability value 1122 is less than the first threshold value, the predicted probability may be classified as negative. If the predicted probability value 1122 is greater than the first threshold value but less than the second threshold value, the predicted probability 1122 may be classified as equivocal, meaning that the PD-L1 predictor 1115 result has very low confidence in reporting a negative or positive predicted PD-L1 status label 1120. Equivocal may also be known as uncertain. If the predicted probability value 1122 is greater than the second threshold value, the predicted probability 1122 may be classified as positive.

In one example, error analysis may be used to manually or automatically select these first and second threshold values. Error analysis may include using the PD-L1 predictor 1115 to generate a predicted probability value 1122 for each input case 1112 from a labeled validation data set. The labeled validation set is associated with at least one input case 1112 comprised of a cancer cell sample, wherein each cancer cell sample is further associated with selected features data and one PD-L1 status label 1305. The error analysis may further include comparing each predicted probability value 122 to the associated PD-L1 status label 1305 to determine the relationship between the predicted probability value 1122 and the PD-L1 status label 1305. (See FIG. 6C) The PD-L1 predictor 1115 does not receive the PD-L1 status labels 1305 to generate the predicted probabilities 1122. The labeled validation data set may not have been previously presented to the PD-L1 predictor 1115 during feature selection and/or training.

In one example, feature weights are found by minimizing a loss function. In logistic regression the loss function may be binary cross-entropy.

Binary cross-entropy is formulated as follows:

$$J ( θ ) = - \frac{1}{m} {\sum }_{i = 1}^{m} [ {y}^{( i )} log ( {h}_{θ} ( {x}^{( i )} ) ) + ( 1 - {y}^{( i )} ) log ( 1 - {h}_{θ} ( {x}^{( i )} ) ) ]$$

where the loss function J is a function of the model parameters including coefficients, y is an indicator variable that takes on the value of 1 or 0, corresponding to whether the predicted class label is the correct classification. In this equation h_{θ}(x) is the prediction probability.

Other common loss functions include Regression Loss Functions, Mean Square Error/Quadratic Loss, Mean Absolute Error (MAE), Huber Loss/Smooth MAE, Log cos h Loss, Quantile Loss, etc. One or more cancer cell samples associated with the labeled data set or filtered data set may be withheld from the model during training.

Step 225 is the optional step of analyzing the accuracy of the trained predictive model. During accuracy analysis, a model may be adjusted to eliminate or add selected features and the model accuracy may be assessed for each list of selected features. Model accuracies may be compared to select a final list of selected features resulting in the highest accuracy.

In one example, the trained predictive model receives at least one withheld cancer cell sample but does not receive or process the PD-L1 statuses 1305 associated with the withheld cancer cell samples. The trained predictive model predicts a PD-L1 status label 1120 for each withheld cancer cell sample. The PD-L1 status 1305 and the predicted PD-L1 status label 1120 associated with each withheld cancer cell sample may be compared to perform a model accuracy analysis 1422.

The model accuracy analysis 1422 may include more than one withheld cancer cell sample, wherein each of the withheld cancer cell samples is associated with a PD-L1 status 1305 and a predicted PD-L1 status label 1120. If the PD-L1 status 1305 and the predicted PD-L1 status label 1120 are both positive, this is a true positive result. If both are negative, this is a true negative result. If the PD-L1 status 1305 is negative and the predicted PD-L1 status label 1120 is positive, this is a false positive result. If the PD-L1 status 1305 is positive and the predicted PD-L1 status label 1120 is negative, this is a false negative result.

The model accuracy analysis 1422 may include plotting a receiver operating characteristic (ROC) curve and computing the area under curve (AUC) for all of the withheld cancer cell sample and/or at least one subset of the withheld cancer cell samples. Evaluation of model performance may also include calculating accuracy, precision (also called positive predictive value), recall (also called sensitivity or true positive rate), specificity (also called true negative rate), false positive rate, adjusted mutual information and other metrics on one or more withheld cancer cell samples. Precision-recall curves may be used for analysis of model precision and recall. Adjusted mutual information measurements may indicate whether the features of the model were selected by random chance or if the combination of the selected features is statistically unlikely to occur randomly. If the grouping of features were unlikely to occur randomly, this may indicate that it is likely that the selected features have been grouped because they share characteristics that can accurately predict PD-L1 status and/or are biologically related to PD-L1 expression.

In one example, accuracy, precision, recall, specificity and a false positive rate may be calculated according to the following formulae, where TP is the number of true positive results; TN is the number of true negative results; FP is the number of false positive results; and FN is the number of false negative results.

Accuracy=(TP+TN)/(TP+TN+FP+FN)

Precision=TP/(TP+FP)

Recall=TP/(TP+FN)

Specificity=TN/(TN+FP)

False Positive Rate=FP/(FP+TN)

Step 1230 is the step of receiving an input case data set 1112. The trained predictive model receives input case data set 1112, which includes data associated with a cancer cell sample. The data in input case data set 1112 may be of the same type as the selected features, if the model was trained on a filtered data set. In one example, the input case data set 1112 does not include a PD-L1 status 1305 associated with the cancer cell sample.

Step 1235 is the step of predicting a PD-L1 status label 1120 associated with the cancer cell sample of the input case data set 1112. The trained predictive model predicts the PD-L1 status label 1120 based on the input case data set 1112. If features were selected, the prediction may be based only on the values in input case data set 1112 that are associated with the selected features.

FIG. 6B illustrates an exemplary method for selecting features for a PD-L1 status predictor. In the example shown, the labeled data set 1301 is a holdout labeled data set, wherein holdout means that the data set will only be used for selecting features and not used for training 1220 or model accuracy analysis 1422 of the PD-L1 predictor 1115. In one example, a labeled data set 1301 used for feature selection is not a holdout data set. In this example, the labeled data set 1301 includes a row for each cancer cell sample and a column containing either a numerical representation of the positive or negative PD-L1 status label 1305 associated that cancer cell sample or a numerical representation of the normalized RNA expression level of a gene, for each gene in the human genome. The normalized RNA expression level of a gene may be the normalized number of counts detected by NGS.

In the example shown, before feature selection, the labeled data set 1301 is divided into four subsets such that each cancer cell sample is assigned to one of the subsets and each subset has approximately the same number of cancer cell samples. The subsets are combined to create four different folds such that fold one is composed of subsets 1, 2, and 3, fold two is composed of subsets 1, 2, and 4, fold three is composed of subsets 1, 3, and 4, and fold four is composed of subsets 2, 3, and 4. Each cancer cell sample may be assigned to more than one fold. Each fold may be a subset of the labeled data set 1301. In another example, each cancer cell sample within the adjusted labeled data set to only one of the folds such that each fold has approximately the same number of cancer cell samples.

In another example not shown here, the Elastic Net algorithm from Scikit-Learn is the clustering algorithm 1405 used to analyze the cancer cell samples in each fold to quantify the correlation of the PD-L1 status 1305 of the cancer cell sample and the values stored for each data type associated with the sample. The clustering algorithm 1405 assigns a weight to each data type, wherein the weight is a numeric value that represents the strength of the correlation between the expression level value of the gene in a cancer cell sample and the PD-L1 status 305 of the cancer cell sample.

For all examples using Elastic Net, parameter tuning may be initialized with a selected value, grid-search, or a default initial value.

Alternatively, in the example shown, the first fold is divided into two subsets: cancer cell samples associated with a positive PD-L1 status 1305 versus cancer cell samples associated with a negative PD-L1 status 1305. The median expression level value is calculated for each gene for each subset. Then, the median difference between the PD-L1 negative median and the PD-L1 positive median is calculated for each gene.

The genes having the highest median differences or median differences greater than a selected threshold value may be selected as features. In one example, the threshold is empirically set at a percentile score that has been selected to ensure that CD274, the feature hypothesized a priori to be most correlated with PD-L1 expression levels, is included in the feature list. In one example, this threshold is defined as the 75th percentile of the group of median difference values observed for all genes in the fold; thus, all features exhibiting a median difference higher than the 75th percentile are selected to produce a list of selected features for that fold.

The clustering algorithm 1405 calculates coefficients, which are also known as weights or selection weights, for each feature and the clustering algorithm 1405 may be Elastic Net. The median difference and feature selection as described is repeated for each fold.

In both examples, the clustering algorithm 1405 assigns a selection weight value to each data type in the fold. The selection weight value reflects the strength of the correlation between each data type and the PD-L1 status 1305 of a cancer cell sample. In one example, the selection weight value is greater if the correlation is stronger. If a data type is a selected feature for more than one fold, the data type is given higher priority to be included in the final list of features for the PD-L1 predictor. In one example, the priority increases for every fold in which the data type has been assigned a weight value that exceeds the fold weight threshold.

Accordingly, the data types are chosen for the final list of selected features and the filtered data set 1410 for training the PD-L1 predictor 1115 based on two values: the average or mean of the selection weight value assigned within each fold to the data type, and the number of folds for which the data type receives a selection weight value that exceeds a threshold. These two values are combined into a score value for each data type. The score for a feature X across k number of folds can thus be defined as

$$score ( X ) ≡ \frac{1}{k} {\sum }_{i = 1}^{k} [ {W}_{X , i} ] \cdot {N}_{X}$$

where W_{X,i} is the selection weight value assigned to the feature X in fold i and N_{X} is the number of times that the feature was above the threshold across k folds. Feature selection methods such as the formulation detailed above can be used as a strategy to improve performance of many predictors based on an improvement of selecting the most informative features.

Dividing the labeled data set 1301 into folds may reduce the likelihood that a gene with naturally variable expression level values that are biologically unrelated to PD-L1 protein levels gets a large score value.

All data types that score above a selected threshold value may be selected as features or the data types may be sorted by score value and any number of the highest-scoring data types may be selected as features. In one example, approximately 40-50 of the highest-scoring data types are selected as features.

These approaches may be applied to the data values of any data type in the labeled data set 1301 to select features and is not limited to gene expression level values.

Multiple embodiments are described below (see FIG. 8), each with a complete exemplary list of selected features and the corresponding exemplary feature weight values in the logistic regression function of the trained predictive model 1420.

FIG. 6C illustrates an analysis used to select thresholds for classifying a PD-L1 prediction probability 1122 as negative, equivocal, or positive. During error analysis, the PD-L1 status label 1305 associated with each cancer cell sample in labeled data set 1301 or a testing data set may be associated with the predicted probability value 1122 generated by the PD-L1 predictor 1115 for that cancer cell sample and plotted to illustrate the relationship between the predicted probability values 1122 and the PD-L1 status label 1305. The testing data set may be identical to labeled data set 1301 except that the data in the testing data set have not been used to train the PD-L1 predictor 1115.

In the error analysis results shown in FIG. 6C, each prediction probability result 1122 is sorted manually or automatically into numeric intervals, based on the prediction probability value 1122. In one example, the automatic sorting is done by the Python pandas.cut function (https://pandas.pydata.org/pandas-docs/version/0.23.4/generated/pandas.cut.html). The boundaries of each interval (indicated on the x-axis) may be manually or automatically selected, and the height of each bar displayed for each interval represents the proportion of predicted probability values 1122 associated with either a negative PD-L1 status label 1305 (blue bars) or a positive PD-L1 status label 1305 (orange bars). In one example, the proportions may be calculated automatically by pandas functions from Python, for example, by using the dataframe methods .groupby, .apply, .value_counts, and .reset_index (https://pandas.pydata.org/). In one example, each interval is called a bin and the negative or positive PD-L1 status labels 1305 are known as the types of errors that occur in each bin.

In this example, the range of possible prediction probability values is 0 to 1 and it is divided equally into ten intervals—(0.0, 0.1], (0.1, 0.2], etc. The heights of the bars in FIG. 6C indicate that prediction probability values 1122 between approximately 0 and 0.6 are mostly associated with negative PD-L1 status labels 1305, prediction probability values 1122 between approximately 0.6 and 0.8 are associated equally with negative and positive PD-L1 status labels 1305, and prediction probability values 1122 between approximately 0.8 and 1 are mostly associated with positive PD-L1 status labels 1305. Accordingly, the first threshold value may be set as 0.6 and the second threshold value may be set as 0.8 such that the prediction probability values 1122 associated with negative PD-L1 status labels 1305 are classified as a negative predicted PD-L1 status 1120, the prediction probability values 1122 equally associated with both positive and negative are classified as an equivocal predicted PD-L1 status 1120, and the prediction probability values 1122 associated with positive PD-L1 status labels 1305 are classified as a positive predicted PD-L1 status 1120.

FIG. 7 illustrates example training data that may be included in a labeled data set 1301, which is used for training the PD-L1 predictor 1115. The PD-L1 predictor 1115 may be trained on a labeled data set 1301 that includes data associated with at least one cancer cell sample, wherein each cancer cell sample is associated with a positive or negative PD-L1 status label 1305, as determined by IHC, and a normalized RNA-seq data set 1310. In another example, instead of, or in addition to, a PD-L1 status label 1305, each cancer cell sample from a patient is further associated with data related to the patient's response to immune checkpoint blockade therapy and/or another type of cancer treatment. These data may include a measurement or score indicating the impact of a treatment on the health of the patient, which may include overall survival time, time until surgery, and/or progression-free survival time after receiving immune checkpoint blockade therapy and/or another type of cancer treatment.

Each normalized RNA-seq data set 1310 may include an expression level value for each gene in the human genome, wherein each expression level value indicates how many RNA copies of that gene are detected in the cancer cell sample. Examples of genes include CD274, TFP12, GAGE12C, etc. In one example, each normalized RNA-seq data set 1310 includes an expression level value for the whole transcriptome, approximately 20,000 human genes.

In one example, the labeled data set 1301 includes more than 500 cancer cell samples, wherein more than 75 of the samples are associated with a positive PD-L1 status 1305 and the remainder of the samples are associated with a negative PD-L1 status 1305.

The PD-L1 status 1305 for all cancer cell samples in the training data set may be determined by FISH, RPPA, or IHC. There is more than one clone, or type, of antibody that can detect PD-L1 for IHC staining. In one example, the PD-L1 status 1305 for all cancer cell samples in the training data set may be determined by IHC staining that utilizes one specific clone of the anti-PD-L1 antibody. In one example, the anti-PD-L1 antibody clone utilized for IHC staining is the clone known as 22C3.

Once the PD-L1 status predictor 1115 (see FIG. 8) is trained, it receives an input case data set 1112 (e.g., from a test patient data store) associated with a cancer cell sample with an unknown PD-L1 status 1305. The input case data set 1112 may include a normalized RNA-seq data 1310 associated with the cancer cell sample.

In the example shown in FIG. 7, the labeled data set 1301 and the input case data set 1112 may further include additional data associated with the cancer cell sample. Additional data may include images 1315, including radiology and pathology images; imaging features 1320, which include patterns in images 1315 or metrics determined by analyzing images 1315; clinical data 1325, including data extracted from medical records; genetic data 1330 related to DNA molecules contained in the cancer cell sample; epigenetic data 1335; pharmacogenetic data 1340; and metabolomic data 1345.

Images 1315 may include 2- or 3-dimensional depictions of solid tumors or other portions of a patient's body affected by cancer, which include radiology images, computed tomography (CT) scans, also known as computerized axial tomography (CAT) scans, including CT angiography; fluoroscopy, including upper GI and barium enema; magnetic resonance imaging (MRI) and magnetic resonance angiography (MRA); mammography; nuclear medicine, which includes such tests as a bone scan, thyroid scan, and thallium cardiac stress test; x-rays; positron emission tomography, also called PET imaging, PET scan, or PET-CT when it is combined with CT; and ultrasounds.

Images 1315 may also include images of pathology slides, also known as histology slides, wherein each slide is a slice of tumor tissue or other cancer cell sample mounted on a microscope slide, that may have been stained by immunohistochemistry (IHC) staining and/or hematoxylin and eosin (H and E), two stains commonly used together to analyze cancer cells.

In this example, the imaging features 1320 may be visual patterns that exist in the pixel data associated with images 1315 and/or metrics calculated by analyzing the images 1315. For example, the imaging features 1320 may include the volume of a tumor, the degree of immune infiltration in a tumor, or the tumor purity percentage of a cancer cell sample. Other types of imaging features that may be incorporated are described in U.S. Provisional Patent Application No. 62/693,371, which is incorporated herein in its entirety.

In one example, labeled data set 1301 only includes a PD-L1 status 1305 and a normalized RNA-seq data 1310 for each cancer cell sample associated with labeled data set 1301.

FIG. 8 illustrates an example PD-L1 status predictor 1115.

In the example shown, a PD-L1 status predictor 1115 includes a labeled data set 1301 that may be processed by a clustering algorithm 1405 to select features and create a filtered data set 1410, which contains the data associated with selected features.

An untrained predictive model 1415 receives the filtered data set 1410 or the labeled data set 1301 to create a trained predictive model 1420, a process known as model training. In one example, each cancer sample associated with the filtered data set 1410 or the labeled data set 1301 is further associated with a data value or data pattern of each selected feature data type and a PD-L1 status label 1305. The PD-L1 status label 1305 may be positive, negative, or uncertain, and it may include a value indicating the percentage of cancer cells that stained positive for PD-L1 protein, which indicates that those cells had produced PD-L1 protein.

During model training, for each selected feature in the filtered data set 1410 or the labeled data set 1301, the model receives data values or data patterns and associates each value or pattern with the positive or negative PD-L1 status label 1305 associated with a cancer cell sample having that data value or pattern for that data type. After this probability association, the trained predictive model 1420 can receive an unlabeled input case 1112 having data values or data patterns for each selected feature, and assign a predicted PD-L1 status label 1120 to the associated cancer cell sample based on the data values or patterns in unlabeled input case 1112 and the probabilities associated with each data value or data pattern.

In one example, trained predictive model 1420 may be a trained logistic regression model that includes a logistic regression function and logistic regression model coefficients. The logistic regression function may be calculated based on the filtered data set 1410 or the labeled data set 1301. The logistic regression model coefficients may be determined by minimizing a loss function as described above.

In one example, during training, the untrained predictive model 1415 does not receive every cancer cell sample associated with the filtered data set 1410 or labeled data set 1301, and the cancer cell samples that the untrained predictive model 1415 does not receive may be referred to as withheld cancer cell samples.

In one example, the trained predictive model 1420 receives at least one withheld cancer cell sample but does not receive or process the PD-L1 statuses 1305 associated with the withheld cancer cell samples. The trained predictive model 1420 predicts a PD-L1 status label 1430 for each withheld cancer cell sample. The PD-L1 status 1305 and the predicted PD-L1 status label 1120 associated with each withheld cancer cell sample may be compared to perform a model accuracy analysis 1422.

As described above, the model accuracy analysis 1422 may include more than one withheld cancer cell sample, wherein each of the withheld cancer cell samples is associated with a PD-L1 status 1305 and a predicted PD-L1 status label 1120. The model accuracy analysis 1422 may include plotting a receiver operating characteristic (ROC) curve and computing the area under curve (AUC) for all of the withheld cancer cell sample and/or at least one subset of the withheld cancer cell samples.

The trained predictive model 1420 receives and analyzes an input case data set 1112 associated with a cancer cell sample and predicts the PD-L1 status 1120 of that cancer cell sample. In one example, the input case data set 1112 does not include a PD-L1 status 1305 associated with the cancer cell sample. The input case data set 1112 includes data associated with a cancer cell sample, which may include normalized RNA-seq data 310, images 315, imaging features 1320, clinical data 1325, genetic data (DNA) 1330, epigenetic data 1335, pharmacogenetic data 1340, and/or metabolomic data 1345. In one example, the input case data set 1112 includes only RNA-seq data 310 associated with a cancer cell sample.

The predicted PD-L1 status label 1120 of the cancer cell sample may be reported to a physician, medical professional, or patient through a software portal, electronic file including a portable document format (PDF), or hard-copy document, including a document printed on paper. The predicted PD-L1 status 1120 may assist the medical professional in choosing an anti-cancer treatment, such as a treatment that is likely to eliminate the sampled cancer cells. Treatment may be prescribed to the patient in a therapeutically effective amount.

In one example, the predicted PD-L1 status 1120 is a companion diagnostic, meaning that the predicted status may be used to indicate whether a treatment is likely to reduce or eliminate the cancer cells in the patient from which the sample was collected. In one example, the predicted PD-L1 status 1120 is College of American Pathologists (CAP) accredited and/or Clinical Laboratory Improvement Amendments (CLIA) certified. In another example, the predicted PD-L1 status 1120 is FDA-approved. This accreditation, certification and/or approval may be based on the proven accuracy of the predicted PD-L1 status 1120.

### Example Embodiments of the PD-L1 Status Predictor 1115

The following three embodiments of the PD-L1 status predictor 1115 each have a distinct method for selecting features in labeled data set 1301 based on their correlation with PD-L1 status 1305.

### First Embodiment of a PD-L1 Status Predictor 1115

In the first embodiment of a PD-L1 status predictor 115 disclosed here, a type of data may be selected as a feature if it represents a biological condition and/or phenomenon that has a known effect on the amount of PD-L1 protein present in a sample.

For example, the number of RNA copies, called the expression level value, of the CD274 gene may be selected as a feature.

The RNA expression level value of the CD274 gene, as reported in the normalized RNA-seq data 1310, is a selected feature used to train the untrained predictive model 1415. The model training results in a trained predictive model 1420 that correlates CD274 RNA expression values with PD-L1 status 1305 and predicts PD-L1 status 1120 for the cancer cell sample associated with an input case 1112, based on the CD274 expression level value associated with that cancer cell sample.

The trained predictive model 1420 may include a logistic regression function calculated based on the CD274 expression level values included in the filtered data set 1410 or the labeled data set 1301. As described above, the logistic regression model coefficients may be determined by minimizing a loss function.

In this embodiment, the normalized RNA-seq data set 1310 for each sample included in the labeled data set 1301 or an input case 1112 includes an expression level value for the CD274 gene.

### Second and Third Embodiments of a PD-L1 Status Predictor 1115

In the second and third embodiments of a PD-L1 status predictor 1115 disclosed here, the selected features include expression level values of genes in the labeled data set 1301 that are most closely correlated with a cancer cell sample's PD-L1 status 1305. The data values in the labeled data set 1301 are adjusted before a clustering algorithm 1405 analyzes the labeled data set 1301 to select the features for each of these embodiments. (See FIG. 6B) The adjustments to the data values in the labeled data set 1301 for embodiment two are slightly different than the adjustments for embodiment three, as detailed below. Once adjusted, these data values may be stored as an adjusted labeled data set and the untrained predictive model 1415 may receive the adjusted labeled data set to generate trained predictive model 1420. The filtered data set 1410 may be generated from the adjusted labeled data set.

In embodiment two, the median difference model, a feature is defined as having a correlation with the PD-L1 status label 1305 if it has a large difference value, wherein the difference value is calculated by subtracting the median value of the set of data values associated with a positive PD-L1 status 1305 from the median value of the set of data values associated with a negative PD-L1 status 1305. (See FIG. 6B) The data sets used are drawn from holdout data specifically set aside for median difference calculation or feature selection. A holdout data set may be any subset of the labeled data set 1301. Any subset of the labeled data set 1301 that is not associated with the holdout data set may be prevented from being used for feature selection 1210 or model training 1220 and used only to test the model during the optional model accuracy analysis 1422.

In one example, (embodiment two) the selected features include expression level values for the following genes: immune checkpoint genes PD-L1, PD-L2, and TIGIT; chemokine and cytokine genes CXCL13, and IL21; and immune activity-related gene FASLG. In another example, (embodiment three) the selected features include expression level values for the following genes: CD274, TFPI2, GAGE12C, POMC, PAX6, NPHS1, and HLA-DPB1.

In this embodiment, the trained predictive model 1420 may include a logistic regression function calculated based on the data values or data patterns associated with selected features included in the filtered data set 1410 or the labeled data set 1301. As described above, the logistic regression model coefficients may be determined by minimizing a loss function.

In one example, (embodiment two) all selected features are expression level values of genes, including but not limited to the following genes: CD274 (2.238), IL31RA (−0.212), RXRB (0.298), NCF1 (0.264), NKX2-8 (0.326), MYEOV (−0.086), IL21 (−0.415), ZBED2 (−0.379), PSG4 (−0.092), ROS1 (0.270), PDCD1LG2 (0.371), IFNL3 (0.141), ACTBL2 (0.024), ANKRD34B (−0.366), KMO (−0.499), HTR1D (−0.171), CCBE1 (0.580), NETO1 (0.071), KLC3 (−0.278), RGS20 (0.259), PRSS36 (−0.143), GTSF1 (−0.043), SPRR1B (0.255), CYP27B1 (−0.007), SDK1 (−0.407), GNGT1 (0.443), COPZ2 (0.043), PSMB8 (−0.437), CR2 (0.202), HLA-DQA1 (0.033), HMGA1 (−0.080), ST6GALNAC5 (0.000), TCHH (−0.094), HLA-DQB1 (0.303), MYBPC2 (0.279), ULBP2 (−0.345), SCGB3A2 (−0.689), TMPRSS4 (−0.496), LIPG (0.373), CARD17 (0.051), HLA-DRB1 (0.325), and AHNAK2 (0.208). The value denoted in parentheses after a gene name indicates the approximate feature weight value for that gene expression level in the logistic regression function.

In embodiment three, the variance model, the features selected to predict the PD-L1 status label 1120 are selected from the set of features that have the highest variance. Elastic Net was used to calculate a coefficient (selection weight value) for each feature and features were selected as described above. (See FIG. 6B) After feature selection each expression level value was mean centered and rescaled as described above, per gene, and stored as an adjusted labeled data set received by the untrained predictive model 1415 as an input into the prediction process to generate trained predictive model 1420.

In one example, (embodiment three) all selected features are expression level values of genes, including but not limited to the following genes: CD274 (2.142), CEACAM21 (0.193), LRRC37A2 (0.207), RNASE10 (0.041), PSG4 (0.318), SYT1 (0.152), HTR1D (0.043), IL31RA (−0.207), TRIM50 (0.160), ANKRD2 (0.647), SLC25A41 (0.201), GAGE12H (0.093), CPA5 (−0.167), GAGE12C (0.103), GAGE12D (0.103), FKBPL (0.695), PSG5 (−0.299), NCF1 (0.372), IL21 (−0.222), CYP2A13 (0.300), NOXO1 (−0.314), ANKRD34B (−0.813), ITPKA (−0.112), HLA-DPB1 (0.422) PSG9 (−0.373), CAMK1G (−0.137), HMGN5 (−0.152), BTG4 (0.267), FGF5 (0.169), KRT24 (−0.166), SAXO2 (−0.278), CLLU1OS (−0.094), KRT31 (−0.358), PAGE1 (0.037), PRM3 (−0.298), LRRC37A (−0.054), ANKRD18B (0.321), PSG2 (0.173), AFAP1L2 (−0.184), and DMRTA2 (0.307). The value denoted in parentheses after a gene name indicates the approximate feature weight value for that gene expression level in the logistic regression function.

The genes on the selected features list may be analyzed to determine whether they have any biological connection to the level of the predicted protein, PD-L1 as described above (see FIG. 6A).

In embodiment two, the following genes selected as features and/or proteins encoded by these genes have these general functions, according to published research: CD274 encodes immune inhibitor PD-L1, IL31RA encodes an immune cytokine receptor, RXRB encodes a retinoic acid receptor and has some immune functions, NCF1 encodes an oxidase for neutrophils and has some immune functions, NKX2-8 is important for liver development and certain cancer types may affect its expression levels, MYEOV has an unclear function but expression levels may vary with tissue and/or cancer type, IL21 encodes an immune cytokine, ZBED2 expression levels may vary with tissue and/or cancer type, PSG4 may regulate the immune system and cell adhesion and is expressed by fetal tissue but certain cancer types may increase expression levels in adults, ROS1 expression levels may be affected by certain cancer types, PDCD1LG2 may have immune function, IFNL3 encodes an immune cytokine, ACTBL2 may be important for cellular movement especially for muscle cells, ANKRD34B expression levels may vary with tissue and/or cancer type, KMO encodes a protein used in a metabolic pathway, HTR1D may be important for neural function, locomotion, and anxiety, CCBE1 may be important for developing extracellular matrices and expression levels may vary with tissue and/or cancer type, NETO1 may be important for neural function, spatial learning, and memory formation, KLC3 may be important for intracellular transport along microtubules, RGS20 may be important for general signal transduction from the exterior to the interior of a cell, PRSS36 expression levels may vary with tissue and/or cancer type, GTSF1 expression levels may vary with tissue and/or cancer type, SPRR1B may form the membrane of certain cells and expression levels may vary with tissue and/or cancer type, CYP27B1 may be important for drug metabolism and lipid synthesis, SDK1 may be important for cellular adhesion and immune function, GNGT1 may be important for visual perception, COPZ2 may be important for intracellular transport in COPI vesicles, PSMB8 may be important for generating peptides for presentation to immune cells especially by HLA proteins, CR2 allows viral entry into B and T cells and expression levels may vary with tissue and/or cancer type, HLA-DQA1 may be important for presenting peptides to immune cells, HMGA1 may be important for gene transcription, viral integration into human chromosomes, and cancer metastasis, ST6GALNAC5 may be important for cell to cell interactions, TCHH may be important for hair follicles and tongue papillae, HLA-DQB1 may be important for presenting peptides to immune cells, MYBPC2 may be important for muscle and heart cells, ULBP2 may be important for immune function, SCGB3A2 may be important for lung function, TMPRSS4 fragments other proteins and expression levels may vary with tissue and/or cancer type, LIPG may be important for lipoprotein metabolism and the circulatory system, CARD17 may be important for immune system function, HLA-DRB1 may be important for presenting peptides to immune cells, and AHNAK2 may be important for calcium signaling. The expression levels of many of these genes may vary depending on the cell type and/or cancer type of the cell in which the gene is expressed.

In embodiment three, the following genes selected as features and/or proteins encoded by these genes have these general functions, according to published research: CD274 encodes immune inhibitor PD-L1, CEACAM21 may regulate the immune system and cell adhesion and is expressed by fetal tissue but certain cancer types may increase expression levels in adults, LRRC37A2 expression levels may vary with tissue and/or cancer type, RNASE10 may be important for sperm maturation, PSG4 may regulate the innate immune system and cell adhesion and is expressed by fetal tissue but certain cancer types may increase expression levels in adults, SYT1 may be important for neural function, intracellular trafficking and exocytosis, HTR1D may be important for neural function, locomotion, and anxiety, IL31RA encodes an immune cytokine receptor, TRIM50 may be important for the modification and degradation of other proteins, ANKRD2 may be important for muscle function, SLC25A41 may be important for transporting molecules into and out of cell mitochondria, GAGE12H is expressed by germ cells including ova and sperm but certain cancer types may increase expression levels in other cell types, CPA5 may be important for digesting food and synthesizing peptides, GAGE12C is expressed by germ cells including ova and sperm but certain cancer types may increase expression levels in other cell types, GAGE12D is expressed by germ cells including ova and sperm but certain cancer types may increase expression levels in other cell types, FKBPL may be important for immune function and cell cycle regulation, PSG5 is expressed by placental tissue but certain cancer types may increase expression levels in other tissue types, NCF1 encodes an oxidase for neutrophils and has some immune functions, IL21 encodes an immune cytokine, CYP2A13 may be important for drug metabolism and lipid synthesis, NOXO1 may be important for a type of metabolism known as respiratory burst, ANKRD34B expression levels may vary with tissue and/or cancer type, ITPKA may be important for metabolizing inositol phosphate for cell signaling, HLA-DPB1 may be important for presenting peptides to immune cells, PSG9 may regulate blood platelet adhesion and is expressed by placental tissue but certain cancer types may increase expression levels in other tissue types, CAMK1G may be involved in intracellular signaling, HMGN5 may bind to the nucleosome and activate gene transcription, BTG4 regulates the cell cycle and cell division, FGF5 may be important for fetal development, cell growth, and tumor growth, KRT24 may be important for epithelial cell structure, SAXO2 may be important for microtubules and intracellular transport, CLLU1OS expression levels may vary with tissue and/or cancer type, KRT31 may be important for hair and nail growth, PAGE1 expression levels may vary with tissue and/or cancer type, PRM3 may be important for DNA packaging in sperm and expression levels may vary with tissue and/or cancer type, LRRC37A expression levels may vary with tissue and/or cancer type, ANKRD18B may bind to nucleotides, PSG2 may regulate cell adhesion and is expressed by placental tissue but certain cancer types may increase expression levels in other tissue types, AFAP1L2 may be important for cell signaling pathways, and DMRTA2 may be important for DNA binding and transcription. The expression levels of many of these genes may vary depending on the cell type and/or cancer type of the cell in which the gene is expressed.

The labeled data set 1301 may be reduced to a filtered data set 1410 having data associated with the selected features for each cancer cell sample and the PD-L1 status predictor 115 may be trained only on the selected features in the filtered data set 1410.

### Analyzing Model Accuracy for Example Embodiments of PD-L1 Status Predictor 1115

In one example, only a portion of the cancer cell samples in each fold are used to train the PD-L1 status predictor, and the other cancer cell samples in the fold are called withheld cancer cell samples in a validation sample. The withheld cancer cell samples in the validation sample are used as input cases 1112 to test the PD-L1 status predictor 1115, which does not receive the PD-L1 status 1305 associated with the withheld cancer cell samples. The PD-L1 status predictor 1115 generates a predicted PD-L1 status 1120 for each withheld cancer cell sample and it is compared to the actual PD-L1 status 1305 associated with the withheld cancer cell sample to determine the accuracy of the trained predictive model 1420.

A labeled data set 1301 may be divided into any number of folds for feature selection. In one example, the number of folds may be chosen to increase the likelihood that the validation sample has at least one cancer cell sample associated with a positive PD-L1 status 1305. In one example, there are five folds.

The ratio of the number of cancer cell samples in the validation sample over the number of cancer cell samples in the fold may be equal to the ratio of the number of cancer cell samples in the fold over the number of cancer cell samples in the training data set. For example, if there are five folds, the validation sample of each fold may contain ⅕ of the cancer cell samples in the fold.

In one example, model accuracy analysis 1422 includes plotting a Receiver Operating Characteristic (ROC) curve and calculating the area under curve (AUC) to analyze the performance of a trained predictive model on the validation sample. The AUC indicates the probability that the model will rank a randomly selected cancer cell sample associated with a negative PD-L1 status 1305 as more PD-L1 negative than a randomly selected cancer cell sample associated with a positive PD-L1 status 1305. The maximum possible value for an AUC is 1. An AUC of 1 indicates a perfectly accurate model, and the higher the AUC, the more useful the model is. An ROC curve may be generated for the validation sample of each fold. For imbalanced data, it is also useful to evaluate model performance using a precision-recall curve. This curve also has a maximum value of 1, where higher values indicate better model performance.

In one example, the ROC curve for embodiment one (CD274 model) has an AUC of 0.84 for fold 1, 0.93 for fold 2, 0.89 for fold 3, 0.90 for fold 4, 0.96 for fold 5, and a mean of 0.90+/−0.04 for all five folds. Embodiment one has an accuracy of 0.914, precision of 0.817, recall of 0.525, and adjusted mutual information of 0.323.

In one example, the ROC curve for embodiment two (median difference model) has an AUC of 0.92 for fold 1, 0.99 for fold 2, 0.92 for fold 3, 0.98 for fold 4, 0.88 for fold 5, and a mean of 0.94+/−0.04 for all five folds. Embodiment two has an accuracy of 0.919, precision of 0.750, recall of 0.622, and adjusted mutual information of 0.357.

In one example, the ROC curve for embodiment three (variance model) has an AUC of 0.93 for fold 1, 0.94 for fold 2, 0.93 for fold 3, 0.98 for fold 4, 0.94 for fold 5, and a mean of 0.94+/−0.02 for all five folds. Embodiment three has an accuracy of 0.911, precision of 0.697, recall of 0.632, and adjusted mutual information of 0.315.

### Patient Example

In one example of the application, features are selected from a published list of genes that were observed by a third party to be correlated with PD-L1 expression levels or PD-L1 status. A logistic regression model 1415 is trained on a set of training data containing 595 cases of labeled PD-L1 positive and PD-L1 negative samples associated with RNA-seq data, using the gene expression values of the following genes: CD274, PDCD1, PDCD1 LG2, IFNG, GZMB, CXCL9, TGFB1, VIM, STX2, and ZEB2.

In this embodiment all selected features are expression level values of genes, including but not limited to the following genes: CD274 (1.072), PDCD1(−0.168), PDCD1 LG2 (0.357), IFNG (−0.076), GZMB (−0.121), CXCL9 (0.200), TGFB1 (−0.063), VIM (−0.248), STX2 (0.316), and ZEB2 (−0.090). The value denoted in parentheses after a gene name indicates the approximate feature weight value for that gene expression level in the logistic regression function. In this example, the selected features are expression levels of genes that were published in scientific literature as being correlated with PD-L1 status and/or PD-L1 protein expression levels.

In one example case, consider a patient with metastatic melanoma. A sample of the patient's tumor is then sequenced, yielding DNA and RNA sequencing data.

Normalized gene counts for all genes are obtained from the RNA-sequencing data and downstream pipeline. A subset of these genes (including features previously determined to be the most informative for predicting PD-L1 IHC status) are used as input into the prediction model.

In this example, the normalized gene counts of the genes used to predict PD-L1 status are: CD274: 2.11, PDCD1: 2.15, PDCD1LG2: 2.15, IFNG, 2.12, GZMB, 2.76, CXCL9: 3.51, TGFB1: 3.02, VIM: 4.28, STX2: 2.37, and ZEB2: 3.43.

To create a data set that will be received by PD-L1 predictor 1115 as input case 1112, these gene counts are then mean centered and rescaled as described above (See FIG. 6A) by Scikit-learn tools to match the data distribution in the training set in terms of mean and unit variance. In this example, input case 1112 contains the following standardized, rescaled counts of the genes used to predict PD-L1 status are: CD274: 2.88, PDCD1: 1.90, PDCD1LG2: 2.01, IFNG: 2.29, GZMB: 1.84, CXCL9: 2.23, TGFB1: 0.57, VIM: 0.41, STX2: 1.38, and ZEB2: 0.29.

From the gene expression levels of CD274 and the other genes in this list that provide information on PD-L1 status, the PD-L1 predictor 1115 calculates the PD-L1 status prediction probability 1122 of the patient's cancer by multiplying each standardized, rescaled count by the corresponding feature weight and calculating the sum of these products and the intercept value from the logistic regression function. Then the PD-L1 predictor 1115 classifies the predicted probability 1122 as a Negative, Equivocal, or Positive predicted PD-L1 status label 1120 as described above (See FIG. 6C).

In this example, the PD-L1 positive prediction probability 1122 returned by the model is: 0.97, thus the patient would be classified as PD-L1 positive with high confidence. Here, high confidence may mean a positive result prediction probability 1122 of greater than 0.8—this prediction probability threshold was obtained by the error evaluation method described above. (See FIG. 2C)

The patient's medical record 1325 could then be searched for relevant information, such as a prior treatment with pembrolizumab or another immune checkpoint blockade therapy. If prior treatment with pembrolizumab is found, one example of report logic will ensure that pembrolizumab is not displayed as a therapy option in the report 1125. Instead, a combination therapy of atezolizumab and ipilimumab could be displayed, for example, depending on additional information derived from the patient's molecular data that support the use of an alternate checkpoint blockade strategy.

The appearance of predicted PD-L1 status 1120 in the report 125 may include the patient's PD-L1 RNA expression and a visualization of other patients of a similar cancer type or a reference set of samples from a normal tissue type selected to be a reference for the particular cancer type. (See FIG. 5B)

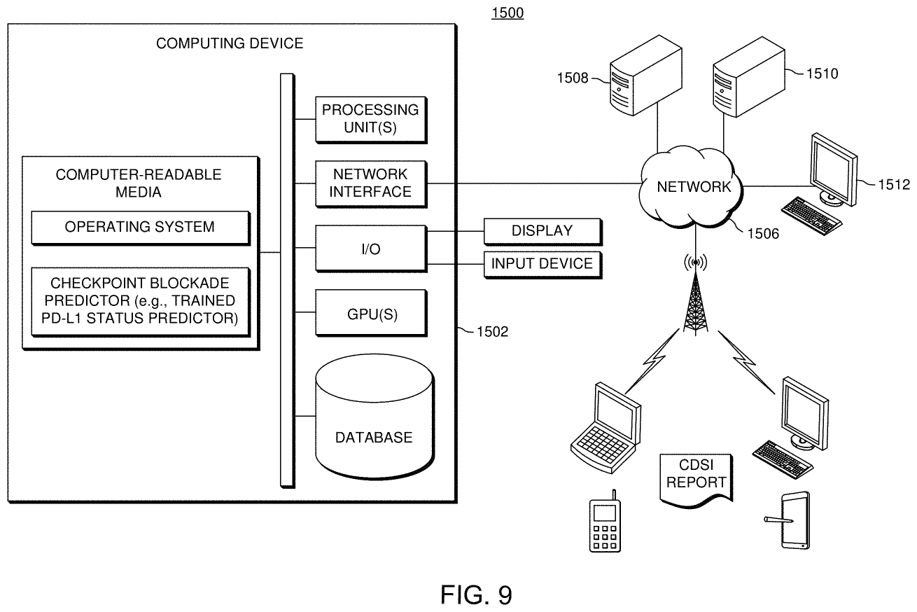

The techniques herein, including the PD-L1 status predictor techniques, may be implemented on any number of computing systems described herein, including, for example, the system 100 of FIG. 1. In the illustrated example of FIG. 9, the techniques herein are implemented on genetic sequence analysis processing system 1500 that may be implemented on a computing device 1502 such as a computer, tablet or other mobile computing device, or server, such as a cloud-based server. The genetic sequence analysis processing system 1500 may include a number of processors, controllers or other electronic components for processing sequence data and performing the processes described herein. The genetic sequence analysis processing system 1500, for example, may be implemented on a one or more processing units, which may represent Central Processing Units (CPUs), and/or on one or more or Graphical Processing Units (GPUs), including clusters of CPUs and/or GPUs. Features and functions described for the genetic sequence analysis processing system 1500 may be stored on and implemented from one or more non-transitory computer-readable media of the computing device 1502. The computer-readable media may include, for example, an operating system and elements corresponding to the processes described herein, and in reference to FIGS. 5A-8. For example, the computer-readable media may store (and in some examples generate) trained PD-L1 status predictor models, executable code, etc. use for implementing the techniques herein, including genetic sequence data analysis. The computer-readable media may store any suitable checkpoint blockade predictor, in accordance with the examples herein. The computing device 1502 may include a network interface communicatively coupled to a network 1506, for communicating to and/or from a portable personal computer, smart phone, electronic document, tablet, and/or desktop personal computer, or other computing devices. The computing device 1502 may further include an I/O interface connected to devices, such as digital displays, user input devices, etc.

In some examples, the genetic sequence analysis processing system 1500 is implemented on a single server, such as a single cloud-based server. However, the functions of the system may be implemented across distributed devices such as network-accessible servers 1508, 1510, and 1512, etc. connected to one another through a communication link or cloud-based infrastructure. In other examples, functionality of the genetic sequence analysis processing system 1500 may be distributed across any number of devices, including the portable personal computer, smart phone, electronic document, tablet, and desktop personal computer devices shown.

The network 1506 may be a public network such as the Internet, private network such as research institutions or corporations private network, or any combination thereof. Networks can include, local area network (LAN), wide area network (WAN), cellular, satellite, or other network infrastructure, whether wireless or wired. The network 1506 can utilize communications protocols, including packet-based and/or datagram-based protocols such as internet protocol (IP), transmission control protocol (TCP), user datagram protocol (UDP), or other types of protocols. Moreover, the network 1506 can include a number of devices that facilitate network communications and/or form a hardware basis for the networks, such as switches, routers, gateways, access points (such as a wireless access point as shown), firewalls, base stations, repeaters, backbone devices, etc. In some example “cloud-based” implementations, functionality of the genetic sequence analysis processing system, including that PD-L1 status prediction, may be implemented as part of a “cloud” network, with hardware devices and/or software components within the cloud network to send, retriever, analyze, generate, and report data. In some examples, a cloud-based implementation of the genetic sequence analysis processing system 1500 may operate as a Software-as-a-Service (SaaS) or Platform-as-a-Service (PaaS), providing the functionality described herein remotely to software apps and other components in accordance with the various embodiments described herein.

In some examples, the genetic sequence analysis processing system 1500 implements a method of preparing a clinical decision support information (CDSI) report as shown. For example, a subject's tissue sample may be received at the genetic sequence analysis processing system 1500, which then determines or identifies a PD-L1 expression status of the subject's sample, for example, by performing an alignment of an unlabeled gene expression data set of the subject's sample to a labeled expression data according to a trained PD-L1 predictive model. From that alignment comparison, the genetic sequence analysis processing system 1500 prepares an electronic CDSI report for the subject based on the PD-L1 expression status identified. That CDSI report may be communicated to network accessible devices, as shown. In some examples, the CDSI report may contain the subject's name, the PD-L1 expression status, and one or more of the date on which the sample was obtained from the subject, the sample type, a list of candidate drugs correlating with the PD-L1 expression status, data from images of the subject's tumor or cancer, image features, clinical data of the subject, epigenetic data of the subject, data from the subject's medical history and/or family history, subject's pharmacogenetic data, subject's metabolomics data, etc.

Further, in some examples, the genetic sequence analysis processing system 1500 prepares an initial digital (preliminary) CDSI report for the subject based on the PD-L1 expression status identified. The genetic sequence analysis processing system 1500 compares the initial CDSI report against stored clinical data for the patient. The genetic sequence analysis processing system 1500 determines from the comparison if a further modification or optimization should be performed on the CDSI report before it is communicated to the subject or medical professionals, for example, over the network 1506. In some examples, the initial CDSI report may include a list of candidate drugs correlating with PD-L1 expression status. The genetic sequence analysis processing system 1500 compares the list of candidate drugs to a listing of drugs previously administered to the subject and determines if the list of candidate drugs should be changed, e.g., reduced to remove drugs already administered to the subject.

In some aspects, the systems and methods disclosed herein may be used to support clinical decisions for personalized treatment of cancer. For example, in some embodiments, the methods described herein identify actionable genomic variants and/or genomic states with associated recommended cancer therapies. In some embodiments, the recommended treatment is dependent upon whether or not the subject has a particular actionable variant and/or genomic status. Recommended treatment modalities can be therapeutic drugs and/or assignment to one or more clinical trials. Generally, current treatment guidelines for various cancers are maintained by various organizations, including the National Cancer Institute and Merck & Co., in the Merck Manual.

In some embodiments, the methods described herein further includes assigning therapy and/or administering therapy to the subject based on the identification of an actionable genomic variant and/or genomic state, e.g., based on whether or not the subject's cancer will be responsive to a particular personalized cancer therapy regimen. For example, in some embodiments, when the subject's cancer is classified as having a first actionable variant and/or genomic state, the subject is assigned or administered a first personalized cancer therapy that is associated with the first actionable variant and/or genomic state, and when the subject's cancer is classified as having a second actionable variant and/or genomic state, the subject is assigned or administered a second personalized cancer therapy that is associated with the second actionable variant. Assignment or administration of a therapy or a clinical trial to a subject is thus tailored for treatment of the actionable variants and/or genomic states of the cancer patient.

As discussed herein, the computer-readable media may include executable computer-readable code stored thereon for programming a computer (e.g., comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device may represent a CPU-type processing unit, a GPU-type processing unit, a field-programmable gate array (FPGA), another class of digital signal processor (DSP), or other hardware logic components that can be driven by a CPU.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components or multiple components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a microcontroller, field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a processor configured using software, the processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein can be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method can be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines, including distributed across a “cloud” network. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternate embodiments, using either current technology or technology developed after the filing date of this application.

All references, including publications, patent applications, and patents, cited herein are hereby incorporated by reference to the same extent as if each reference were individually and specifically indicated to be incorporated by reference and were set forth in its entirety herein.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A computer-implemented method of identifying programmed-death ligand 1 (PD-L1) expression status of a subject's sample comprising a cancer cell, the method comprising:
receiving an unlabeled expression data set for the subject's sample; and
aligning the unlabeled expression data set to labeled expression data according to a trained PD-L1 predictive model, wherein the trained PD-L1 predictive model has been trained with a plurality of labeled expression data sets, each labeled expression data set comprising expression data for a sample of a labeled PD-L1 expression status;
wherein aligning the unlabeled gene expression data set to labeled expression data according to the trained PD-L1 predictive model identifies PD-L1 expression status for the subject's sample.

2. The computer-implemented method of claim 1, wherein the trained PD-L1 predictive model has been trained by (i) inputting a plurality of labeled expression data sets, wherein each labeled expression data set comprises a labeled cancer type and a labeled PD-L1 expression status, and, optionally, one or more labeled features.

3. The computer-implemented method of claim 2, wherein the plurality of labeled expression data sets comprises expression data for samples of a single cancer type.

4. The computer-implemented method of claim 2, wherein the plurality of labeled expression data sets comprises expression data for samples of 2, 3, 4, 5, 6, 7, 8, 9, 10 or more different cancer types.

5. (canceled)

6. (canceled)

7. The computer-implemented method of claim 2, wherein the plurality of labeled expression data sets comprises expression data for breast cancer, prostate cancer, colorectal cancer, lung cancer, skin cancer, kidney cancer, pancreatic cancer, stomach cancer, or a combination thereof.

8. The computer-implemented method of claim 2, wherein the plurality of labeled expression data sets comprises expression data for a subtype of one or more of labeled cancer type(s), optionally, a subtype of breast cancer.

9. The computer-implemented method of claim 8, wherein the subtype for breast cancer is luminal breast cancer, triple negative breast, or a combination thereof.

10. The computer-implemented method of claim 2, wherein the plurality of labeled expression data sets comprises expression data for lung adenocarcinoma, melanoma, renal cell carcinoma, bladder cancer, mesothelioma, and lung small cell cancer.

11. The computer-implemented method of claim 2, wherein the labeled expression data comprises RNA expression data, optionally, mRNA expression data.

12. The computer-implemented method of claim 11, wherein the mRNA expression data is RNA-seq data.

13. The computer-implemented method of claim 12, wherein the RNA-seq data is normalized RNA-seq data.

14. The computer-implemented method of claim 1, wherein the unlabeled expression data set for the sample comprises RNA expression data, optionally, mRNA expression data.

15. The computer-implemented method of claim 14, wherein the mRNA expression data is RNA-seq data.

16. The computer-implemented method of claim 15, wherein the RNA seq data is normalized RNA-seq data.

17. The computer-implemented method of claim 1, further comprising one or more of: obtaining the sample from a subject, isolating mRNA from cells of the sample, fragmenting the mRNA, producing double-stranded cDNA based on the mRNA fragments, carrying out high throughput, short-read sequencing on the cDNA, aligning the sequences to a reference genome, and normalizing raw RNA-seq data.

18. The computer-implemented method of claim 17, wherein the high throughput, short-read sequencing is next generation sequencing (NGS), optionally, wherein the NGS comprises hybrid capture.

19. The computer-implemented method of claim 18, wherein the hybrid capture comprises use of biotinylated probes which bind to specific target nucleotide sequences.

20. The computer-implemented method of claim 19, wherein at least one of the target nucleotide sequences encodes PD-L1, PD-1, or a combination thereof.

21. The computer-implemented method of claim 19, wherein at least one of the target nucleotide sequences encodes 4-1BB, TIM-3, or other immune checkpoint blockade molecules.

22. The computer-implemented method of claim 2, wherein each labeled expression data set further comprises data from images, image features, clinical data, epigenetic data, pharmacogenetic data, metabolomics data, or a combination thereof.

23. The computer-implemented method of claim 1, wherein the unlabeled expression data set further comprises data from images, image features, clinical data, epigenetic data, pharmacogenetic data, metabolomics data, or a combination thereof, of the subject.

24. The computer-implemented method of claim 1, wherein the trained PD-L1 predictive model has been trained with a plurality of labeled expression data sets, each labeled expression data set comprises one or more labeled features, and the trained PD-L1 predictive model has been trained according to select labeled features pre-determined to have an association with a phenotype of biological relevance.

25. The computer-implemented method of claim 24, wherein the trained PD-L1 predictive model was trained using a clustering algorithm to determine which labeled features associate with the phenotype of biological relevance.

26. The computer-implemented method of claim 25, wherein the phenotype of biological relevance is PD-L1 expression status.

27. The computer-implemented method of claim 24, wherein the at least one or more of the select labeled features comprises expression data for at least one gene selected from the group consisting of CD274, TIGIT, CXCL13, IL21, FASLG, TFPI2, GAGE12C, POMC, PAX6, NPHS1, HLA-DPB1, PDCD1, PDCD1LG2, IFNG, GZMB, CXCL9, TGFB1, VIM, STX2, and ZEB2.

28. The computer-implemented method of claim 1, wherein the trained PD-L1 predictive model is a logistic regression model, a random forest model, or a support vector machine (SVM) model, optionally, wherein the logistic regression model is a single-gene or multi-gene logistic regression model.

29. The computer-implemented method of claim 1, wherein the labeled PD-L1 expression status is based on an reverse phase protein array (RPPA) data, fluorescence in situ hybridization (FISH) data, immunohistochemistry (IHC) data, or a combination thereof, optionally, wherein the trained PD-L1 predictive model correlates the labeled PD-L1 expression status with select labeled expression data and/or labeled features.

30. The computer-implemented method of claim 1, the method further comprising:
generating a clinical decision support information (CDSI) report including at least the subject's identity and the identified PD-L1 expression status, and, optionally, providing the CDSI report to a healthcare provider for use in selecting a candidate therapy based on the identified PD-L1 expression status for the subject's sample.

31. (canceled)

32. (canceled)

33. (canceled)

34. (canceled)

35. (canceled)

36. (canceled)

37. (canceled)

38. (canceled)

39. (canceled)

40. (canceled)
